SEQUENCE LISTING (23 sequences)

SEQ ID NO: 1 - DNA, 20 bp, Artificial Sequence
  P5 primer
aatgatacggcgaccaccga

SEQ ID NO: 2 - DNA, 24 bp, Artificial Sequence
  P7 primer
caagcagaagacggcatacgagat

SEQ ID NO: 3 - protein, 19 aa, Artificial Sequence
  FS-peptide
FSGVAMPGAEDDVVGSGSK

SEQ ID NO: 4 - protein, 20 aa, Artificial Sequence
  AFS-peptide
AFSGVAMPGAEDDVVGSGSK

SEQ ID NO: 5 - protein, 21 aa, Artificial Sequence
  AEFS-peptide
AEFSGVAMPGAEDDVVGSGSK

SEQ ID NO: 6 - protein, 22 aa, Artificial Sequence
  Assay peptide
  MISC_FEATURE (22)..(22): C-terminal lysine with an azide substitution on the side chain
YAEALAESAFSGVARGDVRGGK

SEQ ID NO: 7 - protein, 21 aa, Artificial Sequence
  Assay peptide
  MISC_FEATURE (21)..(21): C-terminal lysine with an azide substitution on the side chain
AEALAESAFSGVARGDVRGGK

SEQ ID NO: 8 - protein, 20 aa, Artificial Sequence
  Assay peptide
  MISC_FEATURE (20)..(20): C-terminal lysine with an azide substitution on the side chain
EALAESAFSGVARGDVRGGK

SEQ ID NO: 9 - protein, 19 aa, Artificial Sequence
  Assay peptide
  MISC_FEATURE (19)..(19): C-terminal lysine with an azide substitution on the side chain
ALAESAFSGVARGDVRGGK

SEQ ID NO: 10 - protein, 18 aa, Artificial Sequence
  Assay peptide
  MISC_FEATURE (18)..(18): C-terminal lysine with an azide substitution on the side chain
LAESAFSGVARGDVRGGK

SEQ ID NO: 11 - protein, 17 aa, Artificial Sequence
  Assay peptide
  MISC_FEATURE (17)..(17): C-terminal lysine with an azide substitution on the side chain
AESAFSGVARGDVRGGK

SEQ ID NO: 12 - protein, 16 aa, Artificial Sequence
  Assay peptide
  MISC_FEATURE (16)..(16): C-terminal lysine with an azide substitution on the side chain
ESAFSGVARGDVRGGK

SEQ ID NO: 13 - protein, 15 aa, Artificial Sequence
  Assay peptide
  MISC_FEATURE (15)..(15): C-terminal lysine with an azide substitution on the side chain
SAFSGVARGDVRGGK

SEQ ID NO: 14 - protein, 14 aa, Artificial Sequence
  Assay peptide
  MISC_FEATURE (14)..(14): C-terminal lysine with an azide substitution on the side chain
AFSGVARGDVRGGK

SEQ ID NO: 15 - protein, 13 aa, Artificial Sequence
  Assay petide
  MISC_FEATURE (13)..(13): C-terminal lysine with an azide substitution on the side chain
FSGVARGDVRGGK

SEQ ID NO: 16 - protein, 12 aa, Artificial Sequence
  Assay peptide
  MISC_FEATURE (12)..(12): C-terminal lysine with an azide substitution on the side chain
SGVARGDVRGGK

SEQ ID NO: 17 - protein, 21 aa, Artificial Sequence
  Assay peptide
  MISC_FEATURE (21)..(21): C-terminal lysine with an azide substitution on the side chain
LAGELAGELAGEIRGDVRGGK

SEQ ID NO: 18 - protein, 22 aa, Artificial Sequence
  Assay peptide
  MISC_FEATURE (22)..(22): C-terminal lysine with an azide substitution on the side chain
ELAGELAGELAGEIRGDVRGGK

SEQ ID NO: 19 - protein, 23 aa, Artificial Sequence
  Assay peptide
  MISC_FEATURE (23)..(23): C-terminal lysine with an azide substitution on the side chain
GELAGELAGELAGEIRGDVRGGK

SEQ ID NO: 20 - protein, 24 aa, Artificial Sequence
  Assay peptide
  MISC_FEATURE (24)..(24): C-terminal lysine with an azide substitution on the side chain
AGELAGELAGELAGEIRGDVRGGK

SEQ ID NO: 21 - protein, 19 aa, Artificial Sequence
  Assay peptide
  MISC_FEATURE (19)..(19): C-terminal lysine with an azide substitution on the side chain
FAFAGVAMPRGAEDVRGGK

SEQ ID NO: 22 - protein, 13 aa, Artificial Sequence
  Assay peptide
  MISC_FEATURE (13)..(13): C-terminal lysine with an azide substitution on the side chain
FLAEIRGDVRGGK

SEQ ID NO: 23 - protein, 28 aa, Artificial Sequence
  Assay peptide
  MISC_FEATURE (1)..(1): N-terminal dimethyl group
  MISC_FEATURE (28)..(28): C-terminal lysine with an azide substitution on the side chain
AESAESASRFSGVAMPGAEDDVVGSGSK

(19) United States
(12) Patent Application Publication
(10) Pub. No.: US 2024/0042446 A1
(43) Pub. Date: Feb. 8, 2024

(54) AUTOMATED TREATMENT OF MACROMOLECULES FOR ANALYSIS AND RELATED APPARATUS

(71) Applicant: Encodia, Inc., San Diego, CA (US)

(72) Inventors: Timothy Scott BURCHAM, San Diego, CA (US); Mark S. CHEE, San Diego, CA (US); Kevin L. GUNDERSON, San Diego, CA (US)

(73) Assignee: Encodia, Inc., San Diego, CA (US)

(21) Appl. No.: 17/769,321

(22) PCT Filed: Oct. 14, 2020
(86) PCT No.: PCT/US2020/055612

§ 371 Date: Apr. 14, 2022

Related U.S. Application Data
(60) Provisional application No. 62/923,406, filed on Oct. 18, 2019.

(51) Int. Cl. B01L 3/00; G01N 35/10

(52) U.S. Cl. CPC B01L 3/50851; G01N 35/1002; G01N 2035/00405

(57) ABSTRACT

The present disclosure relates to an apparatus for preparing and treating macromolecules, e.g., peptides, polypeptides, and proteins for sequencing and/or analysis. An automated method for performing an automated assay for macromolecule analysis includes, inter alia, moving each of a plurality of reagents to a sample containing a solid support material and incubating the various reagents with the sample is provided. In some embodiments, the apparatus and automated methods are for use in treating and modifying a macromolecule or a plurality of macromolecules, (e.g., peptides, polypeptides, and proteins) for sequencing and/or analysis that employ barcoding and nucleic acid encoding of molecular recognition events, and/or detectable labels.

### RELATED APPLICATION

The present application claims priority to U.S. provisional patent application No. 62/923,406, filed on Oct. 18, 2019, the disclosures and contents of which are incorporated herein by reference in their entireties for all purposes.

### SEQUENCE LISTING ON ASCII TEXT

This patent application file contains a Sequence Listing submitted in computer readable ASCII text format (file name: 4614-2001940_SeqList_ST25.txt, date recorded: 12 Oct., 2020, size: 8,703 bytes). The content of the Sequence Listing file is incorporated herein by reference in its entirety.

### TECHNICAL FIELD

The present disclosure relates to an apparatus for preparing and/or treating macromolecules, e.g., peptides, polypeptides, and proteins for sequencing and/or other analysis. Also provided is an automated method for performing an assay for macromolecule analysis which includes moving each of a plurality of reagents to a sample containing immobilized macromolecules and incubating the various reagents with the sample. In some embodiments, the apparatus and automated methods are for use in treating and/or modifying a macromolecule or a plurality of macromolecules, (e.g., peptides, polypeptides, and proteins) for sequencing and/or other analysis which employs barcoding and nucleic acid encoding of molecular recognition events, and/or detectable labels.

### BACKGROUND

Existing technologies for analyzing macromolecules such as proteins or peptides are limited in several ways. Molecular recognition and characterization of a protein or peptide macromolecule is typically performed using an immunoassay including formats such as ELISA, multiplex ELISA (e.g., spotted antibody arrays, liquid particle ELISA arrays), digital ELISA, reverse phase protein arrays (RPPA), and others. These different immunoassay platforms share similar challenges including the development of high affinity and highly-specific or selective antibodies (binding agents), limited ability to multiplex at both the sample and analyte level, limited sensitivity and dynamic range, and cross-reactivity and background signals. Binding agent agnostic approaches such as direct protein characterization via peptide sequencing (e.g., Edman degradation or Mass Spectroscopy) provide alternative approaches. However, neither of these approaches is very parallel or high-throughput. Peptide sequencing based on Edman degradation includes stepwise degradation of the N-terminal amino acid on a peptide through a series of chemical modifications and downstream HPLC analysis (later replaced by mass spectrometry analysis). However, in general, Edman degradation peptide sequencing is slow and has a limited throughput. Other existing methodologies include electrospray mass spectroscopy (MS), and LC-MS/MS. However, MS is limited by drawbacks including high instrument cost, requirement for a sophisticated user, poor quantification ability, and limited ability to make measurements spanning the dynamic range of the proteome. For MS, sample throughput is typically limited to a few thousand peptides per run, and for data independent analysis (DIA), this throughput is inadequate for true bottoms-up high-throughput proteome analysis.

Accordingly, a need exists for an apparatus and methods for automated treatment and/or preparation of samples to achieve proteomics technology that is highly-parallelized, accurate, sensitive, and high-throughput. The present disclosure fulfills these and other related needs. For example, the provided automated instrument and methods addresses concerns associated with manual approaches to preparing and treating samples for a macromolecule analysis assay. In particular, significant advantages can be realized by automating the various process steps of a macromolecule analysis assay, including greatly reducing the risk of user-error, contamination, and spillage, increasing accuracy and control across treatment of samples, while significantly increasing through-put volume. Automating the steps of a macromolecule analysis assay will also reduce the amount of training required for practitioners and remove sources of physical injury attributable to high-volume manual applications.

These and other aspects of the invention will be apparent upon reference to the following detailed description. To this end, various references are set forth herein which describe in more detail certain background information, procedures, compounds and/or compositions, and are each hereby incorporated by reference in their entirety.

### BRIEF SUMMARY

The summary is not intended to be used to limit the scope of the claimed subject matter. Other features, details, utilities, and advantages of the claimed subject matter will be apparent from the detailed description including those aspects disclosed in the accompanying drawings and in the appended claims.

Provided herein is an apparatus for automated treatment of a sample containing an immobilized macromolecule. The apparatus includes one or more non-planar sample container(s) with a volume equal to or less than about 20 mL, wherein at least one of said sample container(s) is subjected to temperature control and configured for allowing fluid flow-through, or a holder or space configured for holding said sample container(s); a plurality of reagent reservoirs for containing a respective reagent, wherein at least one of said reagent reservoirs is subjected to temperature control, or a holder or space configured for holding said reagent reservoir(s); a plurality of valves connected in a supply line having an upstream end and a downstream end, wherein at least one or each of said valves is positionable to provide alternate flow paths therethrough; and a control unit to control delivery of said one or more reagent(s) to said sample container(s), wherein delivery of said one or more reagent is individually addressable, said supply line connects said reagent reservoirs to said sample container(s) and said reagent reservoirs are fluidically connected to said sample container(s), and at least temperature control of said sample container(s), temperature control of said reagent reservoir(s), positioning of said valve(s) and/or delivery of said one or more reagent(s) to said sample container(s) is automated and controlled by said control unit.

Provided herein is a method for automated treatment of a sample, which method is conducted using an apparatus, and which method comprises: a) providing a non-planar sample container comprising a sample comprising a macromolecule, e.g., a polypeptide, and an associated recording tag joined to a solid support to said apparatus; b) providing a binding agent and reagents for transferring information to separate reagent reservoirs of said apparatus, wherein at least one of said reagent reservoirs comprises a binding agent and at least one of said reagent reservoirs comprises reagents for transferring information; c) delivering the binding agent from the reagent reservoir to the sample container, wherein the binding agent comprises a coding tag with identifying information regarding the binding agent; and d) delivering the reagents for transferring information from the reagent reservoir to the sample container to transfer information from the coding tag of the binding agent to the recording tag to generate an extended recording tag. In some embodiments, the method further includes providing reagents for removing a terminal amino acid of a polypeptide to a separate reagent reservoir of said apparatus and delivering the reagents for removing a terminal amino acid of a polypeptide from the reagent reservoir to the sample container to remove the terminal amino acid of a polypeptide. In some embodiments, the method further includes providing reagents for a capping reaction to a separate reagent reservoir of said apparatus and delivering the reagents for a capping reaction from the reagent reservoir to the sample container.

### BRIEF DESCRIPTION OF THE DRAWINGS

Non-limiting embodiments of the present invention will be described by way of example with reference to the accompanying figures, which are schematic and are not intended to be drawn to scale. For purposes of illustration, not every component is labeled in every figure, nor is every component of each embodiment of the invention shown where illustration is not necessary to allow those of ordinary skill in the art to understand the invention.

FIG. 1A-1C illustrate an exemplary system 100 for preparing macromolecules (e.g. polypeptides). The system includes n number of reagent reservoirs 101 each connected to controlled valves 102 which may be opened or closed for the delivery of various reagents from each of the reservoirs. The reagent reservoirs and valves are fluidically connected to a pump 103 which is connected to n number of sample containers, e.g. cartridges, 105. The sample containers are contained in a temperature controlled unit 104. The reagents may include wash buffers, polypeptides, nucleic acids, binding agents, enzymes, chemical or enzymatic reagents for cleaving a terminal amino acid, and/or reagents for a ligation or polymerase-mediated reaction. As shown in FIG. 1A-1C, a series of fluidic connections 107 connects each of the reagents 101 to the pump 103, to each of the sample container(s) 105, and to a waste container 106. In some embodiments, the sample container is or comprises a cartridge comprising a filter means or a frit for retaining the sample while allowing flow-through of other materials (e.g. buffers). In some cases, the sample comprises macromolecules (e.g. polypeptides) joined to a solid support. A control system 108 controls various components of the system 100 including for example, the valve(s) and pump with respect to the dispensing and flow of the reagents. In some embodiments, the control system also receives feedback from various components of the system including one or more of the valves 102, the temperature controlled unit 104, and/or the sample container(s) 105. In some embodiments, the components that are controlled or in communication with the control system are shown or illustrated with the dashed box and all the electronic components would be in connection with the control system.

In FIG. 1A, an exemplary system is depicted where all reagent valves and sample container (e.g. cartridge) valves are closed and the pump delivers bypass to the waste container. In FIG. 1B, an exemplary system is depicted where the pump aspirates one reagent. In FIG. 1C, an exemplary system is depicted where the pump delivers a reagent from the reagent reservoir to the sample container (e.g. cartridge).

FIG. 1D is a diagram of an exemplary microwave reactor for applying microwave energy to a sample container (e.g. cartridge). A solid-state MW generator is used to apply MW energy to a single mode resonant cavity. In a preferred mode, the MW Generator operates at 2.45 GHz+−0.−0.05 GHz. The dimensions of the MW cavity are designed to enable excitation of a single-mode of the cavity to create a standing wave with the electric field concentrated at the cartridge positioned in the center of the cavity. The dashed curved line in the microwave cavity indicates the time averaged absolute value of the single mode electric field intensity within the MW cavity. The intensity of the E field is maximal at the center of the cavity where the sample cartridge is positioned.

FIG. 2A is a flow diagram illustrating an exemplary process 200 for preparing macromolecules using the exemplary system 100. The method begins at 201 where one or more samples and one or more reagents (e.g. in the reagent reservoirs 101) are placed in the apparatus of the exemplary system 100. In some embodiments, the sample is loaded into a sample container (e.g. cartridge) and the cartridge is then placed in the instrument. In some embodiments, the sample comprises polypeptides prepared prior to 201, e.g. joining macromolecules in the sample to a solid support, joining macromolecules to a nucleic acid (e.g. a recording tag), digested or fragmented polypeptides, and/or treating the sample with an enzyme or a chemical agent. Once the sample is provided in the sample container, e.g. in a cartridge, the process 200 moves to prime or flush the system and fluidic connections in 202, by filling the lines with a buffer for example. The system then proceeds to 203 to set the temperature of the temperature controlled unit 104 containing the cartridge(s) and deliver a wash solution to the sample in state 204. A loop is performed comprising processes 205-207 repeated n number of times followed by a process 208. During any steps prior to 209 which requires removal of reagents or a wash, the sample container can be evacuated such that solution is removed while the sample containing the macromolecules (e.g. joined to a solid support) is retained in the sample container. The sample is removed from the sample container using any appropriate means at 209. In some embodiments, prior to or after removal of the sample from the instrument, the sample is prepared for sequencing and analysis. The process 200 may further include data analysis (e.g. using next-generation sequencing methods). The process 200 may further include delivery of other reagents, for example, delivering a reagent for modifying a terminal amino acid of a polypeptide and/or reagents for a capping reaction to the sample container.

In some embodiments, processes 205-207 or portions thereof may be modified by adding, removing, and/or switching the order of some of the steps. For example, a binding agent used in the process 205 may be configured to bind to a chemically modified amino acid treated as described in the process 207. In some workflows, one or more steps of process 207 (e.g. functionalization or modification of a terminal amino acid) may be performed prior to performing process 205 and/or 206.

FIG. 2B is a flow diagram illustrating an exemplary process 205 for delivering one or more binding agents to the sample within the process 200 for preparing macromolecules using the exemplary system 100. The process 205 includes setting the temperature of the temperature controlled unit 104 containing the cartridge(s) in state 205A and delivering a mixture containing one or more binding agents to one or more sample container(s) in state 205B and incubating the sample(s) with said mixture containing the binding agent(s). This is followed by two wash steps performed in states 205C and 205D. In some embodiments, the wash removes excess binding agents or non-specific binding. In some cases, the wash prepares the recording tag for information transfer e.g., by ligation or extension.

FIG. 2C is a flow diagram illustrating an exemplary process 206 for transferring information to recording tags within the process 200 for preparing macromolecules using the exemplary system 100. The process 206 includes setting the temperature of the temperature controlled unit 104 containing the cartridge(s) in state 206A and delivering a mixture containing reagents for transferring information (e.g. via a ligation or polymerase-mediated reaction) to the recording tags joined to the polypeptides of the sample (e.g. enzymes, nucleotides, buffers, etc.) to the sample in 206B and incubating the sample(s) with said mixture. This is followed by two wash steps and setting the temperature in states 206C, 206D and 206E.

FIG. 2D is a flow diagram illustrating an exemplary process 207 for removing a terminal amino acid (e.g. a N-terminal amino acid) within the process 200 for preparing macromolecules (e.g., polypeptides) using the exemplary system 100. In some embodiments, the terminal amino acid is removed by contacting with a chemical or enzymatic reagent. An exemplary process 207 for chemically removing a terminal amino acid is illustrated. The process 207 includes setting the temperature of the temperature controlled unit 104 containing the cartridge(s) in state 207A which is compatible with the chemical reagent used for modifying the terminal amino acid and delivering a mixture containing the chemical reagent for modifying (e.g. functionalizing) the terminal amino acid in 207B and incubating the sample(s) with said mixture. This is followed by a wash step in state 207C. The temperature of the temperature controlled unit 104 containing the cartridge(s) is then set in state 207D which is compatible with removing the terminal amino acid and the state 207E delivers a mixture containing the chemical reagent for removing or cleaving (e.g. eliminating) the terminal amino acid and incubating the sample(s) with said mixture. This is followed by setting the temperature controlled unit 104 containing the cartridge(s) and a wash in state 207G. In some embodiments, a non-modified terminal amino acid is removed. The process 207 may be modified accordingly by adding, removing, and/or switching the order of the steps.

FIG. 2E is a flow diagram illustrating an exemplary process 208 for providing a universal priming site to the recording tag within the process 200 for preparing macromolecules using the exemplary system 100. The process 208 includes setting the temperature of the temperature controlled unit 104 containing the cartridge(s) in state 208A and delivering a mixture containing reagents for providing a universal priming site to the recording tag in 208B and incubating the sample(s) with said mixture. This is followed by two wash steps and setting the temperature in states 208C, 208D and 208E. The wash steps may be useful for removing excess reagents.

Other preparation reactions and conditions, including modification, addition, or removal of steps in the method or process, are also contemplated to be within the scope of the invention. Those skilled in the art will recognize that different reagents, reaction solutions, reaction times, reaction temperatures, or sequences of reactions can be adapted for use in the invention, for example, by providing an appropriate spatial and temporal relationship between placement of components or delivery of various reagents relative to each other in accordance with the teachings herein.

FIG. 3A-3B depicts results from a polypeptide analysis assay (ProteoCode assay) performed using an exemplary apparatus to treat the tested polypeptides. The results show encoding efficiency from three cycles of binding/encoding with a binding agent (F binder) that recognizes the amino acid residue, Phenylalanine, with two cycles of treatment with a chemical reagent to remove the N-terminal amino acid (NTAA) between each binding/encoding cycle. FIG. 3A shows encoding efficiency observed in each of the three cycles with chemistry treatment between each encoding cycle and FIG. 3B shows encoding efficiency observed in each of the three cycles without any chemistry treatment for NTAA removal.

FIG. 4 depicts the demonstration of multicycle ProteoCode assay integrated on an exemplary automated fluidics apparatus using diheterocyclic methanimine (PMI) chemistry (See e.g., PCT/US2020/029969). Five cycles of ProteoCode assay are illustrated which comprised four cycles of chemistry and five cycles of binding/and encoding with a pool of two binders (F binder and L binder). The ProteoCode beads were comprised of 18 different peptides sampling F and L residues in five different positions from the N-terminus. Beads were sampled after each cycle and resultant encoded libraries analyzed with NGS sequencing. Summary NGS encoding data are shown for each of the 10 relevant F and L peptides for each cycle (only the first 5 residues shown). The F and L signal from each peptide for a given cycle corresponds to the NTAA being exposed at the particular cycle. For instance, a peptide with F in the second position (e.g., AFSGV) shows high encoding signal from the F binder in the second cycle illustrating effective peptide sequencing.

### DETAILED DESCRIPTION

Provided herein is an apparatus for preparing or treating macromolecules (e.g., peptides, polypeptides, and proteins). In some embodiments, the apparatus is used to carry out one or more steps of a macromolecule analysis assay (e.g., a polypeptide analysis assay). Also provided is a method for automated treatment of a sample comprising macromolecules. In some embodiments, one or more of the steps of the macromolecule analysis assay is automated in the provided methods, using the apparatus described herein. In some cases, the macromolecule analysis assay comprises nucleic acid encoding of molecule recognition events. In some cases, the provided apparatus is for use in treating, preparing, modifying a macromolecule from a sample for sequencing and/or analysis that employs barcoding.

Existing technologies for analyzing proteins or peptides are limited in several ways. Molecular recognition and characterization of a protein or peptide macromolecule is typically performed using an immunoassay including formats such as ELISA, multiplex ELISA (e.g., spotted antibody arrays, liquid particle ELISA arrays), digital ELISA, reverse phase protein arrays (RPPA), and others. These different immunoassay platforms share similar challenges including the development of high affinity and highly-specific (or selective) antibodies (binding agents), limited ability to multiplex at both the sample and analyte level, limited sensitivity and dynamic range, and cross-reactivity and background signals. Binding agent agnostic approaches such as direct protein characterization via peptide sequencing (e.g., Edman degradation or Mass Spectroscopy) provide alternative approaches. However, neither of these approaches is very parallel or high-throughput. Peptide sequencing based on Edman degradation includes stepwise degradation of the N-terminal amino acid on a peptide through a series of chemical modifications and downstream HPLC analysis (later replaced by mass spectrometry analysis). However, in general, Edman degradation peptide sequencing is slow and has a limited throughput. Other existing methodologies include electrospray mass spectroscopy (MS), and LC-MS/MS. However, MS is limited by drawbacks including high instrument cost, requirement for a sophisticated user, poor quantification ability, and limited ability to make measurements spanning the dynamic range of the proteome. For MS, sample throughput is typically limited to a few thousand peptides per run, and for data independent analysis (DIA), this throughput is inadequate for true bottoms-up high-throughput proteome analysis.

Accordingly, a need exists for an automated apparatus and related methods for treating and preparing samples to achieve proteomics technology that is highly-parallelized, accurate, sensitive, and high-throughput. The present disclosure fulfills these and other related needs. For example, the provided automated instrument and methods addresses concerns associated with manual approaches to preparing and treating samples for macromolecule analysis assay. In particular, significant advantages can be realized by automating the various process steps of a macromolecule analysis assay, including reducing the risk of user-error, contamination, and spillage, increasing accuracy and control across treatment of samples, while increasing through-put volume. In some cases, the automation of the assay (including settings, steps, reactions, conditions, etc.) can exhibit flexibility and allow changes to the process to be made. Automating the steps of a macromolecule analysis assay will also reduce the amount training required for practitioners and eliminate sources of physical injury attributable to high-volume manual applications.

Numerous specific details are set forth in the following description in order to provide a thorough understanding of the present disclosure. These details are provided for the purpose of example and the claimed subject matter may be practiced according to the claims without some or all of these specific details. It is to be understood that other embodiments can be used and structural changes can be made without departing from the scope of the claimed subject matter. It should be understood that the various features and functionality described in one or more of the individual embodiments are not limited in their applicability to the particular embodiment with which they are described. They instead can, be applied, alone or in some combination, to one or more of the other embodiments of the disclosure, whether or not such embodiments are described, and whether or not such features are presented as being a part of a described embodiment. For the purpose of clarity, technical material that is known in the technical fields related to the claimed subject matter has not been described in detail so that the claimed subject matter is not unnecessarily obscured.

All publications, including patent documents, scientific articles and databases, referred to in this application are incorporated by reference in their entireties for all purposes to the same extent as if each individual publication were individually incorporated by reference. Citation of the publications or documents is not intended as an admission that any of them is pertinent prior art, nor does it constitute any admission as to the contents or date of these publications or documents.

All headings are for the convenience of the reader and should not be used to limit the meaning of the text that follows the heading, unless so specified.

### Definitions

Unless defined otherwise, all technical and scientific terms used herein have the same meaning as is commonly understood by one of ordinary skill in the art to which the present disclosure belongs. If a definition set forth in this section is contrary to or otherwise inconsistent with a definition set forth in the patents, applications, published applications and other publications that are herein incorporated by reference, the definition set forth in this section prevails over the definition that is incorporated herein by reference.

As used herein, the singular forms “a,” “an” and “the” include plural referents unless the context clearly dictates otherwise. Thus, for example, reference to “a peptide” includes one or more peptides, or mixtures of peptides. Also, and unless specifically stated or obvious from context, as used herein, the term “or” is understood to be inclusive and covers both “or” and “and”.

As used herein, the term “macromolecule” encompasses large molecules composed of smaller subunits. Examples of macromolecules include, but are not limited to peptides, polypeptides, proteins, nucleic acids, carbohydrates, lipids, macrocycles. A macromolecule also includes a chimeric macromolecule composed of a combination of two or more types of macromolecules, covalently linked together (e.g., a peptide linked to a nucleic acid). A macromolecule may also include a “macromolecule assembly”, which is composed of non-covalent complexes of two or more macromolecules. A macromolecule assembly may be composed of the same type of macromolecule (e.g., protein-protein) or of two more different types of macromolecules (e.g., protein-DNA).

As used herein, the term “polypeptide” encompasses peptides and proteins, and refers to a molecule comprising a chain of two or more amino acids joined by peptide bonds. In some embodiments, a polypeptide comprises 2 to 50 amino acids, e.g., having more than 20-30 amino acids. In some embodiments, a peptide does not comprise a secondary, tertiary, or higher structure. In some embodiments, the polypeptide is a protein. In some embodiments, a protein comprises 30 or more amino acids, e.g. having more than 50 amino acids. In some embodiments, in addition to a primary structure, a protein comprises a secondary, tertiary, or higher structure. The amino acids of the polypeptides are most typically L-amino acids, but may also be D-amino acids, modified amino acids, amino acid analogs, amino acid mimetics, or any combination thereof. Polypeptides may be naturally occurring, synthetically produced, or recombinantly expressed. Polypeptides may be synthetically produced, isolated, recombinantly expressed, or be produced by a combination of methodologies as described above. Polypeptides may also comprise additional groups modifying the amino acid chain, for example, functional groups added via post-translational modification. The polymer may be linear or branched, it may comprise modified amino acids, and it may be interrupted by non-amino acids. The term also encompasses an amino acid polymer that has been modified naturally or by intervention; for example, disulfide bond formation, glycosylation, lipidation, acetylation, phosphorylation, or any other manipulation or modification, such as conjugation with a labeling component.

As used herein, the term “amino acid” refers to an organic compound comprising an amine group, a carboxylic acid group, and a side-chain specific to each amino acid, which serve as a monomeric subunit of a peptide. An amino acid includes the 20 standard, naturally occurring or canonical amino acids as well as non-standard amino acids. The standard, naturally-occurring amino acids include Alanine (A or Ala), Cysteine (C or Cys), Aspartic Acid (D or Asp), Glutamic Acid (E or Glu), Phenylalanine (F or Phe), Glycine (G or Gly), Histidine (H or His), Isoleucine (I or Ile), Lysine (K or Lys), Leucine (L or Leu), Methionine (M or Met), Asparagine (N or Asn), Proline (P or Pro), Glutamine (Q or Gln), Arginine (R or Arg), Serine (S or Ser), Threonine (T or Thr), Valine (V or Val), Tryptophan (W or Trp), and Tyrosine (Y or Tyr). An amino acid may be an L-amino acid or a D-amino acid. Non-standard amino acids may be modified amino acids, amino acid analogs, amino acid mimetics, non-standard proteinogenic amino acids, or non-proteinogenic amino acids that occur naturally or are chemically synthesized. Examples of non-standard amino acids include, but are not limited to, selenocysteine, pyrrolysine, and N-formylmethionine, 3-amino acids, Homo-amino acids, Proline and Pyruvic acid derivatives, 3-substituted alanine derivatives, glycine derivatives, ring-substituted phenylalanine and tyrosine derivatives, linear core amino acids, N-methyl amino acids.

As used herein, the term “post-translational modification” refers to modifications that occur on a peptide or protein after its translation by ribosomes is complete. A post-translational modification may be a covalent chemical modification or enzymatic modification. Examples of post-translation modifications include, but are not limited to, acylation, acetylation, alkylation (including methylation), biotinylation, butyrylation, carbamylation, carbonylation, deamidation, deiminiation, diphthamide formation, disulfide bridge formation, eliminylation, flavin attachment, formylation, gamma-carboxylation, glutamylation, glycylation, glycosylation, glypiation, heme C attachment, hydroxylation, hypusine formation, iodination, isoprenylation, lipidation, lipoylation, malonylation, methylation, myristolylation, oxidation, palmitoylation, pegylation, phosphopantetheinylation, phosphorylation, prenylation, propionylation, retinylidene Schiff base formation, S-glutathionylation, S-nitrosylation, S-sulfenylation, selenation, succinylation, sulfination, ubiquitination, and C-terminal amidation. A post-translational modification includes modifications of the amino terminus and/or the carboxyl terminus of a peptide. Modifications of the terminal amino group include, but are not limited to, des-amino, N-lower alkyl, N-di-lower alkyl, and N-acyl modifications. Modifications of the terminal carboxy group include, but are not limited to, amide, lower alkyl amide, dialkyl amide, and lower alkyl ester modifications (e.g., wherein lower alkyl is C₁-C₄ alkyl). A post-translational modification also includes modifications, such as but not limited to those described above, of amino acids falling between the amino and carboxy termini. The term post-translational modification can also include peptide modifications that include one or more detectable labels.

As used herein, the term “binding agent” refers to a nucleic acid molecule, a peptide, a polypeptide, a protein, carbohydrate, or a small molecule that binds to, associates, unites with, recognizes, or combines with an analyte, e.g., a macromolecule or a component or feature of a macromolecule. A binding agent may form a covalent association or non-covalent association with the analyte, e.g., a macromolecule or component or feature of a macromolecule. A binding agent may also be a chimeric binding agent, composed of two or more types of molecules, such as a nucleic acid molecule-peptide chimeric binding agent or a carbohydrate-peptide chimeric binding agent. A binding agent may be a naturally occurring, synthetically produced, or recombinantly expressed molecule. A binding agent may bind to a single monomer or subunit of a macromolecule (e.g., a single amino acid of a peptide) or bind to a plurality of linked subunits of a macromolecule (e.g., a di-peptide, tri-peptide, or higher order peptide of a longer peptide, polypeptide, or protein molecule). A binding agent may bind to a linear molecule or a molecule having a three-dimensional structure (also referred to as conformation). For example, an antibody binding agent may bind to linear peptide, polypeptide, or protein, or bind to a conformational peptide, polypeptide, or protein. A binding agent may bind to an N-terminal peptide, a C-terminal peptide, or an intervening peptide of a peptide, polypeptide, or protein molecule. A binding agent may bind to an N-terminal amino acid, C-terminal amino acid, or an intervening amino acid of a peptide molecule. A binding agent may bind to an N-terminal or C-terminal diamino acid moiety. A binding agent may for example bind to a chemically modified or labeled amino acid over a non-modified or unlabeled amino acid. For example, a binding agent may for example bind to an amino acid that has been modified with an acetyl moiety, cbz moiety, guanyl moiety, amino guanidine moiety, dansyl moiety, phenylthiocarbamoyl (PTC) moiety, dinitrophenyl (DNP) moiety, sulfonyl nitrophenyl (SNP) moiety, diheterocyclic methanimine moiety, etc., over an amino acid that does not possess said moiety. A binding agent may bind to a post-translational modification of a polypeptide molecule. A binding agent may exhibit selective binding to a component or feature of an analyte, such as a macromolecule (e.g., a binding agent may selectively bind to one of the 20 possible natural amino acid residues and bind with very low affinity or not at all to the other 19 natural amino acid residues). A binding agent may exhibit less selective binding, where the binding agent is capable of binding a plurality of components or features of an analyte, such as a macromolecule (e.g., a binding agent may bind with similar affinity to two or more different amino acid residues). A binding agent may comprise a coding tag, which may be joined to the binding agent by a linker.

As used herein, the term “fluorophore” refers to a molecule which absorbs electromagnetic energy at one wavelength and re-emits energy at another wavelength. A fluorophore may be a molecule or part of a molecule including fluorescent dyes and proteins. Additionally, a fluorophore may be chemically, genetically, or otherwise connected or fused to another molecule to produce a molecule that has been “tagged” with the fluorophore.

As used herein, the term “linker” refers to one or more of a nucleotide, a nucleotide analog, an amino acid, a peptide, a polypeptide, or a non-nucleotide chemical moiety that is used to join two molecules. A linker may be used to join a binding agent with a coding tag, a recording tag with a polypeptide, a polypeptide with a solid support, a recording tag with a solid support, etc. In certain embodiments, a linker joins two molecules via enzymatic reaction or chemistry reaction (e.g., click chemistry).

As used herein, the term “proteome” can include the entire set of proteins, polypeptides, or peptides (including conjugates or complexes thereof) expressed by a genome, cell, tissue, or organism at a certain time, of any organism. In one aspect, it is the set of expressed proteins in a given type of cell or organism, at a given time, under defined conditions. Proteomics is the study of the proteome. For example, a “cellular proteome” may include the collection of proteins found in a particular cell type under a particular set of environmental conditions, such as exposure to hormone stimulation. An organism's complete proteome may include the complete set of proteins from all of the various cellular proteomes. A proteome may also include the collection of proteins in certain sub-cellular biological systems. For example, all of the proteins in a virus can be called a viral proteome. As used herein, the term “proteome” include subsets of a proteome, including but not limited to a kinome; a secretome; a receptome (e.g., GPCRome); an immunoproteome; a nutriproteome; a proteome subset defined by a post-translational modification (e.g., phosphorylation, ubiquitination, methylation, acetylation, glycosylation, oxidation, lipidation, and/or nitrosylation), such as a phosphoproteome (e.g., phosphotyrosine-proteome, tyrosine-kinome, and tyrosine-phosphatome), a glycoproteome, etc.; a proteome subset associated with a tissue or organ, a developmental stage, or a physiological or pathological condition; a proteome subset associated a cellular process, such as cell cycle, differentiation (or de-differentiation), cell death, senescence, cell migration, transformation, or metastasis; or any combination thereof. As used herein, the term “proteomics” refers to analysis of the proteome within cells, tissues, and bodily fluids, and the corresponding spatial distribution of the proteome within the cell and within tissues. Additionally, proteomics studies include the dynamic state of the proteome, continually changing in time as a function of biology and defined biological or chemical stimuli.

The terminal amino acid at one end of the peptide chain that has a free amino group is referred to herein as the “N-terminal amino acid” (NTAA). The terminal amino acid at the other end of the chain that has a free carboxyl group is referred to herein as the “C-terminal amino acid” (CTAA). An N-terminal diamino acid may comprise the N-terminal amino acid and the penultimate N-terminal amino acid. A C-terminal diamino acid is similarly defined for the C-terminus. The amino acids making up a peptide may be numbered in order, with the peptide being “n” amino acids in length. As used herein, NTAA is considered the n-amino acid (also referred to herein as the “n NTAA”). Using this nomenclature, the next amino acid is the n-1 amino acid, then the n-2 amino acid, and so on down the length of the peptide from the N-terminal end to C-terminal end. In certain embodiments, an NTAA, CTAA, or both may be functionalized with a chemical moiety.

As used herein, the term “barcode” refers to a nucleic acid molecule of about 2 to about 30 bases (e.g., 2, 3, 4, 5, 6, 7, 8, 9, 10, 11, 12, 13, 14, 15, 16, 17, 18, 19, 20, 21, 22, 23, 24, 25, 26, 27, 28, 29 or 30 bases) providing a unique identifier tag or origin information for a polypeptide, a binding agent, a set of binding agents from a binding cycle, a sample polypeptides, a set of samples, polypeptides within a compartment (e.g., droplet, bead, or separated location), polypeptides within a set of compartments, a fraction of polypeptides, a set of polypeptide fractions, a spatial region or set of spatial regions, a library of polypeptides, or a library of binding agents. A barcode can be an artificial sequence or a naturally occurring sequence. In certain embodiments, each barcode within a population of barcodes is different. In other embodiments, a portion of barcodes in a population of barcodes is different, e.g., at least about 10%, 15%, 20%, 25%, 30%, 35%, 40%, 45%, 50%, 55%, 60%, 65%, 70%, 75%, 80%, 85%, 90%, 95%, 97%, or 99% of the barcodes in a population of barcodes is different. A population of barcodes may be randomly generated or non-randomly generated. In certain embodiments, a population of barcodes are error correcting barcodes. Barcodes can be used to computationally deconvolute the multiplexed sequencing data and identify sequence reads derived from an individual polypeptide, sample, library, etc. A barcode can also be used for deconvolution of a collection of polypeptides that have been distributed into small compartments for enhanced mapping. For example, rather than mapping a peptide back to the proteome, the peptide is mapped back to its originating protein molecule or protein complex.

A “sample barcode”, also referred to as “sample tag” identifies from which sample a polypeptide derives.

As used herein, the term “coding tag” refers to a polynucleotide with any suitable length, e.g., a nucleic acid molecule of about 2 bases to about 100 bases, including any integer including 2 and 100 and in between, that comprises identifying information for its associated binding agent. A “coding tag” may also be made from a “sequenceable polymer” (see, e.g., Niu et al., 2013, Nat. Chem. 5:282-292; Roy et al., 2015, Nat. Commun. 6:7237; Lutz, 2015, Macromolecules 48:4759-4767; each of which are incorporated by reference in its entirety). A coding tag may comprise an encoder sequence, which is optionally flanked by one spacer on one side or optionally flanked by a spacer on each side. A coding tag may also be comprised of an optional UMI and/or an optional binding cycle-specific barcode. A coding tag may be single stranded or double stranded. A double stranded coding tag may comprise blunt ends, overhanging ends, or both. A coding tag may refer to the coding tag that is directly attached to a binding agent, to a complementary sequence hybridized to the coding tag directly attached to a binding agent (e.g., for double stranded coding tags), or to coding tag information present in an extended recording tag. In certain embodiments, a coding tag may further comprise a binding cycle specific spacer or barcode, a unique molecular identifier, a universal priming site, or any combination thereof.

As used herein, the term “encoder sequence” or “encoder barcode” refers to a nucleic acid molecule of about 2 bases to about 30 bases (e.g., 2, 3, 4, 5, 6, 7, 8, 9, 10, 11, 12, 13, 14, 15, 16, 17, 18, 19, 20, 21, 22, 23, 24, 25, 26, 27, 28, 29 or 30 bases) in length that provides identifying information for its associated binding agent. The encoder sequence may uniquely identify its associated binding agent. In certain embodiments, an encoder sequence is provides identifying information for its associated binding agent and for the binding cycle in which the binding agent is used. In other embodiments, an encoder sequence is combined with a separate binding cycle-specific barcode within a coding tag. Alternatively, the encoder sequence may identify its associated binding agent as belonging to a member of a set of two or more different binding agents. In some embodiments, this level of identification is sufficient for the purposes of analysis. For example, in some embodiments involving a binding agent that binds to an amino acid, it may be sufficient to know that a peptide comprises one of two possible amino acids at a particular position, rather than definitively identify the amino acid residue at that position. In another example, a common encoder sequence is used for polyclonal antibodies, which comprises a mixture of antibodies that recognize more than one epitope of a protein target, and have varying specificities. In other embodiments, where an encoder sequence identifies a set of possible binding agents, a sequential decoding approach can be used to produce unique identification of each binding agent. This is accomplished by varying encoder sequences for a given binding agent in repeated cycles of binding (see, Gunderson et al., 2004, Genome Res. 14:870-7). The partially identifying coding tag information from each binding cycle, when combined with coding information from other cycles, produces a unique identifier for the binding agent, e.g., the particular combination of coding tags rather than an individual coding tag (or encoder sequence) provides the uniquely identifying information for the binding agent. Preferably, the encoder sequences within a library of binding agents possess the same or a similar number of bases.

As used herein the term “binding cycle specific tag”, “binding cycle specific barcode”, or “binding cycle specific sequence” refers to a unique sequence used to identify a library of binding agents used within a particular binding cycle. A binding cycle specific tag may comprise about 2 bases to about 8 bases (e.g., 2, 3, 4, 5, 6, 7, or 8 bases) in length. A binding cycle specific tag may be incorporated within a binding agent's coding tag as part of a spacer sequence, part of an encoder sequence, part of a UMI, or as a separate component within the coding tag.

As used herein, the term “spacer” (Sp) refers to a nucleic acid molecule of about 1 base to about 20 bases (e.g., 1, 2, 3, 4, 5, 6, 7, 8, 9, 10, 11, 12, 13, 14, 15, 16, 17, 18, 19, or 20 bases) in length that is present on a terminus of a recording tag or coding tag. In certain embodiments, a spacer sequence flanks an encoder sequence of a coding tag on one end or both ends. Following binding of a binding agent to a polypeptide, annealing between complementary spacer sequences on their associated coding tag and recording tag, respectively, allows transfer of binding information through a primer extension reaction or ligation to the recording tag. Sp′ refers to spacer sequence complementary to Sp. Preferably, spacer sequences within a library of binding agents possess the same number of bases. A common (shared or identical) spacer may be used in a library of binding agents. A spacer sequence may have a “cycle specific” sequence in order to track binding agents used in a particular binding cycle. The spacer sequence (Sp) can be constant across all binding cycles, be specific for a particular class of polypeptides, or be binding cycle number specific. Polypeptide class-specific spacers permit annealing of a cognate binding agent's coding tag information present in an extended recording tag from a completed binding/extension cycle to the coding tag of another binding agent recognizing the same class of polypeptides in a subsequent binding cycle via the class-specific spacers. Only the sequential binding of correct cognate pairs results in interacting spacer elements and effective primer extension. A spacer sequence may comprise sufficient number of bases to anneal to a complementary spacer sequence in a recording tag to initiate a primer extension (also referred to as polymerase extension) reaction, or provide a “splint” for a ligation reaction, or mediate a “sticky end” ligation reaction. A spacer sequence may comprise a fewer number of bases than the encoder sequence within a coding tag.

As used herein, the term “recording tag” refers to a moiety, e.g., a chemical coupling moiety, a nucleic acid molecule, or a sequenceable polymer molecule (see, e.g., Niu et al., 2013, Nat. Chem. 5:282-292; Roy et al., 2015, Nat. Commun. 6:7237; Lutz, 2015, Macromolecules 48:4759-4767; each of which are incorporated by reference in its entirety) to which identifying information of a coding tag can be transferred, or from which identifying information about the macromolecule (e.g., UMI information) associated with the recording tag can be transferred to the coding tag. Identifying information can comprise any information characterizing a molecule such as information pertaining to sample, fraction, partition, spatial location, interacting neighboring molecule(s), cycle number, etc. Additionally, the presence of UMI information can also be classified as identifying information. In certain embodiments, after a binding agent binds to a polypeptide, information from a coding tag linked to a binding agent can be transferred to the recording tag associated with the polypeptide while the binding agent is bound to the polypeptide. In other embodiments, after a binding agent binds to a polypeptide, information from a recording tag associated with the polypeptide can be transferred to the coding tag linked to the binding agent while the binding agent is bound to the polypeptide. A recoding tag may be directly linked to a macromolecule, e.g., a polypeptide, linked to a macromolecule, e.g., a polypeptide, via a multifunctional linker, or associated with a macromolecule, e.g., a polypeptide, by virtue of its proximity (or co-localization) on a solid support. A recording tag may be linked via its 5′ end or 3′ end or at an internal site, if the linkage is compatible with the method used to transfer coding tag information to the recording tag or vice versa. A recording tag may further comprise other functional components, e.g., a universal priming site, unique molecular identifier, a barcode (e.g., a sample barcode, a fraction barcode, spatial barcode, a compartment tag, etc.), a spacer sequence that is complementary to a spacer sequence of a coding tag, or any combination thereof. The spacer sequence of a recording tag is preferably at the 3′-end of the recording tag in embodiments where polymerase extension is used to transfer coding tag information to the recording tag.

As used herein, the term “primer extension”, also referred to as “polymerase extension”, refers to a reaction catalyzed by a nucleic acid polymerase (e.g., DNA polymerase) whereby a nucleic acid molecule (e.g., oligonucleotide primer, spacer sequence) that anneals to a complementary strand is extended by the polymerase, using the complementary strand as template.

As used herein, the term “unique molecular identifier” or “UMI” refers to a nucleic acid molecule of about 3 to about 40 bases (3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 bases in length providing a unique identifier tag for each polypeptide or binding agent to which the UMI is linked. A polypeptide UMI can be used to computationally deconvolute sequencing data from a plurality of extended recording tags to identify extended recording tags that originated from an individual polypeptide. A polypeptide UMI can be used to accurately count originating polypeptide molecules by collapsing NGS reads to unique UMIs. A binding agent UMI can be used to identify each individual molecular binding agent that binds to a particular polypeptide. For example, a UMI can be used to identify the number of individual binding events for a binding agent specific for a single amino acid that occurs for a particular peptide molecule.

As used herein, the term “universal priming site” or “universal primer” or “universal priming sequence” refers to a nucleic acid molecule, which may be used for library amplification and/or for sequencing reactions. A universal priming site may include, but is not limited to, a priming site (primer sequence) for PCR amplification, flow cell adaptor sequences that anneal to complementary oligonucleotides on flow cell surfaces enabling bridge amplification in some next generation sequencing platforms, a sequencing priming site, or a combination thereof. Universal priming sites can be used for other types of amplification, including those commonly used in conjunction with next generation digital sequencing. For example, extended recording tag molecules may be circularized and a universal priming site used for rolling circle amplification to form DNA nanoballs that can be used as sequencing templates (Drmanac et al., 2009, Science 327:78-81). Alternatively, recording tag molecules may be circularized and sequenced directly by polymerase extension from universal priming sites (Korlach et al., 2008, Proc. Natl. Acad. Sci. 105:1176-1181). The term “forward” when used in context with a “universal priming site” or “universal primer” may also be referred to as “5′” or “sense”. The term “reverse” when used in context with a “universal priming site” or “universal primer” may also be referred to as “3′” or “antisense”.

As used herein, the term “extended recording tag” refers to a recording tag to which information of at least one binding agent's coding tag (or its complementary sequence) has been transferred following binding of the binding agent to a macromolecule, e.g., a polypeptide. Information of the coding tag may be transferred to the recording tag directly (e.g., ligation) or indirectly (e.g., primer extension). Information of a coding tag may be transferred to the recording tag enzymatically or chemically. An extended recording tag may comprise binding agent information of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, 100, 125, 150, 175, 200 or more coding tags. The base sequence of an extended recording tag may reflect the temporal and sequential order of binding of the binding agents identified by their coding tags, may reflect a partial sequential order of binding of the binding agents identified by the coding tags, or may not reflect any order of binding of the binding agents identified by the coding tags. In certain embodiments, the coding tag information present in the extended recording tag represents with at least 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% identity the polypeptide sequence being analyzed. In certain embodiments where the extended recording tag does not represent the polypeptide sequence being analyzed with 100% identity, errors may be due to off-target binding by a binding agent, or to a “missed” binding cycle (e.g., because a binding agent fails to bind to a polypeptide during a binding cycle, because of a failed primer extension reaction), or both.

As used herein, the term “solid support”, “solid surface”, or “solid substrate”, or “sequencing substrate”, or “substrate” refers to any solid material, including porous and non-porous materials, to which a polypeptide can be associated directly or indirectly, by any means known in the art, including covalent and non-covalent interactions, or any combination thereof. A solid support may be two-dimensional (e.g., planar surface) or three-dimensional (e.g., gel matrix or bead). A solid support can be any support surface including, but not limited to, a bead, a microbead, an array, a glass surface, a silicon surface, a plastic surface, a filter, a membrane, nylon, a silicon wafer chip, a flow through chip, a flow cell, a biochip including signal transducing electronics, a channel, a microtiter well, an ELISA plate, a spinning interferometry disc, a nitrocellulose membrane, a nitrocellulose-based polymer surface, a polymer matrix, a nanoparticle, or a microsphere. Materials for a solid support include but are not limited to acrylamide, agarose, cellulose, nitrocellulose, glass, gold, quartz, polystyrene, polyethylene vinyl acetate, polypropylene, polymethacrylate, polyethylene, polyethylene oxide, polysilicates, polycarbonates, Teflon, fluorocarbons, nylon, silicon rubber, polyanhydrides, polyglycolic acid, polyactic acid, polyorthoesters, functionalized silane, polypropylfumerate, collagen, glycosaminoglycans, polyamino acids, dextran, or any combination thereof. Solid supports further include thin film, membrane, bottles, dishes, fibers, woven fibers, shaped polymers such as tubes, particles, beads, microspheres, microparticles, or any combination thereof. For example, when solid surface is a bead, the bead can include, but is not limited to, a ceramic bead, polystyrene bead, a polymer bead, a methylstyrene bead, an agarose bead, an acrylamide bead, a solid core bead, a porous bead, a paramagnetic bead, a glass bead, or a controlled pore bead. A bead may be spherical or an irregularly shaped. A bead or support may be porous. A bead's size may range from nanometers, e.g., 100 nm, to millimeters, e.g., 1 mm. In certain embodiments, beads range in size from about 0.2 micron to about 200 microns, or from about 0.5 micron to about 5 micron. In some embodiments, beads can be about 1, 1.5, 2, 2.5, 2.8, 3, 3.5, 4, 4.5, 5, 5.5, 6, 6.5, 7, 7.5, 8, 8.5, 9, 9.5, 10, 10.5, 15, or 20 μm in diameter. In certain embodiments, “a bead” solid support may refer to an individual bead or a plurality of beads. In some embodiments, the solid surface is a nanoparticle. In certain embodiments, the nanoparticles range in size from about 1 nm to about 500 nm in diameter, for example, between about 1 nm and about 20 nm, between about 1 nm and about 50 nm, between about 1 nm and about 100 nm, between about 10 nm and about 50 nm, between about 10 nm and about 100 nm, between about 10 nm and about 200 nm, between about 50 nm and about 100 nm, between about 50 nm and about 150, between about 50 nm and about 200 nm, between about 100 nm and about 200 nm, or between about 200 nm and about 500 nm in diameter. In some embodiments, the nanoparticles can be about 10 nm, about 50 nm, about 100 nm, about 150 nm, about 200 nm, about 300 nm, or about 500 nm in diameter. In some embodiments, the nanoparticles are less than about 200 nm in diameter.

As used herein, the term “nucleic acid molecule” or “polynucleotide” refers to a single- or double-stranded polynucleotide containing deoxyribonucleotides or ribonucleotides that are linked by 3′-5′ phosphodiester bonds, as well as polynucleotide analogs. A nucleic acid molecule includes, but is not limited to, DNA, RNA, and cDNA. A polynucleotide analog may possess a backbone other than a standard phosphodiester linkage found in natural polynucleotides and, optionally, a modified sugar moiety or moieties other than ribose or deoxyribose. Polynucleotide analogs contain bases capable of hydrogen bonding by Watson-Crick base pairing to standard polynucleotide bases, where the analog backbone presents the bases in a manner to permit such hydrogen bonding in a sequence-specific fashion between the oligonucleotide analog molecule and bases in a standard polynucleotide. Examples of polynucleotide analogs include, but are not limited to xeno nucleic acid (XNA), bridged nucleic acid (BNA), glycol nucleic acid (GNA), peptide nucleic acids (PNAs), gPNAs, morpholino polynucleotides, locked nucleic acids (LNAs), threose nucleic acid (TNA), 2′-O-Methyl polynucleotides, 2′-O-alkyl ribosyl substituted polynucleotides, phosphorothioate polynucleotides, and boronophosphate polynucleotides. A polynucleotide analog may possess purine or pyrimidine analogs, including for example, 7-deaza purine analogs, 8-halopurine analogs, 5-halopyrimidine analogs, or universal base analogs that can pair with any base, including hypoxanthine, nitroazoles, isocarbostyril analogues, azole carboxamides, and aromatic triazole analogues, or base analogs with additional functionality, such as a biotin moiety for affinity binding. In some embodiments, the nucleic acid molecule or oligonucleotide is a modified oligonucleotide. In some embodiments, the nucleic acid molecule or oligonucleotide is a DNA with pseudo-complementary bases, a DNA with protected bases, an RNA molecule, a BNA molecule, an XNA molecule, a LNA molecule, a PNA molecule, a gPNA molecule, or a morpholino DNA, or a combination thereof. In some embodiments, the nucleic acid molecule or oligonucleotide is backbone modified, sugar modified, or nucleobase modified. In some embodiments, the nucleic acid molecule or oligonucleotide has nucleobase protecting groups such as Alloc, electrophilic protecting groups such as thiranes, acetyl protecting groups, nitrobenzyl protecting groups, sulfonate protecting groups, or traditional base-labile protecting groups.

As used herein, “nucleic acid sequencing” means the determination of the order of nucleotides in a nucleic acid molecule or a sample of nucleic acid molecules.

As used herein, “next generation sequencing” refers to high-throughput sequencing methods that allow the sequencing of millions to billions of molecules in parallel. Examples of next generation sequencing methods include sequencing by synthesis, sequencing by ligation, sequencing by hybridization, polony sequencing, ion semiconductor sequencing, and pyrosequencing. By attaching primers to a solid substrate and a complementary sequence to a nucleic acid molecule, a nucleic acid molecule can be hybridized to the solid substrate via the primer and then multiple copies can be generated in a discrete area on the solid substrate by using polymerase to amplify (these groupings are sometimes referred to as polymerase colonies or polonies). Consequently, during the sequencing process, a nucleotide at a particular position can be sequenced multiple times (e.g., hundreds or thousands of times)—this depth of coverage is referred to as “deep sequencing.” Examples of high throughput nucleic acid sequencing technology include platforms provided by Illumina, BGI, Qiagen, Thermo-Fisher, and Roche, including formats such as parallel bead arrays, sequencing by synthesis, sequencing by ligation, capillary electrophoresis, electronic microchips, “biochips,” microarrays, parallel microchips, and single-molecule arrays, as reviewed by Service (Science 311:1544-1546, 2006).

As used herein, “single molecule sequencing” or “third generation sequencing” refers to next-generation sequencing methods wherein reads from single molecule sequencing instruments are generated by sequencing of a single molecule of DNA. Unlike next generation sequencing methods that rely on amplification to clone many DNA molecules in parallel for sequencing in a phased approach, single molecule sequencing interrogates single molecules of DNA and does not require amplification or synchronization. Single molecule sequencing includes methods that need to pause the sequencing reaction after each base incorporation (‘wash-and-scan’ cycle) and methods which do not need to halt between read steps. Examples of single molecule sequencing methods include single molecule real-time sequencing (Pacific Biosciences), nanopore-based sequencing (Oxford Nanopore), duplex interrupted nanopore sequencing, and direct imaging of DNA using advanced microscopy.

As used herein, “analyzing” a macromolecule, means to identify, quantify, characterize, distinguish, or a combination thereof, all or a portion of the components of the macromolecule. For example, analyzing a peptide, polypeptide, or protein includes determining all or a portion of the amino acid sequence (contiguous or non-continuous) of the peptide. Analyzing a macromolecule also includes partial identification of a component of the macromolecule. For example, partial identification of amino acids in the macromolecule protein sequence can identify an amino acid in the protein as belonging to a subset of possible amino acids. Analysis typically begins with analysis of the n-NTAA, and then proceeds to the next amino acid of the peptide (i.e., n-1, n-2, n-3, and so forth). This is accomplished by cleavage of the n^{th} NTAA, thereby converting the (n-1)^{th} amino acid of the peptide to an N-terminal amino acid (referred to herein as the “(n-1)^{th} NTAA”). Analyzing the peptide may also include determining the presence and frequency of post-translational modifications on the peptide, which may or may not include information regarding the sequential order of the post-translational modifications on the peptide. Analyzing the peptide may also include determining the presence and frequency of epitopes in the peptide, which may or may not include information regarding the sequential order or location of the epitopes within the peptide. Analyzing the peptide may include combining different types of analysis, for example obtaining epitope information, amino acid sequence information, post-translational modification information, or any combination thereof.

As used herein, the term “compartment” refers to a physical area or volume that separates or isolates a subset of macromolecules from a sample of macromolecules. For example, a compartment may separate an individual cell from other cells, or a subset of a sample's proteome from the rest of the sample's proteome. A compartment may be an aqueous compartment (e.g., microfluidic droplet), a solid compartment (e.g., picotiter well or microtiter well on a plate, tube, vial, gel bead), or a separated region on a surface. A compartment may comprise one or more beads to which macromolecules may be immobilized.

As used herein, the term “compartment tag” or “compartment barcode” refers to a single or double stranded nucleic acid molecule of about 4 bases to about 100 bases (including 4 bases, 100 bases, and any integer between) that comprises identifying information for the constituents (e.g., a single cell's proteome), within one or more compartments (e.g., microfluidic droplet). A compartment barcode identifies a subset of macromolecules in a sample, e.g., a subset of protein sample, that have been separated into the same physical compartment or group of compartments from a plurality (e.g., millions to billions) of compartments. Thus, a compartment tag can be used to distinguish constituents derived from one or more compartments having the same compartment tag from those in another compartment having a different compartment tag, even after the constituents are pooled together. By labeling the proteins and/or peptides within each compartment or within a group of two or more compartments with a unique compartment tag, peptides derived from the same protein, protein complex, or cell within an individual compartment or group of compartments can be identified. A compartment tag comprises a barcode, which is optionally flanked by a spacer sequence on one or both sides, and an optional universal primer. The spacer sequence can be complementary to the spacer sequence of a recording tag, enabling transfer of compartment tag information to the recording tag. A compartment tag may also comprise a universal priming site, a unique molecular identifier (for providing identifying information for the peptide attached thereto), or both, particularly for embodiments where a compartment tag comprises a recording tag to be used in downstream peptide analysis methods described herein. A compartment tag can comprise a functional moiety (e.g., a click chemistry moiety, aldehyde, NHS, mTet, alkyne, etc.) for coupling to a peptide. Alternatively, a compartment tag can comprise a peptide comprising a recognition sequence for a protein ligase to allow ligation of the compartment tag to a peptide of interest. A compartment can comprise a single compartment tag, a plurality of identical compartment tags save for an optional UMI sequence, or two or more different compartment tags. In certain embodiments each compartment comprises a unique compartment tag (one-to-one mapping). In other embodiments, multiple compartments from a larger population of compartments comprise the same compartment tag (many-to-one mapping). A compartment tag may be joined to a solid support within a compartment (e.g., bead) or joined to the surface of the compartment itself (e.g., surface of a picotiter well). Alternatively, a compartment tag may be free in solution within a compartment.

As used herein, the term “partition” refers to an assignment of a unique barcode to a subpopulation of macromolecules from a population of macromolecules within a sample. In certain embodiments, partitioning may be achieved by distributing macromolecules into compartments. A partition may be comprised of the macromolecules within a single compartment or the macromolecules within multiple compartments from a population of compartments.

As used herein, a “partition tag” or “partition barcode” refers to a single or double stranded nucleic acid molecule of about 4 bases to about 100 bases (including 4 bases, 100 bases, and any integer between) that comprises identifying information for a partition. In certain embodiments, a partition tag for a macromolecule refers to identical compartment tags arising from the partitioning of macromolecules into compartment(s) labeled with the same barcode.

As used herein, the term “fraction” refers to a subset of macromolecules (e.g., proteins) within a sample that have been sorted from the rest of the sample or organelles using physical or chemical separation methods, such as fractionating by size, hydrophobicity, isoelectric point, affinity, and so on. Separation methods include HPLC separation, gel separation, affinity separation, cellular fractionation, cellular organelle fractionation, tissue fractionation, etc. Physical properties such as fluid flow, magnetism, electrical current, mass, density, or the like can also be used for separation.

As used herein, the term “fraction barcode” refers to a single or double stranded nucleic acid molecule of about 4 bases to about 100 bases (including 4 bases, 100 bases, and any integer therebetween) that comprises identifying information for the macromolecules within a fraction.

The term “about” as used herein refers to the usual error range for the respective value readily known to the skilled person in this technical field. Reference to “about” a value or parameter herein includes (and describes) embodiments that are directed to that value or parameter per se. For example, description referring to “about X” includes description of “X.

The term “antibody” herein is used in the broadest sense and includes polyclonal and monoclonal antibodies, including intact antibodies and functional (antigen-binding) antibody fragments, including fragment antigen binding (Fab) fragments, F(ab′)₂ fragments, Fab′ fragments, Fv fragments, recombinant IgG (rIgG) fragments, single chain antibody fragments, including single chain variable fragments (scFv), and single domain antibodies (e.g., sdAb, sdFv, nanobody) fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific, antibodies, diabodies, triabodies, and tetrabodies, tandem di-scFv, tandem tri-scFv. Unless otherwise stated, the term “antibody” should be understood to encompass functional antibody fragments thereof. The term also encompasses intact or full-length antibodies, including antibodies of any class or sub-class, including IgG and sub-classes thereof, IgM, IgE, IgA, and IgD.

An “individual” or “subject” includes a mammal. Mammals include, but are not limited to, domesticated animals (e.g., cows, sheep, cats, dogs, and horses), primates (e.g., humans and non-human primates such as monkeys), rabbits, and rodents (e.g., mice and rats). An “individual” or “subject” may include birds such as chickens, vertebrates such as fish and mammals such as mice, rats, rabbits, cats, dogs, pigs, cows, ox, sheep, goats, horses, monkeys and other non-human primates. In certain embodiments, the individual or subject is a human.

As used herein, the term “sample” refers to anything which may contain an analyte for which an analyte assay is desired. As used herein, a “sample” can be a solution, a suspension, liquid, powder, a paste, aqueous, non-aqueous or any combination thereof. The sample may be a biological sample, such as a biological fluid or a biological tissue. Examples of biological fluids include urine, blood, plasma, serum, saliva, semen, stool, sputum, cerebral spinal fluid, tears, mucus, amniotic fluid or the like. Biological tissues are aggregate of cells, usually of a particular kind together with their intercellular substance that form one of the structural materials of a human, animal, plant, bacterial, fungal or viral structure, including connective, epithelium, muscle and nerve tissues. Examples of biological tissues also include organs, tumors, lymph nodes, arteries and individual cell(s).

In some embodiments, the sample is a biological sample. A biological sample of the present disclosure encompasses a sample in the form of a solution, a suspension, a liquid, a powder, a paste, an aqueous sample, or a non-aqueous sample. As used herein, a “biological sample” includes any sample obtained from a living or viral (or prion) source or other source of macromolecules and biomolecules, and includes any cell type or tissue of a subject from which nucleic acid, protein and/or other macromolecule can be obtained. The biological sample can be a sample obtained directly from a biological source or a sample that is processed. For example, isolated nucleic acids that are amplified constitute a biological sample. Biological samples include, but are not limited to, body fluids, such as blood, plasma, serum, cerebrospinal fluid, synovial fluid, urine and sweat, tissue and organ samples from animals and plants and processed samples derived therefrom. In some embodiments, the sample can be derived from a tissue or a body fluid, for example, a connective, epithelium, muscle or nerve tissue; a tissue selected from the group consisting of brain, lung, liver, spleen, bone marrow, thymus, heart, lymph, blood, bone, cartilage, pancreas, kidney, gall bladder, stomach, intestine, testis, ovary, uterus, rectum, nervous system, gland, and internal blood vessels; or a body fluid selected from the group consisting of blood, urine, saliva, bone marrow, sperm, an ascitic fluid, and subfractions thereof, e.g., serum or plasma.

The terms “level” or “levels” are used to refer to the presence and/or amount of a target, e.g., a substance or an organism that is part of the etiology of a disease or disorder, and can be determined qualitatively or quantitatively. A “qualitative” change in the target level refers to the appearance or disappearance of a target that is not detectable or is present in samples obtained from normal controls. A “quantitative” change in the levels of one or more targets refers to a measurable increase or decrease in the target levels when compared to a healthy control.

It is understood that aspects and embodiments of the invention described herein include “consisting” and/or “consisting essentially of” aspects and embodiments.

Throughout this disclosure, various aspects of this invention are presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Other objects, advantages and features of the present invention will become apparent from the following specification taken in conjunction with the accompanying drawings.

### I. Apparatus for Automated Treatment of Samples

Provided herein is an apparatus for preparing or treating macromolecules (e.g., peptides, polypeptides, and proteins). In some embodiments, the macromolecules are immobilized, directly or indirectly via a linker, on a support. In some embodiments, the macromolecules for treatment using the apparatus are polypeptides or peptides immobilized on a substrate or support, e.g., a solid or porous substrate or support. In some embodiments, the apparatus is used to carry out one or more steps of a macromolecule analysis assay (e.g., a polypeptide analysis assay), such as any of the steps of the methods described herein, in an automated manner. The macromolecules analysis assay may include a cyclic process for treating the sample, wherein the process includes various repeated steps. The provided apparatus automates at least some of the repeated steps of the assay such that real-time input and control from a user is reduced. The apparatus may reduce the amount of time required from a user to perform the macromolecule analysis assay compared to a manual method performed without the apparatus. In some cases, the macromolecule analysis assay comprises nucleic acid encoding of molecule recognition events. In some cases, the provided apparatus is for use in treating, preparing, and/or modifying a macromolecule from a sample for sequencing and/or other analysis that employs barcoding. In some cases, the use of the apparatus for the treatment and/or preparation of the macromolecules enables downstream analysis of the sequence of single individual peptides, polypeptides, or proteins. The apparatus and automated treatment may be used to treat a plurality of samples simultaneously. In an exemplary workflow for analysis of the polypeptide analytes, a large collection of polypeptides (e.g., 50 million-1 billion) or more can be treated and analyzed using the automated methods and/or apparatus provided herein. In some embodiments, the apparatus is configured to integrate performing any combinations of the following: enzymatic reaction, an aqueous-phase biochemical reaction, and/or an organic reaction.

In some embodiments, the apparatus is for preparative procedures for treating the macromolecules in the sample for single-molecule analysis. In some particular cases, the apparatus is not used to observe a detectable signal that indicates the sequence of the macromolecule. In some cases, the readout from the macromolecule analysis assay is analyzed using a separate apparatus or instrument. In some particular embodiments, the apparatus is not configured for sensing single molecules in a sample. For example, in some embodiment, the apparatus does not comprise a single analyte sensor, wherein said sensor comprises an analyte-responsive surface. In some embodiment, the apparatus does not treat or process samples on a slide (e.g. a planar sample deposited on a planar surface such as a glass slide).

In some embodiments, the apparatus can be used to deliver sample(s) or be loaded with sample(s) in an automated manner. The sample(s) can be prepared for analysis appropriately before loading on to the apparatus, including digestion, chemical treatments, attachment of a protein sample with DNA tags to generate a peptide-DNA chimera, etc.). In some embodiments, one or more sample is provided to the apparatus and loaded by the apparatus to the sample container in an automated fashion. The sample container may comprise a support for attaching the sample, e.g., for attaching to peptide-DNA chimeras (see e.g., U.S. provisional patent application No. 62/840,675, filed on Apr. 30, 2019 and International Application No.: PCT/US2020/027840, filed on Apr. 10, 2020). In some embodiments, the sample(s) is provided from a sample-providing cartridge, which can be formatted for automatic loading into the sample container. In some embodiments, the apparatus may be designed and equipped with mechanics and features for automated sample loading, such as for mechanical engagement with the sample-providing cartridge. In some such cases, the sample-providing cartridge can be provided to the apparatus in the same or a different location than the reagent reservoirs.

The apparatus can be used for automating various processes by utilizing appropriate reagents in a supply system. In some embodiments, the apparatus can be used for automating various cyclic processes. In some cases, the automated processes may include setting and/or controlling cycling reaction temperatures for treating the sample in the sample containers. In some cases, the automated processes may include delivery of various reagents to the samples and performing washes. In some embodiments, appropriate control programs can be used with the provided apparatus. In some embodiments, appropriate reaction supports can be used with the apparatus. In some embodiments, additional steps may be performed using the apparatus to prepare the sample for the macromolecule analysis assay or to further process the sample after the macromolecule analysis assay. For example, the apparatus may be configured and used for an amplification reaction, thereby removing the need for a separate thermocycler and other instruments.

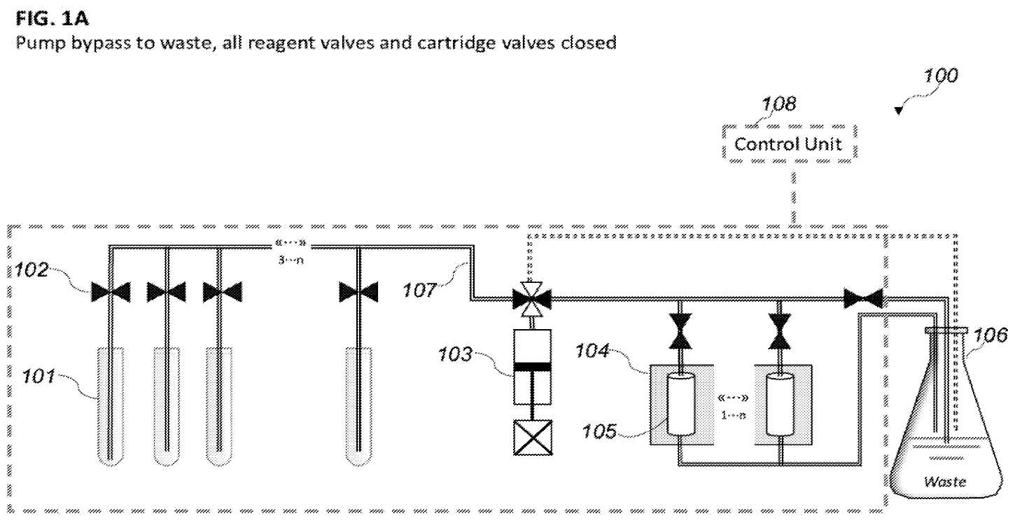

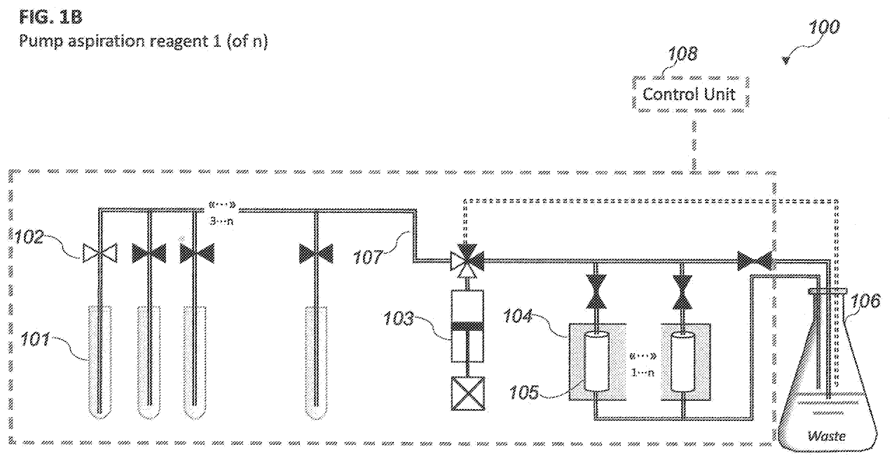

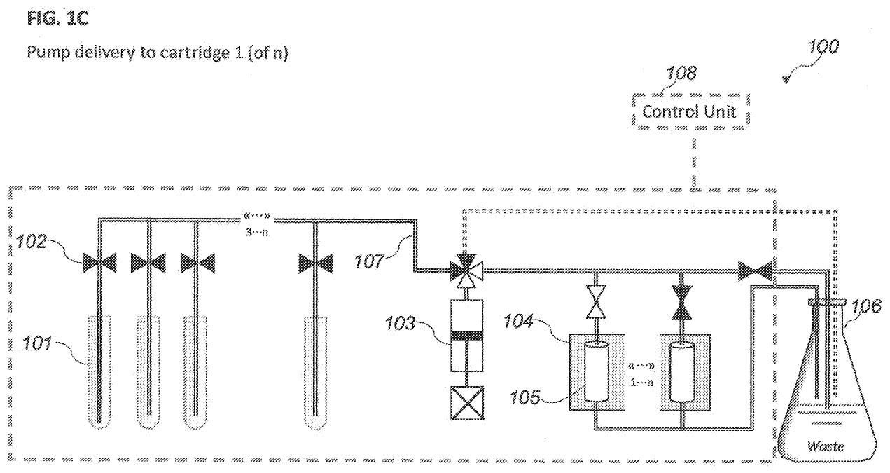

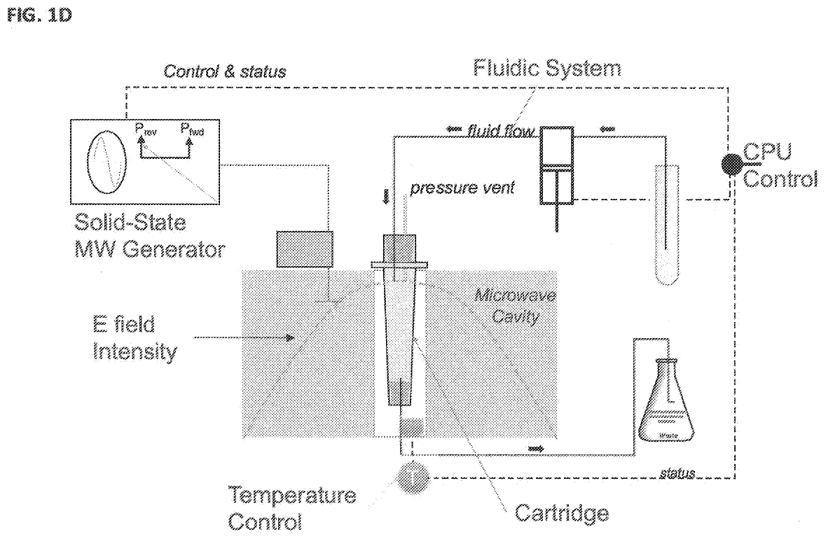

The apparatus includes one or more reagent reservoirs for containing a respective reagent. In some aspects, the apparatus includes a holder or space configured for holding said reagent reservoir(s). For example, the exemplary apparatus as shown in FIG. 1A-IC includes n number of reagent reservoirs 101. In some embodiments, one or more of the reagent reservoirs are subject to temperature control. In some examples, the reagent reservoirs may contain any or all of the following: buffers, wash buffers, polypeptides, nucleic acids, binding agents, enzymes, chemical reagents for modifying an amino acid, chemical reagents for cleaving one or more amino acids, enzymatic reagents for cleaving one or more amino acids, reagents for a ligation reaction, reagents for a polymerase-mediated reaction, or any combinations thereof.

The apparatus includes one or more sample containers and a temperature controlled unit which serves as a holder or space configured for holding the sample container(s) (e.g., cartridges). In some preferred embodiments, the apparatus is configured to hold a plurality of sample containers. For example, the exemplary apparatus as shown in FIG. 1A-1C includes n number of sample containers 105 contained in a temperature controlled unit 104. In some embodiments, the sample container is or comprises a cartridge comprising a filter means or a frit for retaining the sample while allowing flow-through of other materials (e.g. liquids or buffers).

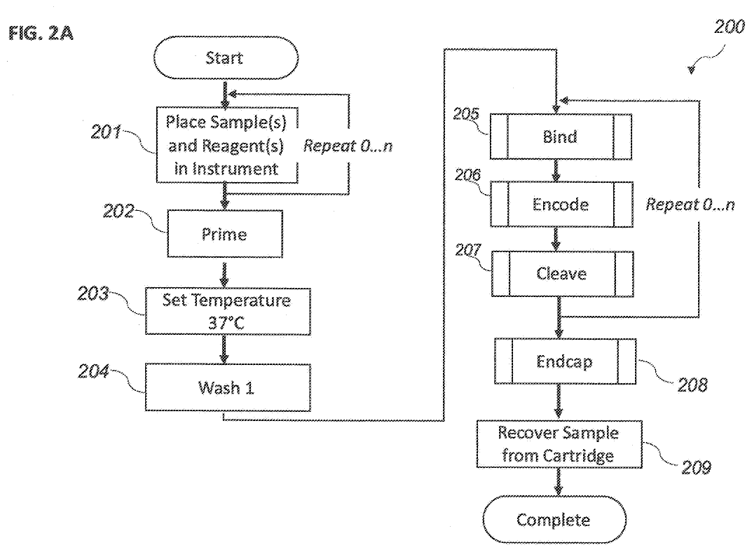

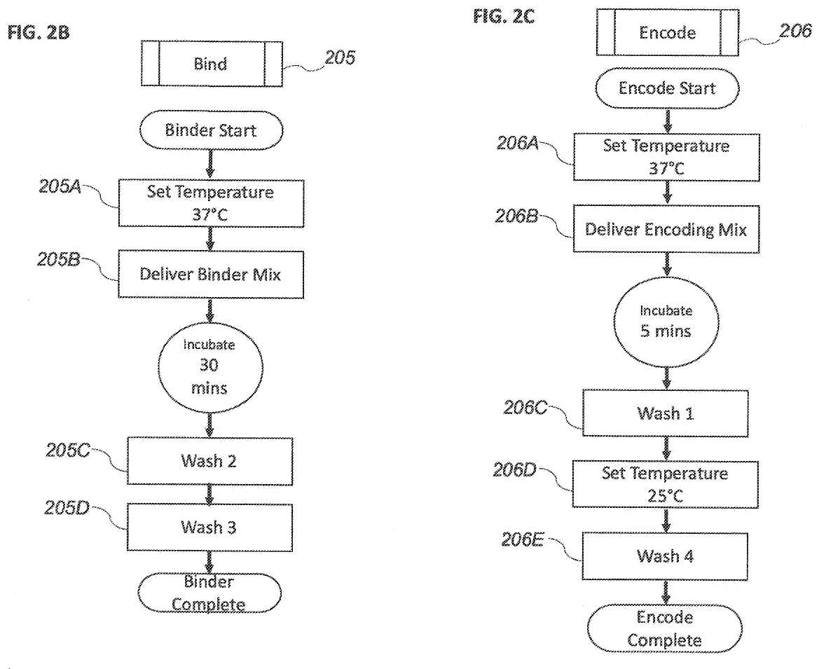

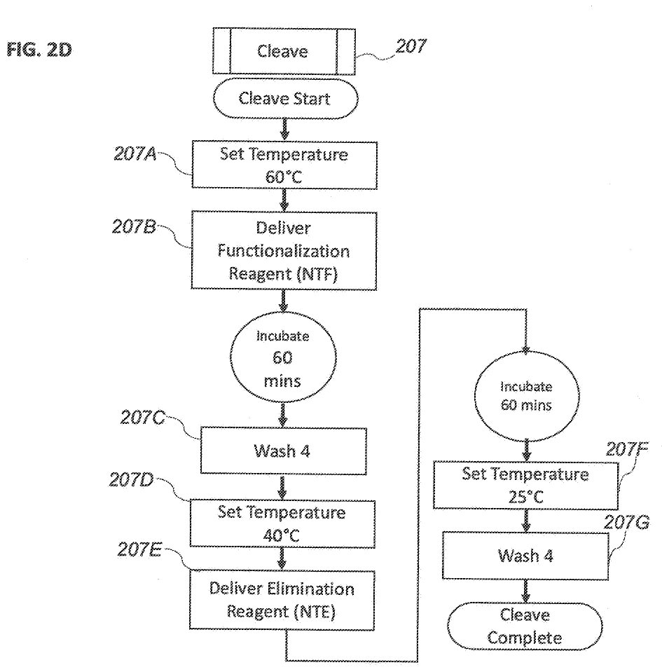

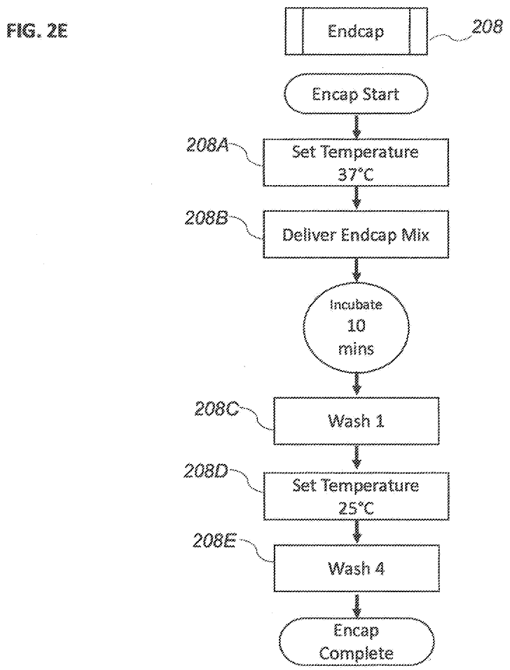

In some embodiments, one or more aspects of the apparatus is controlled by a control unit. For example, FIG. 1A-1C depicts a control system 108. In some cases, the control system also receives feedback from various components of the system. In some embodiments, the control system is in communication with one or more valves, one or more pumps, temperature controlled unit(s), and/or one or more sample containers. In some examples, a control unit is used to carry out one or more steps of a process as depicted in FIG. 2A-2C. In some aspects, the control unit is used to automate and/or control the temperature of the sample container(s). In some embodiments, the control unit is used to automate and/or control the temperature of the reagent reservoir(s). In some aspects, the control unit is used to automate and/or control the flow of liquids in the apparatus, (e.g., presence and absence of flow, position of a valve, direction of flow and/or flowrate, etc.). In some aspects, the control unit is used to automate and/or control the positioning of the valve(s) 102. In some cases, the control unit is used to automate and/or control and/or delivery of said one or more reagent(s) to said sample container(s) via control of a pump 103.

In some embodiments, the temperature of the sample container(s) subjected to temperature control and the temperature of the reagent reservoir(s) subjected to temperature control are individually controlled by the control unit. In some cases, the sample container(s) subjected to temperature control and the reagent reservoir(s) subjected to temperature control are housed in separate thermal blocks. In some cases, the sample container(s) subjected to temperature control and the reagent reservoir(s) subjected to temperature control are housed in the same thermal block.

In some embodiments, the apparatus includes a plurality of valves connected in a supply line having an upstream end and a downstream end, wherein at least one or each of said valves is positionable to provide alternate flow paths therethrough. In some embodiments, the reagent reservoirs are fluidically connected to said sample container(s). In some cases, the fluidic connection between the reagent reservoirs and sample containers is continuous. In some cases, the fluidic connection between the reagent reservoirs and sample containers is discontinuous or not completely continuous. In some embodiments, a closed system is formed from the reagent reservoirs to the sample containers. In some cases, the system is closed from input (e.g., from the reagent container) to waste. In some embodiments, one supply line connects a single reagent reservoir to a single sample container or to multiple sample containers. In some cases, one supply line connects multiple reagent reservoirs to multiple sample containers.

In an exemplary apparatus 100, a sample is loaded into a sample container (e.g. cartridge) and the cartridge is then placed in the instrument. In some embodiments, the sample comprises polypeptides prepared prior to 201, e.g. joining macromolecules in the sample to a solid support, joining macromolecules to a nucleic acid (e.g. a recording tag), digested or fragmented polypeptides, and/or treating the sample with an enzyme or a chemical agent. Once the sample is provided in the sample container, e.g. in a cartridge, the process 200 moves to prime or flush the system and fluidic connections in 202, by filling the lines with a buffer for example. In some embodiments, one or more lines of the apparatus can be flushed with a gas to clear the lines and/or to remove reagents from the line. In some examples, the one or more lines is flushed with air, argon, or nitrogen. In some aspects, the apparatus is connected to a source for the inert gas. One or more steps of priming the supply line of the apparatus may also be performed, such as by priming the supply line with a reagent. The system then proceeds to 203 to set the temperature of the temperature controlled unit 104 containing the cartridge(s) and deliver a wash solution to the sample in state 204. A loop is performed comprising processes 205-207 repeated n number of times followed by a process 208. During any steps prior to 209 which requires removal of reagents or a wash, the sample container can be evacuated such that solution is removed while the sample containing the macromolecules (e.g. joined to a solid support) is retained in the sample container. The sample can be removed from the sample container using any appropriate means at 209. In some embodiments, prior to or after removal of the sample from the instrument, the sample is prepared for sequencing and analysis. In some embodiments, an amplification reaction may be performed using the apparatus prior to removing the sample from the sample container. A collection means for the sample treated using the apparatus may be further incorporated into the design of the apparatus. For example, the collection means may comprise a connection and a container for collecting the sample or a portion thereof. The collection of the sample or portion thereof may be performed after completing the extension of the recording tag and before analysis of the extended recording tag as described herein. In some cases, the apparatus is configured to allow a collection container to be connected, directly or indirectly, to at least one of the sample container(s).

In some embodiments, the times, temperatures, and/or other conditions, necessary to carry out the reactions performed by the apparatus may be optimized by varying the reaction solutions, temperatures, and/or applying external forces. In some embodiments, the apparatus includes a mixing means or structure. In some cases, the mixing means or structure can include control of fluid flow, e.g. by controlling the movement of an amount of liquid forward and backward through the cartridge. In some embodiments, the mixing means or structure can include control of bubbling air or inert gas through liquid in the sample container. In some embodiments, additional components are added to the apparatus. For example, a mixing means, such as vibration, can be used. In some cases, the apparatus may be designed with closed system architecture which may reduce, minimize or eliminate contamination and difficulties caused by evaporation.

In some embodiments, the apparatus is configured for preserving the reagents. For example, any of the reagent reservoirs may be composed of a material that preserves the reagents e.g., by protecting the reagent contained therein from light, moisture, and/or oxygen exposure. In some embodiments, the tubing or other components of the apparatus may also be composed of a material that preserves the reagents e.g., by protecting the reagent contained therein from light, moisture, and/or oxygen exposure. In some embodiments, the apparatus and/or reagent reservoir is configured to provide an environment suitable for the reagent(s), such as by maintaining an atmosphere of dry inert gas (e.g. nitrogen or argon) above or covering the reagent in its container. In some embodiments, the tubing or other components of the apparatus uses a material that exhibits low-binding for proteins. In some cases, it may be desirable that the material of the tubing or other components of the apparatus is inert to chemicals (e.g. any chemical treatments described herein).

Also provided herein are exemplary uses and applications for using the provided apparatus and automated methods. In some cases, instructions may be provided with the apparatus for operation of the apparatus.

A. Reagent Reservoirs

The provided apparatus comprises one or more reagent reservoirs for containing a respective reagent or a holder or space configured for holding said reagent reservoir(s). In some embodiments, at least one of said reagent reservoirs is subjected to temperature control. In some embodiments, the holder or space configured for holding the reagent reservoir(s) is a temperature controlled unit. In some embodiments, the apparatus includes reagent reservoir(s) for containing any reagents useful for a macromolecule analysis assay (e.g. a polypeptide analysis assay). For example, the reagent reservoirs may contain any or all of the following: buffers, wash buffers, polypeptides, nucleic acids, binding agents, enzymes, chemical reagents for modifying an amino acid, chemical reagents for cleaving one or more amino acids, enzymatic reagents for cleaving one or more amino acids, reagents for a ligation reaction, reagents for a polymerase-mediated reaction, or any combinations thereof. In some embodiments, the reagent reservoirs may contain any of the reagents described for use in the methods provided in Section II. In some embodiments, instructions for performing the method (any steps described in Section II) using the apparatus can be provided in the form of a manual accompanying the apparatus.

In some examples, the apparatus includes at least 1, 2, 3, 4, 5, 6, 7, 8, 9, 10, 20, 30, 40, 50 or more reagent reservoirs. In some embodiments, the apparatus comprises at least one or more reagent reservoir(s) with a volume ranging from about 5 μL to about 50 μL or a holder or space configured for holding said reagent reservoir(s) with a volume ranging from about 5 μL to about 50 μL. In some embodiments, the apparatus comprises at least one or more reagent reservoir(s) with a volume ranging from about 50 μL to about 200 μL or a holder or space configured for holding said reagent reservoir(s) with a volume ranging from about 50 μL to about 200 μL. In some embodiments, the apparatus comprises at least one or more reagent reservoir(s) with a volume ranging from about 200 μL to about 1 mL or a holder or space configured for holding said reagent reservoir(s) with a volume ranging from about 200 μL to about 1 mL. In some embodiments, the apparatus comprises at least one or more reagent reservoir(s) with a volume ranging from about 1 mL to about 50 mL or a holder or space configured for holding said reagent reservoir(s) with a volume ranging from about 1 mL to about 50 mL. In some embodiments, the apparatus comprises at least one or more reagent reservoir(s) with a volume ranging from about 50 mL to about 500 mL or a holder or space configured for holding said reagent reservoir(s) with a volume ranging from about 50 mL to about 500 mL. In some embodiments, the apparatus comprises at least one or more reagent reservoir(s) with a volume ranging from about 500 mL to about 1 L or a holder or space configured for holding said reagent reservoir(s) with a volume ranging from about 500 mL to about 1 L. In some embodiments, the apparatus comprises at least one or more reagent reservoir(s) with a volume ranging from about 1 L to about 100 L or a holder or space configured for holding said reagent reservoir(s) with a volume ranging from about 1 L to about 100 L. In some embodiments, a plurality of reagent reservoirs with a volume of greater than about 50 mL is used to store a bulk reagents such as a wash buffer. In some embodiments, a plurality of reagent reservoirs with a volume of greater than about 1 L is used to store a bulk reagents such as a wash buffer. For example, the apparatus includes at least 1, 2, 3, 4, 5, 6, 7, 8, 9, 10, 20 or more reagent reservoirs with a volume of greater than about 50 mL. In other examples, the apparatus includes at least 1, 2, 3, 4, 5, 6, 7, 8, 9, 10, 20 or more reagent reservoirs with a volume of greater than about 100 mL. In some embodiments, a plurality of reagent reservoirs with a volume of less than about 100 mL is used to store a small volume reagent such as an enzyme or binder mix. For example, the apparatus can include at least 1, 2, 3, 4, 5, 6, 7, 8, 9, 10, 20 or more reagent reservoirs with a volume of less than about 100 mL. In other examples, the apparatus can include at least 1, 2, 3, 4, 5, 6, 7, 8, 9, 10, 20 or more reagent reservoirs with a volume of less than about 50 mL. In some particular examples, the apparatus includes at least 1, 2, 3, 4, 5, 6, 7, 8, 9, 10, 20 or more reagent reservoirs with a volume of less than about 5 mL. In some cases, the placement of the reagent reservoirs may be configured such that reagent reservoirs with a smaller volume are located closer to the sample container than reagent reservoirs with a larger volume.

The reagents may be provided in vials (such as sealed vials), vessels, ampules, bottles, jars, flexible packaging (e.g., sealed Mylar or plastic bags), and the like. In some embodiments, the reagents may be provided in a reusable container, a disposable container, or a recyclable container. In some cases, reagents may be provided in a sterilized and/or sealed format. In some embodiments, the reagent reservoirs may be composed of a material that preserves the reagents e.g., by protecting the reagent contained therein from light, moisture, and/or oxygen exposure. In some embodiments, the reagent reservoir is configured to provide an environment suitable for the reagent(s), such as by maintaining an atmosphere of dry inert gas (e.g. nitrogen or argon) above or covering the reagent in the reagent reservoir.

In some aspects, the reagents may be provided in a lyophilized or other stable or inert form. For example, a reagent provided in a lyophilized or other stable or inert form may be solubilized or resuspended in a solvent (e.g., a buffer) prior to use. In some cases, the apparatus may be used to prepare a reagent for use, such as by mixing the reagent with other components or with other reagents. For example, the apparatus is configured with a pre-mixing chamber for combining two or more reagents in a defined ratio determined by a control program. In some cases, one or more reagent reservoirs contain a subcomponent of a reagent that becomes active when combined with another subcomponent of the reagent. This may be suitable for reagents that might decompose but can be stored as two inert subcomponents. In some instances, the mixed reagent(s) is then delivered to the sample container. In some cases, the apparatus or control program may be configured to adjust the composition of the reagents (or mixtures thereof). In some embodiment, the reagents are provided as subcomponents and mixed or combined by the apparatus to reduce the need for extra reservoirs or allow specialized conditions that would otherwise require manual intervention.

In some embodiments, the reagents are provided in a format that is configured to be used with or compatible with reagent reservoir(s) integrated in the apparatus, or compatible with the holder or space configured for holding the reagent reservoir(s). In some embodiments, one or more reagents is provided in a pierceable package. Each reagent reservoir may be accessible via a port or opening which connects the reagent reservoir containing the reagent to other components of the apparatus.

In some embodiments, the apparatus comprises at least one reagent reservoir comprising a binding agent, or a holder or space configured for holding the reagent reservoir containing binding agent(s). In some cases, the container is suitable for containing a mixture of binding agents, including any appropriate buffers.

In some embodiments, the apparatus comprises at least one reagent reservoir comprising reagents for transferring information, or a holder or space configured for holding the reagent reservoir containing reagents for transferring information. For example, the container is suitable for containing an enzymatic mixture for performing a ligation reaction or an extension reaction, including any appropriate buffers. In addition, the container may also include a mixture of dNTPs. In some embodiments, the apparatus comprises at least one reagent reservoir comprising reagents for transferring information that subjected to temperature control. In some cases, the holder or space configured for holding the reagent reservoir containing reagents for transferring information is temperature controlled. An exemplary mix of Tris-HCl, MgSO4, NaCl, DTT, Tween 20, BSA, dNTPs, and a polymerase (or any combination of the components thereof) can be included as the reagents for transferring information from a coding tag to a recording tag.

In some embodiments, the apparatus comprises at least one reagent reservoir comprising reagents for modifying one or more amino acid(s) of a polypeptide, or a holder or space configured for holding the reagent reservoir for containing the modifying reagent. For example, the reagent for modifying one or more amino acid(s) is a chemical reagent. In some cases, the reagent is for modifying a terminal amino acid, e.g., an N-terminal amino acid or a C-terminal amino acid. In some embodiments, the apparatus comprises at least one reagent reservoir comprising reagents for removing, cleaving, or eliminating one or more amino acid(s) of a polypeptide, or a holder or space configured for holding the reagent reservoir containing the reagent for removing an amino acid. In some cases, the reagent for removing one or more amino acid(s) is a chemical reagent. In some cases, the reagent for removing one or more amino acid(s) is an enzymatic reagent. In some embodiments, the apparatus includes both an enzymatic reagent reservoir and a chemical reagent reservoir. In some cases, the reagent is for removing a terminal amino acid, e.g., an N-terminal amino acid or a C-terminal amino acid. In some embodiments, the apparatus comprises at least one reagent reservoir comprising reagents for removing an amino acid that subjected to temperature control. In some cases, the holder or space configured for holding the reagent reservoir containing reagents for removing an amino acid is temperature controlled. Further chemical or enzymatic reagents for modifying and removing an amino acid are described in Section II.C.2.

In some embodiments, the apparatus comprises at least one reagent reservoir comprising reagents for a capping reaction or a holder or space configured for holding the reagent reservoir comprising reagents for a capping reaction. For example, the container is suitable for containing an enzymatic mixture for performing a ligation reaction or an extension reaction, including any appropriate buffers, for performing a capping reaction. In addition, the container may also include a mixture of dNTPs. In some embodiments, the apparatus comprises a reagent reservoir or a space or holder configured for containing the capping reagent(s) that is temperature controlled. An exemplary mix of a template oligo for a universal priming sequence, Tris-HCl, MgSO4, NaCl, DTT, Tween 20, BSA, dNTPs, and a polymerase (or any combination of the components thereof) may be used as the reagents for the capping reaction.

In some embodiments, the apparatus includes at least two reagent reservoirs containing different types of reagents. For example, each of the reagent reservoirs contains a reagent selected from the group consisting of a binding agent, reagents for transferring information, reagents for removing a terminal amino acid of a polypeptide and reagents for a capping reaction, or holders or spaces configured for holding the reagent reservoirs. In some embodiments, the apparatus includes at least three reagent reservoirs containing different types of reagents. For example, each of the reagent reservoirs comprising a reagent selected from the group consisting of a binding agent, reagents for transferring information, reagents for removing a terminal amino acid of a polypeptide and reagents for a capping reaction, or holders or spaces configured for holding the reagent reservoirs. For example, the apparatus includes at least 1, 2, 3, 4, 5, 6, 7, 8, 9, 10, 11, 12, 13, 14, 15, 20, 25, 30, 40, 50 or 100 reagent reservoirs. In some particular embodiments, the apparatus is configured to hold at least 5 reagent reservoirs. In some particular embodiments, the apparatus is configured to hold at least 10 reagent reservoirs. In some particular embodiments, the apparatus is configured to hold at least 20 reagent reservoirs.

In some embodiments, the apparatus includes at least one reagent reservoir comprising a binding agent, at least one reagent reservoir comprising reagents for transferring information, at least one reagent reservoir comprising reagents for removing a terminal amino acid of a polypeptide, and at least one reservoir comprising reagents for a capping reaction, or holders or spaces configured for holding the reagent reservoirs.

In some embodiments, at least one of the reagent reservoirs of the apparatus contains a binding agent, reagents for transferring information, reagents for removing a terminal amino acid of a polypeptide, and reagents for a capping reaction, or a holder or space configured for holding the reagent reservoir, is subjected to temperature control. In some embodiments, at least two or three of the reagent reservoirs comprising a binding agent, reagents for transferring information, reagents for removing a terminal amino acid of a polypeptide, and reagents for a capping reaction, or holders or spaces configured for holding the reagent reservoirs, are subjected to temperature control. In some particular embodiments, the reagent reservoir comprising a binding agent, the reagent reservoir comprising reagents for transferring information, the reservoir comprising reagents for removing a terminal amino acid of a polypeptide, and the reservoir comprising reagents for a capping reaction, or holders or spaces configured for holding the reagent reservoirs, are subjected to temperature control. In some embodiments, the temperature control for the reagent reservoir is suitable for maintaining a low temperature in order to maintain effectiveness of the reagent. For example, the temperature control for the reagent reservoir is suitable for maintaining a temperature below about 25° C., below about 20° C., below about 15° C., below about 10° C., or below about 5° C. In some examples, accordingly, the reagent container is subjected to cooling. In some examples, the temperature of the reagent reservoir is maintained above 0° C. or the freezing point of the reagent.

In some embodiments, the apparatus includes one or more reservoir containing a wash solution or buffer. In some embodiments, the apparatus includes at least two reservoirs each containing a wash solution. In some embodiments, the apparatus includes at least three reservoirs each containing a wash solution. In some embodiments, the apparatus includes at least 1, 2, 3, 4, 5, 6, 7, 8, 9, 10 or more reservoirs each containing a wash solution. For example, the wash buffer or solution can be selected from PBS (4 mM sodium phosphate, 155 mM sodium chloride), PBST (4 mM sodium phosphate, 155 mM sodium chloride (NaCl), PBF10, (10% formamide, 4 mM sodium phosphate, 500 mM sodium chloride, and 0.1% Tween 20), sodium hydroxide, or any variations thereof. In some embodiments, the wash solution contains formamide, sodium phosphate, sodium chloride, Tween 20, and/or other suitable ingredients. In some embodiments, the apparatus includes a single reagent reservoir that comprises a wash buffer. In some embodiments, the reagent reservoir containing a wash buffer has a volume of about 5 mL to about 50 mL, about 10 mL to about 100 mL, about 50 mL to 500 mL, or about 100 mL to about 1 L. In some cases, the reagent reservoir comprising the wash buffer is configured to hold a volume of about 50 mL or more. In some embodiments, the apparatus includes multiple reagent reservoirs each containing different wash buffers, e.g., three or more different wash buffers.

B. Sample Container

The provided apparatus comprises one or more sample container(s), wherein at least one of said sample container(s) is subjected to temperature control and configured for allowing fluid flow-through. In some cases, the apparatus includes n number of sample containers 105 contained in a temperature controlled unit 104 as shown in FIG. 1A-1C. In some embodiments, the one or more sample containers are held in a space that is temperature controlled. In some cases, the apparatus includes a holder or space configured for holding said sample container(s). In some embodiments, at least one of the sample container(s) is configured to be loaded or provided with a starting sample liquid. In some embodiments, each sample containers is loaded with a sample by the apparatus from an input reservoir. For example, a sample can be loaded onto the apparatus and the apparatus delivers the sample from the input reservoir to the sample container. In some embodiments, the sample container(s) are connected to the one or more input reservoirs via a supply line, wherein the supply line is optionally a common line. In some other cases, the cartridges may be removable from the apparatus. For example, the sample container(s) can be loaded by the user with a sample containing a macromolecule, e.g., a polypeptide.

Suitable non-planar sample containers may be made of various materials and shapes. In some embodiments, the sample container is compatible for use with a support which comprises a three-dimensional material (e.g., a gel matrix or a bead). The sample container can be loaded with a sample that contains macromolecules immobilized on a support. In some embodiments, it is preferred to immobilize the macromolecules from the sample using a three-dimensional support (e.g., a porous matrix, a bead, a substrate comprising micro-fabricated pillar structures, or a microfluidic substrate with microfabricated structures) (see e.g., US 2016/0001199 A1). In some cases, desirable properties for the sample container includes low-binding for proteins. In some cases, it is desirable that the material of the sample container is inert to chemicals (e.g. any chemical treatments described herein). In some particular embodiments, the sample container is made of a material that is compatible with or transparent to microwave application. For example, the sample container can be made of a material that comprises glass, a glass-like material (e.g., fused silica, quartz), polyether ether ketone (PEEK), and polytetrafluorethylene (PTFE), fluorinated hydrocarbon plastics, or any combination thereof. As described herein, the non-planar sample container can comprise a top and a base, and side walls connecting the top and the base.

In some embodiments, the sample container is configured to use in the apparatus such that the delivery of liquids (e.g., reagents) is via discrete and non-continuous flow. In some cases, this discrete and non-continuous flow is advantages for exchange of liquids applied to the sample container and removal of reagents from the sample container. For example, a first reagent may be delivered to the sample container, and after incubation, the first reagent can be nearly completely evacuated from the sample container before a second reagent is delivered to the sample container, thereby reducing the amount of mixing between the first and second reagents. This discrete delivery and removal of reagents to and from the sample cartridge may create an air gap in the sample container. In some embodiments, the sample container has a vent or valve. For example, the sample container has a valved opening. In some cases, the sample container may comprise a valved opening to atmospheric pressure. The vent or valve may be useful in some cases to release pressure displaced by liquid entering the sample container. In some specific embodiments, the sample container has a vent or valve that opens to atmospheric pressure so that a reagent can be pulled out of cartridge and replaced by air, prior to delivery of the next reagent or wash buffer to the sample container. In other embodiments, the flow of liquid into the sample container is continuous. The sample container may be subjected to positive pressure, such as applied by a pump.

In some embodiments, the sample container and apparatus use a system design where a gas is delivered via the reagent supply line and pushed through to a waste container. For example, the sample container is not vented or the vent is closed and the gas is delivered to the sample container and evacuated through an outlet to a waste container. In some embodiments, flushing the supply line with a gas and/or delivering a gas to the sample container may be desirable to substantially or fully remove or flush any leftover buffers and/or reagents.

In some embodiments, the sample contained is a sealed cartridge. One advantage of a sealed cartridge and/or system is the prevention of leaks. The sample container, in some cases, can be under negative pressure. For example, a pump can be positioned downstream of the sample container to apply negative pressure to the sample cartridge. Some benefits with a sample cartridge that is subjected to negative pressure may include improved flow characteristics, especially with a reaction volume that is about 50 μL to about 100 μL. In some aspects, other desired features might be a sample contained that is easier, fast, better controlled, and/or more efficient to deliver reagents to and/or drain.

While the top and the base of the sample container are described in its upright position (vertically), the sample container may also be placed on its side in relation to the apparatus (horizontally). The non-planar sample container may be characterized as having a significant height to the container that is not essentially flat. In some embodiments, a planar sample container is characterized by: a) having at least one dimension (e.g., length, width, or diameter) that is greater than its height; b) having a ratio between the height and largest dimension (e.g., length, width, or diameter) from about 1:2 to about 1:10, from about 1:2 to about 1:50, from about 1:2 to about 1:100, or from about 1:2 to about 1:500; and/or c) having a thickness or height of equal to or less than 1 mm. In some embodiments, the non-planar sample container configured for use with the provided apparatus is characterized by: a) having at least one dimension (e.g., length, width, or diameter) that is less than its height; b) having a ratio between the height and largest dimension (e.g., length, width, or diameter) from about 1:1 to about 10:1, from about 1:1 to about 20:1, from about 1:1 to about 50:1, or from about 1:1 to about 100:1; and/or c) having a thickness or height of greater than 1 mm. The provided apparatus is configured for use with a sample container which is not a planar container. A planar container may have minimal height (e.g., depth or thickness) between the top and bottom of the container to allow continuous laminar flow.

In some embodiments, the top and the bottom of the sample container comprise an inlet and an outlet for the delivery of reagents. In some aspects, the inlet of the container is also used for the initial delivery of the sample(s) to the sample cartridge(s). In some embodiments, the sample container is or comprises a cartridge. In some embodiments, each sample container is a removable and replaceable component of the apparatus. In some embodiments, the sample container is not a patterned flow cell for sequencing a nucleic acid sample. In some embodiments, the sample container is not a slide on which a planar sample is deposited.

In some embodiments, the apparatus is configured to hold a single sample container, or to hold two or more sample containers. The temperature controlled unit for the sample container(s) may be of any shape so long as the unit can hold sample containers (e.g., cartridges) while providing certain functions and advantages of the present disclosure. In some embodiments, the temperature controlled unit is configured to hold a single sample container, or to hold two or more sample containers. In some embodiment, the apparatus is configured to hold at least 1, 2, 3, 4, 5, 6, 7, 8, 9, 10, 20, 30, 40, 50, or 100 sample containers. In some embodiment, the apparatus is configured to hold 2 to 10 sample containers. The apparatus may be designed such that not all sample container slots on the apparatus capable of holding a sample container has to be loaded and used at all times (e.g. some may be inactive). In some embodiments, each sample container has a volume (e.g., capacity of the container) equal to or less than about 50 mL, equal to or less than about 20 mL, equal to or less than about 10 mL, equal to or less than about 5 mL, equal to or less than about 2 mL equal to or less than about 1 mL, equal to or less than about 0.5 mL, or equal to or less than about 0.25 mL. In some specific embodiments, each sample container has a volume of equal to or less than about 20 mL. In some specific embodiments, each sample container has a volume of equal to or less than about 10 mL. In some specific embodiments, each sample container has a volume of equal to or less than about 1 mL.

In some embodiments, at least one of the sample container(s) and/or at least one of the reagent reservoirs is subjected to active heating. In some embodiments, at least one of the sample container(s) and/or at least one of the reagent reservoirs is subjected to active cooling. Any suitable means for applying temperature control may be used. For example, it may be desired for the sample container to be cooled or heated in a relatively and sufficiently fast manner for efficiently performing the reactions for treating the sample. In some examples, the temperature control of the sample container uses air, chilled air, a surface in contact with the sample container, or liquid cooling. In some cases, thermoelectric cooling or heating is used to moderate or modulate temperature of the sample. For example, a Peltier cooler or heater can be used to moderate or modulate temperature of the sample. In some embodiments, the provided apparatus includes a means or structure for monitoring the temperature of one or more of the sample container and providing feedback control of the temperature. In some embodiments, the apparatus includes a separate sensor and temperature control for each sample container (for each cartridge) or for each thermal block. In some aspects, pressure within the sample container is monitored.

In some embodiments, the apparatus includes multiple sample containers, wherein at least one of the sample containers is subjected to temperature control and configured for allowing fluid flow-through, or a holder or space configured for holding the sample containers. In some embodiments, the apparatus includes multiple sample containers that are subjected to temperature control and configured for allowing fluid flow-through, or a holder or space configured for holding the multiple sample containers. The apparatus may include one or more individually controlled and modulated temperature blocks.

In some embodiments, at least one of the sample container(s) comprises a porous means or a porous membrane to allow a liquid to pass through and evacuate the sample container and/or to maintain a sample, e.g., a sample liquid, in the sample container. In some cases, the sample container(s) includes a filter means or a frit for retaining the sample while allowing flow-through of other materials (e.g. buffers). In some embodiments, the porous means or porous membrane is for retaining the sample from evacuating through the outlet of the sample container. Meanwhile, reagents and buffers may flow through the sample container and evacuate the sample container through the outlet of the sample container. Any suitable porous material can be used as the filter means. Suitable filter means may include desired characteristics including diameter, pore size, and thickness of the material. In some cases, the filter means comprises a non-reactive material. In some cases, the filter means comprises a material that does not bind to the components of the macromolecule analysis assay. In some embodiments, the filter means is made of a hydrophobic material. In some embodiments, the filter means is made of a material that comprises polyethylene (PE), polytetrafluorethylene (PTFE), or a similar hydrophobic material.

In some embodiments, the filter means is configured and positioned to fit in the cartridge. In some examples, the filter means (e.g., frit) has a pore size from about 1 μm to about 500 μm. In some examples, the frit has a pore size of less than about 50 μm, less than about 40 μm, less than about 30 μm, less than about 20 μm, less than about 10 μm, less than about 5 μm, less than about 4 μm, less than about 3 μm, less than about 2 μm, or less than about 1 μm. In some specific examples, the filter means (e.g., frit) has a pore size from about 1 μm to about 5 μm. The filter means (e.g., frit) can be of any suitable thickness and can be adjusted based on various factors including the material used and the filtering effects desired. In some examples, the frit has a thickness of about 0.1 mm to about 5 mm, about 0.1 mm to about 1 mm, about 0.1 mm to about 0.5 mm, about 0.2 mm to about 5 mm, about 0.2 mm to about 1 mm, about 0.2 mm to about 0.5 mm. In some instances, the frit has a thickness of about 0.5 mm. In some embodiments, the sample container contains, or is loaded or prepared with support(s). For example, the sample container may be loaded with support(s) (e.g. beads) that are configured for capturing macromolecules with associated and/or attached recording tags.

In some examples, each sample container has an inlet for the delivery of reagents and an outlet for evacuation of reagents. In some embodiments, the outlet of the sample container(s) is configured for draining liquid from the sample container(s) to a waste container. In some cases, the waste container is fluidically connected to one or more sample containers, directly or indirectly. In some examples, the apparatus comprises more than one waste container. For example, the apparatus may include a waste container for storing a particular type of waste, e.g., organic waste.

In some embodiments, the sample container(s) are connected to the one or more reagent reservoir(s), see FIG. 1A-1C. In some embodiments, the sample container(s) are connected to the one or more reagent reservoir(s) via a supply line. In some cases, the supply line is a common line. In some embodiments, the movement of the fluid to and from the sample container is controlled using a pump.

In some embodiments, the apparatus further comprises a means for collecting the sample or a portion thereof released from the sample container. In some cases, the means for collecting the sample or a portion thereof comprises a collection container connected, directly or indirectly, to at least one of the sample container(s). In some examples, the sample container(s) is connected via tubing and an additional valve to a collection container. In some embodiments, the sample is treated with a cleaving reagent prior to collection, such that the recording tags are released and collected. The sample collection or recovery can be an automated process. In some embodiments, the collection or recovery process for the sample may include a run-off procedure for collecting the sample, eluding the sample, controlling any valves involved in the exit of the sample, and directing the sample for collection in a collection container or receptacle.

C. Control Unit and Process

In some embodiments, one or more aspects of the apparatus function is controlled by a control system or unit. The control unit may be used to automate one or more processes performed using the apparatus. In some examples, a control unit 108 is used to carry out one or more steps of process such as depicted in FIG. 2A-2C. In some aspects, the control unit is used to automate and/or control the temperature of the sample container(s). In some embodiments, the control unit is used to automate and/or control the temperature of the reagent reservoir(s). In some aspects, the control unit is used to automate and/or control the flow of liquids in the apparatus. In some aspects, the control unit is used to automate and/or control the positioning of the valve(s). In some cases, the control unit is used to automate and/or control and/or delivery of said one or more reagent(s) to said sample container(s).

A computer with associated electronics and software controls numerous aspects of the process including the opening and closing of valves for the desired time period, the sequence of altering positions of the valves, the movement of the pump, the proper incubation period for each reagent addition to the sample or sample container, and the evacuation of the content in the sample container after the incubation period is complete. In some aspects, the control unit is used to automate and/or control the temperature of the sample container(s). In some embodiments, the control unit is used to automate and/or control the temperature of the reagent reservoir(s). In some aspects, the control unit is used to automate and/or control the flow of liquids in the apparatus. In some aspects, the control unit is used to automate and/or control the positioning of the valve(s). In some cases, the control unit is used to automate and/or control and/or delivery of said one or more reagent(s) to said sample container(s). For example, FIG. 1A-1C depicts a control unit 108, which can be used to carry out one or more steps of process such as depicted in FIG. 2A-2C. In some embodiments, the temperature control of the sample container(s) is automated and/or controlled by the control unit. In some embodiments, the temperature control of the reagent reservoir(s) is automated and/or controlled by the control unit. In some embodiments, the positioning of the valves is automated and/or controlled by the control unit. In some embodiments, the delivery of one or more reagents to the sample container is automated and/or controlled by the control unit. In some embodiments, the time for a reaction and/or cycles of reactions is automated and/or controlled by the control unit.

In some embodiments, the control system or unit is programmable by the user. Any of the steps of the protocol or control program can be optimized. In some embodiments, the apparatus includes a graphical user interface. In some examples, the control unit is programmed by the user to determine the sequence and rate in which the fluid flows from the reagent reservoir(s) to the sample container. The user may adjust the control program as necessary based on the reagents delivered and processes performed. For example, viscosity of a particular reagent or sample may require slower flow rates. In some examples, the control unit is programmed by the user to determine the temperature of sample container at each step of the process carried out by the apparatus. In some embodiments, the control unit is in communication with one or more valves to determine the position of the valve. In some cases, the temperature of the sample container(s) and/or the reagent reservoir(s) can also be controlled by providing power to the heaters or coolers (of a temperature control unit/thermal block) for variable periods of time.

In an exemplary system, the diagrams in FIG. 1A-1C depicts a control unit or processor in the apparatus. The control unit 108 may comprise a computer processor operable to control the valves 102 to allow control of the reagents sent through positionable valve through the apparatus. In some aspects, the control unit can be used to control or automate positioning of a means for moving one or more reagent, e.g., any pumps 103. In some embodiments, the pump is a syringe pump or other pumping devices (e.g., vacuum pump, micropump, etc.) that can generate a pressure differential, which further comprises a means for moving the one or more reagent, e.g., the one or more reagent liquid. In some cases, a means or structure for applying or delivering gas pressure is controlled by the control unit. In some embodiments, the apparatus includes a single pump. In some embodiments, the apparatus includes a plurality of pumps. In some examples, the one or more pump(s) is integrated into the apparatus. In some embodiments, the pump is external of the apparatus. In some embodiments, positive and/or negative pressure can be applied to the sample container(s). In some cases, a negative pressure means (e.g. vacuum) may be applied to remove the reagents (wash buffers) from the sample container(s) to the waste reservoir. The apparatus may include at least two pumps, including for example, a syringe pump for delivery of reagents and a vacuum pump for evacuation of reagents from the sample container. In some cases, the apparatus may be configured such that if a pump is needed to be cleared between deliveries of reagents to the sample container, a bypass can be included such that during an incubation step in the sample container, the bypass allows the pump to be cleared during the incubation. In some embodiments, one or more of the pumps comprises a micropump. In some cases, the number of pumps needed can be adjusted based on the need of the sample container and the number of sample containers to be processed. For example, an apparatus can be designed to support a 96-well plate format sample container.

Any suitable programmable language may be used for the control program. In some cases, control unit is configured to be operated using a cross-platform language. In some examples, the computer program or software may include any sequence or human or machine cognizable steps which performs a function. Such program may be rendered in virtually any programming language or environment including, for example, C/C++, C #, Fortran, COBOL, MATLAB™, PASCAL, Python, assembly language, markup languages (e.g., HTML, SGML, XML, VoXML), Java™ (including J2ME, Java Beans, etc.), Binary Runtime Environment (e.g., BREW), scripting languages (e.g., Sh, Bash, Perl), and any variants thereof. In some specific cases, the control unit is operated using Python. In some embodiments, the apparatus may include a control unit that is programmable or can be modified such that the system allows the user to create, change, and adjust numerous system settings, running parameters for various processes to suit various needs. In some embodiments, the apparatus may include a control unit that provides suitable parameters and settings for various processes such that automation is provided and little user input is needed.

In some embodiments, the apparatus is compatible with barcode technology such that reagents and/or samples can be associated with a barcode. In some cases, the barcode can be used to track any suitable and useful information for the samples and/or the processes. In some cases, examples of information content of the barcode may include names of the reagents, manufacture information such as date and expiration, any serial numbers, reagent volumes, sample types, protocol information, etc. In some embodiments, the apparatus includes a detector for a machine-readable signal, e.g., a barcode reader or radio-frequency identification (RFID) reader. In some further embodiments, the control unit or processor may comprise a database or access to a database for processing information regarding the sample.

In some embodiments, the control unit can be used to automate and/or control delivery of said one or more reagent(s) to said sample container(s). In some aspects, the delivery of one or more reagents is individually addressable, e.g., for each sample container. In some cases, the control unit carries out delivery of a single reagent to a single sample container or to multiple sample containers. In some cases, the control unit controls delivery of multiple reagents to multiple sample containers.

In some embodiments, the control unit can be used to automate and/or control the position of valve(s). In FIG. 1A, an exemplary system is depicted where all reagent valves and sample container (e.g. cartridge) valves are closed and the pump delivers bypass to the waste container. In FIG. 1B, an exemplary system is depicted where the pump aspirates one reagent. In FIG. 1C, an exemplary system is depicted where the pump delivers a reagent from the reagent-containing reservoir to the sample container (e.g. cartridge). As a first step prior to treating any samples in the sample container, the appropriate valves may be opened in order to prime the supply line with a desired reagent from a reagent reservoir. The valves of the apparatus may operate in different configurations (e.g., open or closed) to either release fluid into a path, remove fluid from a path, or prevent fluid from entering a path. In some embodiments, the apparatus comprises two or more valves. In some cases, two or more of the valves are integrated in a manifold. The valves may be selected based on desired characteristics, such as a small dead and/or swept volume. For example, the apparatus can comprise microvalves with dead volumes of about 0.5-5 μL, about 1-10 μL, about 1-5 μL, about 1-4 μL, about 1-3 μL, or about 1-2 μL. For example, the apparatus can comprise microvalves with swept volumes of about 1-10 μL, about 1-20 μL, about 1-50 μL, about 10-20 μL, about 10-50 μL, or about 20-50 μL. In some cases, the valves are selected from rotary valves, solenoid valve selection valve, slider valve, diaphragm valve, pinch valve or other suitable valves. In some embodiments, one or more valves (e.g. a 4-way manifold) can be used to control flow from the sample container (e.g. exit or drain for each sample container).

In some embodiments, the control unit is used to automate and/or control the temperature of the sample container(s). For example, the preparation and treatment of the macromolecules in the sample container may include cycling between various temperatures for each desired reaction. The control unit may be used to automate exemplary temperature changes between about 4° C. (+/−1° C.), 8° C. (+/−1° C.), 25° C. (+/−1° C.), 30° C. (+/−1° C.), 40° C. (+/−1° C.), 60° C. (+/−1° C.), 80° C. (+/−1° C.), in any order or combinations. The user may adjust the temperature settings for a reaction based on a number of factors for each reaction including incubation with binding agents, transferring information to a recording tag, modifying an amino acid, and removing at least one terminal amino acid (via a chemical or enzymatic treatment).

In some embodiments, the control unit receives feedback from one or more components of the apparatus. In some embodiments, the control system receives feedback from one or more valves, the temperature controlled unit, and/or one or more sample containers. In some embodiments, the feedback from monitoring the apparatus provides information regarding reagent delivery. For example, the feedback can include information from monitoring temperature, pressure, flow, air bubble, position of one or more of the valves, refractive index, and/or conductance. In some embodiments, the apparatus is configured to provide feedback of the monitoring to the control program. In one example, the opening or closing of valves or changes in potential is controlled by the processor, which is further in communication with one or more detectors which monitors the components in different paths within the upstream separation module. In some aspects, feedback regarding the position of a valve is provided as feedback to the control unit. The feedback from the valve(s) can be binary or have positional information and can be dependent on the component (e.g., type of valve used).

In some embodiments, the apparatus includes a means for detecting failed deliveries of reagents to the sample container(s). If failed delivery of a reagent is detected, the control system can pause or stop the running process, and optionally take any suitable further actions to repeat the delivery of the reagent. The delivery of reagents can be monitored in any suitable manner, including using a bubble sensor, such as a photoelectric device. In some cases, the monitoring is performed outside of the sample container, and does not disrupt the fluid stream to the sample container. In some embodiments, the control unit or program provided can specify that the expected delivery of a reagent is a specified amount. The monitoring can resolve the amount of the volume that was aspirated and delivered and set an amount of deviation that is permitted by the system and an amount of deviation that is unacceptable and considered a failed delivery event. In some cases, the resolution of the monitoring of reagent delivery can be sub-microliter. In one example, if the delivery of the reagent monitored is less than 50% of the volume that was requested, then the control unit considers the reagent delivery event as a failure then can take a recovery action, including moving the failed delivery to waste, repeating the reagent delivery, pausing or putting the run in a safe state, and/or specify how many times to tolerate a failed delivery before ending the run. In some cases, a mass flow sensor can measure volumes of expected reagent gain or loss in any particular steps.

In some embodiments, the evacuation of the sample container is monitored and/or feedback is provided from sensors configured to provide information regarding the evacuation of the sample container. For example, a pressure sensor and/or a mass flow sensor, can be used to detect the vacuum used to evacuate reagents from the sample container, detect the timing for evacuation, and sufficient pressure for evacuation. In some cases, if low or lack of volume is detected, the pump can be directed by the control unit to adjust pressure to compensate, or a different pump can be employed to compensate. In some embodiments, system performance over time is monitored to detect any decline in function. For example, if any decline in performance is detected in a pump, a regulator (such as a vacuum regulator) could be applied to the apparatus.

In some embodiments, the apparatus includes an analytical means to monitor function and performance of the apparatus. This monitoring and feedback may be used to stop a process if an error occurs in the function of any of the processes carried out by the apparatus and a correction can be made. In some embodiments, the apparatus includes an illumination means. In some cases, the apparatus include a means or a sensor for detecting a detectable signal, e.g., a fluorescent signal. For example, the sample may be processed in one or more steps to include an indicator (e.g., a fluorescent indicator) that a particular reaction has occurred. In some aspects, the detectable signal is a quality control indicator generated by the sample collectively. In some cases, the detectable signal is indicative of a characteristic of the sample collectively. In some cases, the detectable signal is not indicative of the sequence of an individual macromolecule. In some embodiments, the apparatus comprises a yield detector. In some embodiments, a fluorescence readout may be indicative of yield, such as yield from amplifying the extended recording tags.

D. Optional Microwave Generator

In some embodiments, the provided apparatus and methods for treating a sample may include the application of radiation, e.g., electromagnetic radiation or microwave energy (e.g., radio frequency, RF). In some embodiments, the described chemical and physical processes may be performed within a microwave radiation field, as depicted in FIG. 1D. In some embodiments, one or more steps of the processes can be accelerated by applying microwave energy to the sample. For example, microwave energy may be applied to the sample that is contacted with a reagent to functionalize or modify an amino acid of a polypeptide in the sample (e.g., NTAA). In some embodiments, microwave energy may be applied to the sample that is contacted with a binding agent capable of binding to the macromolecules (e.g., polypeptides) in the sample. In some aspects, microwave energy may be applied to the sample that is contacted with a reagent to remove an amino acid (e.g., NTAA) from a polypeptide. In some embodiments, the application of microwave energy is automated and controlled by the control unit.

In some embodiments, the contacting of the polypeptide with a reagent in the sample container (e.g., with a functionalizing or modifying reagent, with a binding agent, or with a reagent to remove one or more amino acid(s)) is performed in a cavity in communication with, exposed to, or connected to a microwave radiation source (e.g., RF source). In some examples, the contacting of the polypeptide with any of the reagents or binding agents provided herein is performed in a microwave chamber (See e.g., U.S. Patent Application Publication Number US 2013/0001221; International Patent Publication No. WO 2012/075570). In some embodiments, the provided methods are performed in a single-mode microwave cavity. In some cases, the provided methods are performed in a multimode microwave cavity.

Equipment and reagents of standard type may be used in the present method. In one embodiment, the method is performed in a sample container wherein the temperature and/or pressure may be monitored and optionally moderated. In some examples, the temperature is monitored using a non-invasive method, e.g., an infrared camera.

In some embodiments, the temperature of the sample within the sample container is monitored. In some embodiments, the pressure of the sample container is vented via a pressure vent in the sample container. In some examples, a control system controls and adjusts the microwave source based on feedback such as power absorbed, temperature, pressure, of the sample. In some embodiments, the temperature is monitored and/or controlled at any or all step(s) of the methods provided herein. For example, the temperature may be adjusted to a suitable value or maintained at a suitable level determined by the skilled person. In some embodiments, the method is performed in a sample container that may have cooling applied. For example, active cooling (e.g., air cooling) may be applied to the sample container. In some embodiments, temperature is controlled within the range of about 10° C. to 200° C., about 10° C. to 150° C., about 10° C. to 100° C., about 20° C. to 200° C., about 20° C. to 150° C., about 20° C. to 125° C., about 20° C. to 100° C., or about 25° C. to 125° C. In some cases, the temperature is moderated (e.g. cooled) such that the sample in the sample container is rapidly cooled. In some examples, the moderation of the temperature is performed using air, chilled air, a surface in contact with the sample container, or liquid cooling. In some cases, thermoelectric cooling or heating is used to moderate or modulate temperature of the sample. For example, a Peltier cooler or heater can be used to moderate or modulate temperature of the sample.

In some embodiments, tuning can be applied to the microwave reaction. In some cases, various changes can result in change in the microwave energy needed or applied, including the size, contents (including fluid nature of the sample and/or reagents and any ionic changes), material, or position of the sample container. In some aspects, tuning rods or structures can be included in the microwave cavity to change field intensity of the microwave energy. The tuning mechanism may allow a flexible way to control and modify the application of the field intensity if different reagents are used. To monitor the energy applied to a sample under given conditions, a spectrum analyzer can be used. Various characteristics of the tuning rod can be modified, including the number of rods or other characteristics of the rods (e.g., Keats et al., IFAC Mechatronic Systems (2004) 37(14): 253-258).

In some embodiments of the provided methods, the reactions may also be quenched, such as by reducing the overall reaction temperature. There are a number of parameters that can be controlled and specified with the microwave source or generator. For example, parameters may include time, temperature, pressure, cooling, power, mixing, pre-stirring, initial power, dielectric of solution, vial type or material, and/or absorption. In some embodiments, microwave instruments may provide controllable, reproducible and fast application of energy under conditions where rapid cooling down of the reaction can take place.

In some embodiments, the microwave energy (e.g., radio frequency, RF) is generated by a solid-state microwave power amplifier. In some examples, the power amplifier can vary both the microwave power (e.g., 0-10 W or 0-100 W or 0-1000 W) and frequency (e.g., 2.3-2.7 GHz). In some examples, the microwave energy is applied to a sample in a single mode resonant cavity. For example, the dimensions of the cavity are designed to enable excitation of a single-mode of the cavity to create a single standing wave with the time-averaged electric field (E field) maximal at the sample positioned in the center of the cavity (See e.g., Koyama et al., Journal of Flow Chemistry (2018) 8(3): 147-156; Barham et al., Chem Rec (2019) 19(1): 188-203; Odajima et al. Chem rec (2019 19(1):204-211). In a preferred embodiment, a single-mode microwave irradiation system in which microwave excitation is radiated as a single standing wave, and the time-averaged electric field is maximal at a sample-containing container positioned in the center of the cavity, is used to uniformly heat the volume of the sample.

In some embodiments, the microwave energy generator is in communication with a control unit. In some embodiments, the electric field and/or cavity exposed to the microwave energy is in communication with the microwave energy generator and/or the control unit. In some cases, the control unit and/or microwave generator is in communication with an electric field sensing element and a thermal sensing element. In some embodiments, the power and frequency of the microwave radiation are controlled automatically by feedback from an electric field sensing element and a thermal sensing element (See e.g., Koyama et al., Journal of Flow Chemistry (2018) 8(3): 147-156; Barham et al., Chem Rec (2019) 19(1): 188-203; Odajima et al. Chem rec (2019 19(1):204-211). An autotuning of frequency feature from these feedback elements, can be used to adjust the microwave frequency to stay in tune with the changing resonant modes of cavity/container system (e.g. the resonant frequency of cavity/sample container shifts with changes in solution type, i.e. dielectric/permitivity differences between solutions, in the sample container and with temperature of the sample container).

In some embodiments, the microwave energy has a wavelength from about one meter to about one millimeter, e.g., a wavelength from about 0.3 m to about 3 mm. In some cases, the microwave energy has a frequency from about 300 MHz (1 m) to about 300 GHz (1 mm). In some embodiments, the microwave energy has a frequency from about 1 GHz to about 100 GHz. In some embodiments, the microwave energy has a frequency from about 0.5 GHz to 500 GHz, from about 0.5 GHz to 100 GHz, from about 0.5 GHz to 50 GHz, from about 0.5 GHz to 25 GHz, from about 0.5 GHz to 10 GHz, from about 0.5 GHz to 5 GHz, or from about 0.5 GHz to 2.5 GHz, 2 GHz to 500 GHz, from about 2 GHz to 100 GHz, from about 2 GHz to 50 GHz, from about 2 GHz to 25 GHz, from about 2 GHz to 10 GHz, from about 2 GHz to 5 GHz, or from about 2 GHz to 2.5 GHz. In one example, the microwave generator operates at about 902-928 MHz. In a preferred embodiment, the microwave energy has a frequency from about 2.44 GHz to 2.46 GHz. In one example, the microwave generator operates at 2.45 GHz+−0.2 GHz.

In some embodiments, the microwave energy has a frequency with an IEEE radar band designation of S, C, X, Kᵤ, K or Kₐ band. In some embodiments, the microwave energy has a photon energy (eV) from about 1.24 μeV to about 1.24 meV, e.g., at about 1.24 μeV to about 12.4 μeV, about 12.4 μeV to about 124 μeV, about 124 μeV to about 1.24 meV. In some examples, the microwave energy is applied at about 5 watts, about 10 watts, about 15 watts, about 20 watts, about 25 watts, about 30 watts, about 35 watts, about 40 watts, about 45 watts, about 50 watts, about 60 watts, about 70 watts, about 80 watts, about 90 watts, about 100 watts, about 110 watts, about 120 watts, about 130 watts, about 140 watts, about 150 watts, about 300 watts or higher watts, or a subrange thereof. In some embodiments, the microwave is generated by an amplifier capable of delivering between about 0 W to 10 W, 0 W to 50 W, between about OW to 100 W, between about OW to 200 W, between about OW to 300 W, between about OW to 400 W, between about OW to 500 W, or between about 25 W to 200 W. The microwave energy may be adjusted to a suitable value or level determined by the skilled person based on the characteristics of the sample, for example, volume of the sample.

In some embodiments, the microwave energy is applied for a time period of about 1 minute, 2 minutes, 3 minutes, 4 minutes, 5 minutes, 10 minutes, 15 minutes, 20 minutes, 25 minutes, 30 minutes, 35 minutes, 40 minutes, 45 minutes, 50 minutes, 1 hour, or a loner time period, or a subrange thereof, for any or each of the step(s) of any of the methods provided herein. In some embodiments, the microwave energy is applied to the polypeptides prior to or after any or each of the steps(s) of any of the methods provided herein. In some embodiments, the microwave energy is applied for a duration of time effective to achieve modification of, binding to and/or removal of an amino acid in at least 20%, 30%, 40%, 50%, 60%, 70%, 80%, 90% or greater percentage of the polypeptides.

In some embodiments, the microwave energy is applied by a non-uniform microwave field. In some embodiments, the microwave energy is applied by a uniform microwave field, e.g., applied by microwave volumetric heating (MVH).

In some embodiments, the microwave energy is applied or delivered uniformly to a sample in a sample container. In some cases, the sample container exposed to microwave energy comprises aqueous and/or organic material.

In some embodiments, the microwave energy is applied in the presence of an ionic liquid. For example, the microwave energy is applied to the mixture of the polypeptides in an ionic liquid.

In some embodiments, the methods provided herein are performed to maintain the reaction at a fixed temperature. In some examples, the methods provided herein are performed to maintain the reaction at a temperature of about at least 10° C., 20° C., 30° C., 40° C., 50° C., 60° C., 70° C., 80° C., 90° C., or 100° C., or a subrange thereof. In some cases, the methods provided herein are performed to maintain the reaction at a temperature of about 30° C., 60° C., or 80° C., or a subrange thereof. A solid-state MW generator is used to apply MW energy to a single mode resonant cavity. In a preferred mode, the MW Generator operates at 2.45 GHz+−0.-0.05 GHz. The dimensions of the MW cavity are designed to enable excitation of a single-mode of the cavity to create a single standing wave with the electric field concentrated at the cartridge positioned in the center of the cavity as depicted in FIG. 1D. The dashed curved line in the microwave cavity indicates the time averaged absolute value of the single mode electric field intensity within the MW cavity. The intensity of the E field is maximal at the center of the cavity where the sample cartridge is positioned.

### II. Automated Methods for Performing a Macromolecule Analysis Assay

Provided herein are methods for automated treatment of a sample containing macromolecules (e.g., peptides, polypeptides, and proteins). In some embodiments, one or more steps for treating macromolecules associated with a recording tag in a macromolecule analysis assay are automated. One or more steps of the preparation of the sample for the analysis assay can be performed in an automated manner. For example, the treatment of the macromolecules (e.g., peptides, polypeptides, and proteins) in the sample can be treated with various chemical or enzymatic reagents to prepare the sample, such as by joining the macromolecule to a recording tag. In some cases, the loading of the prepared samples onto the apparatus for the assay can be performed in an automated manner. In some particular embodiments, the macromolecules with associated and/or attached recording tags are immobilized on a support and subjected to a polypeptide analysis assay. In some cases, the macromolecule analysis assay is performed to assess the macromolecule, or to prepare a sample to identify or determine at least a portion of the sequence of the polypeptide macromolecule. In some embodiments, a plurality of macromolecules are prepared for analysis using the described methods to enable downstream analysis of the sequence of single individual peptides, polypeptides, or proteins. The apparatus as described in Section I may be used to perform and automate any of the steps of the provided methods. In some embodiments, the methods provided herein comprise a cyclic process for converting a peptide sequence into DNA encoded information. For example, the polypeptide analysis assay may include repeating steps of binding at least one terminal amino acid of the polypeptide, transferring information from a coding tag to a recording tag, and cleaving at least one terminal amino acid of the polypeptide in a cyclic manner. In some embodiments, the methods include any combinations of the following: enzymatic reaction, an aqueous-phase biochemical reaction, and/or an organic reaction.

In some embodiments, the macromolecule analysis assay is performed to identify, quantify, characterize, distinguish, or a combination thereof, all or a portion of the components of the macromolecule. In some embodiments, the macromolecule analysis assay is performed for analysis of proteins, polypeptides, peptides, nucleic acid molecules, carbohydrates, lipids, macrocycles, chimeric macromolecules, or any combinations thereof. In some embodiments, the macromolecule analysis assay is performed to analyze two or more macromolecules. In some examples, the macromolecule analysis assay includes the binding or contacting of a probe to a macromolecule. In some embodiments, the probe is labeled with an oligonucleotide such as a nucleic acid tag. In some embodiments, the probe comprises a small molecule. In some cases, the macromolecule analysis assay includes a small molecule reactive probe. In some embodiments, the probe interacts with, reacts with, or binds to at least a portion of the macromolecule. In some embodiments, the probe binds to or interacts with the macromolecule at a reactive site. In some embodiments, the probe binds to a binding site of a macromolecule. In some embodiments, the probe binds to an enzyme.

In some embodiments, at least portions of a macromolecule analysis assay can be automated, such as a next generation protein assay using multiple binding agents and enzymatically or chemically mediated sequential information transfer. In some cases, the analysis assay is performed on immobilized protein molecules simultaneously bound by two or more cognate binding agents (e.g., antibodies). After multiple cognate antibody binding events, a combined primer extension and DNA nicking step is used to transfer information from the coding tags of bound antibodies to the recording tag. In some cases, polyclonal antibodies (or mixed population of monoclonal antibody) to multivalent epitopes on a protein can be used for the assay.

In some embodiments, the macromolecule comprises a polypeptide and the method includes performing a polypeptide analysis assay. In some embodiments, the sequence (or a portion of the sequence thereof) and/or the identity of a protein is determined using a polypeptide analysis assay. In some embodiments, the macromolecules may be processed or treated, such as with one or more enzymes and/or reagents. In some examples, the polypeptide analysis assay includes assessing at least a partial sequence or identity of the polypeptide using suitable techniques or procedures. For example, at least a partial sequence of the polypeptide can be assessed by N-terminal amino acid analysis or C-terminal amino acid analysis. In some embodiments, at least a partial sequence of the polypeptide can be assessed using a ProteoCode assay. In some examples, at least a partial sequence of the polypeptide can be assessed by the techniques or procedures disclosed and/or claimed in U.S. Provisional Patent Application Nos. 62/330,841, 62/339,071, 62/376,886, 62/579,844, 62/582,312, 62/583,448, 62/579,870, 62/579,840, and 62/582,916, and International Patent Publication Nos. WO 2017/192633, WO 2019/089836, WO 2019/089846, and WO 2019/089851.

In some embodiments, the provided automated methods are for generating a nucleic acid encoded library representation of the binding history of the macromolecule. This nucleic acid encoded library can be amplified, and analyzed using high-throughput next generation digital sequencing methods, enabling millions to billions of molecules to be analyzed per run. The creation of a nucleic acid encoded library of binding information is useful in another way in that it enables enrichment, subtraction, and normalization by DNA-based techniques that make use of hybridization. These DNA-based methods are easily and rapidly scalable and customizable, and more cost-effective than those available for direct manipulation of other types of macromolecule libraries, such as protein libraries. Thus, nucleic acid encoded libraries of binding information can be processed prior to sequencing by one or more techniques to enrich and/or subtract and/or normalize the representation of sequences. This enables information of maximum interest to be extracted much more efficiently, rapidly and cost-effectively from very large libraries whose individual members may initially vary in abundance over many orders of magnitude. Importantly, these nucleic-acid based techniques for manipulating library representation are orthogonal to more conventional methods, and can be used in combination with them.

In an exemplary workflow for analyzing peptides or polypeptides, the method generally includes contacting and binding of a binding agent comprising a coding tag to terminal amino acid (e.g., NTAA) of a peptide and transferring the binding agent's coding tag information to the recording tag associated with the peptide, thereby generating a first order extended recording tag. The terminal amino acid bound by the binding agent may be a chemically labeled or modified terminal amino acid. In some embodiments, the terminal amino acid (e.g., NTAA) is eliminated after the information from the coding tag is transferred. The terminal amino acid eliminated may be a chemically labeled or modified terminal amino acid. Removal of the NTAA by contacting with an enzyme or chemical reagents converts the penultimate amino acid of the peptide to a terminal amino acid. The polypeptide analysis may include one or more cycles of binding with additional binding agents to the terminal amino acid, transferring information from the additional binding agents to the extended nucleic acid thereby generating a higher order extended recording tag containing information from two or more coding tags, and eliminating the terminal amino acid in a cyclic manner. Additional binding, transfer, labeling, and removal, can occur as described above up to n amino acids to generate an n^{th} order extended nucleic acid, which collectively represent the peptide. In some embodiments, steps including the NTAA in the described exemplary approach can be performed instead with a C-terminal amino acid (CTAA). In some embodiments, the order of the steps in the process for a degradation-based peptide or polypeptide sequencing assay can be reversed or be performed in various orders. For example, in some embodiments, the terminal amino acid labeling can be conducted before and/or after the polypeptide is bound to the binding agent. In some embodiments, the workflow may include one or more wash steps before and/or after binding of the binding agents, transfer of information, labeling or modifying of the terminal amino acid, and/or removal of the terminal amino acid.

In some embodiments, the provided methods are for automated treatment of macromolecules from a sample for analysis using a degradation-like approach. In some cases, the approach uses a cyclic process including coding tag information transfer to a recording tag attached to the polypeptide, terminal amino acid elimination (e.g., NTAA elimination), and repeating the process in a cyclic manner.

In some embodiments, the polypeptide is attached, directly or indirectly, on a solid support. For example, the polypeptide is immobilized on a solid support via a capture agent. Either the protein or capture agent may co-localize or be labeled with a recording tag, and proteins with associated recording tags are directly immobilized on a solid support. Information can be transferred from the coding tag on the bound binding agent to a proximal recording tag using any suitable means including by ligation or primer extension. In one embodiment as depicted, the coding tag includes spacer that is complementary to the spacer in the recording tag and can be used to initiate a primer extension reaction to transfer recording tag information to the coding tag. The final extended recording tag is optionally flanked by universal priming sites to facilitate downstream amplification and/or DNA sequencing. The forward universal priming site (e.g., Illumina's P5-S1 sequence) can be part of the original recording tag design and the reverse universal priming site (e.g., Illumina's P7-S2′ sequence) can be added (e.g., by extension) to the final extended recording tag. This final step may be done independently of a binding agent.

In a workflow which includes binding of a natural or unmodified terminal amino acid, the analysis method includes contacting the polypeptide with a binding agent that is attached to a DNA coding tag. Upon binding of the binding agent to the NTAA of the polypeptide, information of the coding tag is transferred to the recording tag (e.g., via primer extension or ligation) to generate an extended recording tag. The NTAA is eliminated via chemical or biological (e.g., enzymatic) means to expose a new NTAA. In a workflow which includes a modified terminal amino acid, the first step includes labeling or modifying the N-terminal amino acid (NTAA) with a functionalization reagent to enable removal of the NTAA in a later step; the functionalizing reagent generates an NTAA residue containing a functionalization moiety (e.g., a modification or label). A second step includes contacting the polypeptide with a binding agent that is attached to a DNA coding tag. In some embodiments, the labeling or modification of the NTAA may be performed prior to or after contacting the polypeptide with a binding agent. Upon binding of the binding agent to the NTAA of the polypeptide, information of the coding tag is transferred to the recording tag (e.g., via primer extension or ligation) to generate an extended recording tag. Lastly, the functionalized NTAA is eliminated via chemical or biological (e.g., enzymatic) means to expose a new NTAA.

Using the provided automated treatment of macromolecules, the cycle described may be repeated “n” times to generate a final extended recording tag. In some embodiments, the order in the steps in the process for a degradation-based peptide polypeptide sequencing assay can be reversed or moved around. In some embodiments, the terminal amino acid functionalization can be conducted after the polypeptide is bound to a support. In some aspects, the analysis assay may include one or more additional steps, such as a wash step and/or treatment with other reagents. In some embodiments, the provided methods may be performed such that the C-terminal amino acid is modified, labeled, contacted by a binding agent, and/or eliminated from the polypeptide.

In some embodiments, the automated method includes a) providing a non-planar sample container comprising a sample comprising a macromolecule, e.g., a polypeptide, and an associated recording tag joined to a solid support to said apparatus; b) providing a binding agent and reagents for transferring information to separate reagent reservoirs of said apparatus, wherein at least one of said reagent reservoirs comprises a binding agent and at least one of said reagent reservoirs comprises reagents for transferring information; c) delivering the binding agent from the reagent reservoir to the sample container, wherein the binding agent comprises a coding tag with identifying information regarding the binding agent; and d) delivering the reagents for transferring information from the reagent reservoir to the sample container to transfer information from the coding tag of the binding agent to the recording tag to generate an extended recording tag. In some embodiments, the automated method further includes providing reagents for removing a terminal amino acid of a polypeptide to a separate reagent reservoir of said apparatus in step a) and step e) delivering the reagents for removing a terminal amino acid of a polypeptide from the reagent reservoir to the sample container to remove the terminal amino acid. In some aspects, the automated method further includes providing reagents for a capping reaction to a separate reagent reservoir of said apparatus in step a) and step f) delivering the reagents for a capping reaction from the reagent reservoir to the sample container. In some embodiments, the automated method further includes providing a reagent for modifying a terminal amino acid of a polypeptide to the reagent reservoir of said apparatus in step a) and delivering the reagent for modifying a terminal amino acid of a polypeptide to the sample container.

In some embodiments, macromolecules of the sample are associated with a recording tag. In some cases, the macromolecules of the sample are joined to a solid support, directly or indirectly. For example, the solid support can comprise a three-dimensional material (e.g., a gel matrix or a bead). In some example, a sample container is provided with the immobilized macromolecules of the sample which are associated with a recording tag. In some embodiments, the order in the steps for delivering the reagents to the sample container can be reversed or moved around. In one example, steps c), d), and e) are performed in order. In some cases, step f) is performed after steps b), c), d), and e). In some embodiments, the automated method further includes repeating steps c) to e) two or more times prior to performing step f).

In some embodiments, the automated method further includes providing a reagent for modifying (e.g., functionalizing) a terminal amino acid of a polypeptide to the reagent reservoir of an apparatus and delivering the reagent for modifying a terminal amino acid of a polypeptide to the sample container. In some embodiments, the reagent for modifying a terminal amino acid of a polypeptide comprises a chemical agent or an enzymatic agent. In some aspects, the reagent for modifying a terminal amino acid of a polypeptide is delivered to the sample container before step c), before step d), before step e), and/or before step f). In some cases, the reagent for modifying a terminal amino acid of a polypeptide is delivered to the sample container after step b) and before step c). In some cases, the delivery of the reagent for modifying a terminal amino acid of a polypeptide to the sample container is repeated two or more times, each time before the reagent(s) for removing a terminal amino acid of a polypeptide from the reagent reservoir tis delivered to the sample container to remove the terminal amino acid.

In some embodiments, the method further includes collecting the sample or a portion thereof after the capping reaction is performed in the sample container. In some embodiments, the sample or a portion thereof is collected in an automated manner and the collection is controlled by the control unit. For example, after the generation of a final extended recording tag, the sample is treated with a cleaving reagent to release the recording tag from the polypeptides in the sample, and the recording tags are collected.

A. Samples

In some aspects, the present disclosure relates to the automated treatment of macromolecules from a sample for analysis. A macromolecule can be a large molecule composed of smaller subunits. In certain embodiments, a macromolecule is a protein, a protein complex, polypeptide, peptide, nucleic acid molecule, carbohydrate, lipid, macrocycle, or a chimeric macromolecule. A macromolecule (e.g., protein, polypeptide, peptide) analyzed according the methods disclosed herein may be obtained from a suitable source or sample. In some embodiments, the macromolecules (e.g., proteins, polypeptides, or peptides) are obtained from a sample that is a biological sample. In some embodiments, the sample comprises but is not limited to, mammalian or human cells, yeast cells, and/or bacterial cells. In some embodiments, the sample contains cells that are from a sample obtained from a multicellular organism. For example, the sample may be isolated from an individual. In some embodiments, the sample may comprise a single cell type or multiple cell types. In some embodiments, the sample may be obtained from a mammalian organism or a human, for example by puncture, or other collecting or sampling procedures. In some embodiments, the sample comprises two or more cells.

In some embodiments, the biological sample may contain whole cells and/or live cells and/or cell debris. In some examples, a suitable source or sample, may include but is not limited to: biological samples, such as biopsy samples, cell cultures, cells (both primary cells and cultured cell lines), sample comprising cell organelles or vesicles, tissues and tissue extracts; of virtually any organism. For example, a suitable source or sample, may include but is not limited to: biopsy; fecal matter; bodily fluids (such as blood, whole blood, serum, plasma, urine, lymph, bile, aqueous humor, breast milk, cerumen (earwax), chyle, chyme, endolymph, perilymph, exudates, cerebrospinal fluid, interstitial fluid, aqueous or vitreous humor, colostrum, sputum, amniotic fluid, saliva, anal and vaginal secretions, gastric acid, gastric juice, lymph, mucus (including nasal drainage and phlegm), pericardial fluid, peritoneal fluid, pleural fluid, pus, rheum, saliva, sebum (skin oil), sputum, synovial fluid, perspiration and semen, a transudate, vomit and mixtures of one or more thereof, an exudate (e.g., fluid obtained from an abscess or any other site of infection or inflammation) or fluid obtained from a joint (normal joint or a joint affected by disease such as rheumatoid arthritis, osteoarthritis, gout or septic arthritis) of virtually any organism, with mammalian-derived samples, including microbiome-containing samples, being preferred and human-derived samples, including microbiome-containing samples, being particularly preferred; environmental samples (such as air, agricultural, water and soil samples); microbial samples including samples derived from microbial biofilms and/or communities, as well as microbial spores; tissue samples including tissue sections, research samples including extracellular fluids, extracellular supernatants from cell cultures, inclusion bodies in bacteria, cellular components including mitochondria and cellular periplasm. In some embodiments, the biological sample comprises a body fluid or is derived from a body fluid, wherein the body fluid is obtained from a mammal or a human. In some embodiments, the sample includes bodily fluids, or cell cultures from bodily fluids.

In some embodiments, the method includes obtaining and preparing macromolecules (e.g., polypeptides and proteins) from a single cell type or multiple cell types. In some embodiments, the sample comprises a population of cells. In some embodiments, the macromolecules (e.g., proteins, polypeptides, or peptides) are from a cellular or subcellular component, an extracellular vesicle, an organelle, or an organized subcomponent thereof. In some embodiments, the polypeptides are from one or more packaging of molecules (e.g., separate components of a single cell or separate components isolated from a population of cells, such as organelles or vesicles). The macromolecules (e.g., proteins, polypeptides, or peptides) may be from organelles, for example, mitochondria, nuclei, or cellular vesicles. In one embodiment, one or more specific types of single cells or subtypes thereof may be isolated. In some embodiments, the sample may include but are not limited to cellular organelles, (e.g., nucleus, golgi apparatus, ribosomes, mitochondria, endoplasmic reticulum, chloroplast, cell membrane, vesicles, etc.).

In certain embodiments, a macromolecule is a protein, a protein complex, a polypeptide, or peptide. Amino acid sequence information and post-translational modifications of a peptide, polypeptide, or protein are transduced into a nucleic acid encoded library that can be analyzed via next generation sequencing methods. A peptide may comprise L-amino acids, D-amino acids, or both. A peptide, polypeptide, protein, or protein complex may comprise a standard, naturally occurring amino acid, a modified amino acid (e.g., post-translational modification), an amino acid analog, an amino acid mimetic, or any combination thereof. In some embodiments, a peptide, polypeptide, or protein is naturally occurring, synthetically produced, or recombinantly expressed. In any of the aforementioned peptide embodiments, a peptide, polypeptide, protein, or protein complex may further comprise a post-translational modification. Standard, naturally occurring amino acids include Alanine (A or Ala), Cysteine (C or Cys), Aspartic Acid (D or Asp), Glutamic Acid (E or Glu), Phenylalanine (F or Phe), Glycine (G or Gly), Histidine (H or His), Isoleucine (I or Ile), Lysine (K or Lys), Leucine (L or Leu), Methionine (M or Met), Asparagine (N or Asn), Proline (P or Pro), Glutamine (Q or Gln), Arginine (R or Arg), Serine (S or Ser), Threonine (T or Thr), Valine (V or Val), Tryptophan (W or Trp), and Tyrosine (Y or Tyr). Non-standard amino acids include selenocysteine, pyrrolysine, and N-formylmethionine, 3-amino acids, homo-amino acids, Proline and Pyruvic acid derivatives, 3-substituted Alanine derivatives, Glycine derivatives, ring-substituted Phenylalanine and Tyrosine Derivatives, linear core amino acids, and N-methyl amino acids.

A post-translational modification (PTM) of a peptide, polypeptide, or protein may be a covalent modification or enzymatic modification. Examples of post-translation modifications include, but are not limited to, acylation, acetylation, alkylation (including methylation), biotinylation, butyrylation, carbamylation, carbonylation, deamidation, deiminiation, diphthamide formation, disulfide bridge formation, eliminylation, flavin attachment, formylation, gamma-carboxylation, glutamylation, glycylation, glycosylation (e.g., N-linked, O-linked, C-linked, phosphoglycosylation), glypiation, heme C attachment, hydroxylation, hypusine formation, iodination, isoprenylation, lipidation, lipoylation, malonylation, methylation, myristolylation, oxidation, palmitoylation, pegylation, phosphopantetheinylation, phosphorylation, prenylation, propionylation, retinylidene Schiff base formation, S-glutathionylation, S-nitrosylation, S-sulfenylation, selenation, succinylation, sulfination, ubiquitination, and C-terminal amidation. A post-translational modification includes modifications of the amino terminus and/or the carboxyl terminus of a peptide, polypeptide, or protein. Modifications of the terminal amino group include, but are not limited to, des-amino, N-lower alkyl, N-di-lower alkyl, and N-acyl modifications. Modifications of the terminal carboxy group include, but are not limited to, amide, lower alkyl amide, dialkyl amide, and lower alkyl ester modifications (e.g., wherein lower alkyl is C₁-C₄ alkyl). A post-translational modification also includes modifications, such as but not limited to those described above, of amino acids falling between the amino and carboxy termini of a peptide, polypeptide, or protein. Post-translational modification can regulate a protein's “biology” within a cell, e.g., its activity, structure, stability, or localization. For example, phosphorylation plays an important role in regulation of protein, particularly in cell signaling (Prabakaran et al., 2012, Wiley Interdiscip Rev Syst Biol Med 4: 565-583). In another example, the addition of sugars to proteins, such as glycosylation, has been shown to promote protein folding, improve stability, and modify regulatory function and the attachment of lipids to proteins enables targeting to the cell membrane. A post-translational modification can also include peptide, polypeptide, or protein modifications to include one or more detectable labels.

In certain embodiments, a peptide, polypeptide, or protein can be fragmented. Fragmentation may be performed prior to loading the sample onto the apparatus. In some cases, fragmentation may be performed in an automated manner using the apparatus. For example, the fragmented peptide can be obtained by fragmenting a protein from a sample, such as a biological sample. The peptide, polypeptide, or protein can be fragmented by any means known in the art, including fragmentation by a protease or endopeptidase. In some embodiments, fragmentation of a peptide, polypeptide, or protein is targeted by use of a specific protease or endopeptidase. A specific protease or endopeptidase binds and cleaves at a specific consensus sequence (e.g., TEV protease). In other embodiments, fragmentation of a peptide, polypeptide, or protein is non-targeted or random by use of a non-specific protease or endopeptidase. A non-specific protease may bind and cleave at a specific amino acid residue rather than a consensus sequence (e.g., proteinase K is a non-specific serine protease). In some embodiments, proteinases and endopeptidases, such as those known in the art, can be used to cleave a protein or polypeptide into smaller peptide fragments include proteinase K, trypsin, chymotrypsin, pepsin, thermolysin, thrombin, Factor Xa, furin, endopeptidase, papain, pepsin, subtilisin, elastase, enterokinase, Genenase™ I, Endoproteinase LysC, Endoproteinase AspN, Endoproteinase GluC, etc. (Granvogl et al., 2007, Anal Bioanal Chem 389: 991-1002). In certain embodiments, a peptide, polypeptide, or protein is fragmented by proteinase K, or optionally, a thermolabile version of proteinase K to enable rapid inactivation. In some cases, Proteinase K is stable in denaturing reagents, such as urea and SDS, and enables digestion of completely denatured proteins. Protein and polypeptide fragmentation into peptides can be performed before or after attachment of a DNA tag or DNA recording tag.

Chemical reagents can also be used to digest proteins into peptide fragments. A chemical reagent may cleave at a specific amino acid residue (e.g., cyanogen bromide hydrolyzes peptide bonds at the C-terminus of methionine residues). Chemical reagents for fragmenting polypeptides or proteins into smaller peptides include cyanogen bromide (CNBr), hydroxylamine, hydrazine, formic acid, BNPS-skatole [2-(2-nitrophenylsulfenyl)-3-methylindole], iodosobenzoic acid, ⋅NTCB+Ni (2-nitro-5-thiocyanobenzoic acid), etc.

In certain embodiments, following enzymatic or chemical cleavage, the resulting peptide fragments are approximately the same desired length, e.g., from about 10 amino acids to about 70 amino acids, from about 10 amino acids to about 60 amino acids, from about 10 amino acids to about 50 amino acids, about 10 to about 40 amino acids, from about 10 to about 30 amino acids, from about 20 amino acids to about 70 amino acids, from about 20 amino acids to about 60 amino acids, from about 20 amino acids to about 50 amino acids, about 20 to about 40 amino acids, from about 20 to about 30 amino acids, from about 30 amino acids to about 70 amino acids, from about 30 amino acids to about 60 amino acids, from about 30 amino acids to about 50 amino acids, or from about 30 amino acids to about 40 amino acids. A cleavage reaction may be monitored, preferably in real time, by spiking the protein or polypeptide sample with a short test FRET (fluorescence resonance energy transfer) peptide comprising a peptide sequence containing a proteinase or endopeptidase cleavage site. In the intact FRET peptide, a fluorescent group and a quencher group are attached to either end of the peptide sequence containing the cleavage site, and fluorescence resonance energy transfer between the quencher and the fluorophore leads to low fluorescence. Upon cleavage of the test peptide by a protease or endopeptidase, the quencher and fluorophore are separated giving a large increase in fluorescence. A cleavage reaction can be stopped when a certain fluorescence intensity is achieved, allowing a reproducible cleavage endpoint to be achieved.

A sample of macromolecules (e.g., peptides, polypeptides, or proteins) can undergo protein fractionation methods where proteins or peptides are separated by one or more properties such as cellular location, molecular weight, hydrophobicity, isoelectric point, or protein enrichment methods. In some embodiments, a subset of macromolecules (e.g., proteins) within a sample is fractionated such that a subset of the macromolecules is sorted from the rest of the sample. For example, the sample may undergo fractionation methods prior to attachment to a solid support. Alternatively, or additionally, protein enrichment methods may be used to select for a specific protein or peptide (see, e.g., Whiteaker et al., 2007, Anal. Biochem. 362:44-54, incorporated by reference in its entirety) or to select for a particular post translational modification (see, e.g., Huang et al., 2014. J. Chromatogr. A 1372:1-17, incorporated by reference in its entirety). Alternatively, a particular class or classes of proteins such as immunoglobulins, or immunoglobulin (Ig) isotypes such as IgG, can be affinity enriched or selected for analysis. In the case of immunoglobulin molecules, analysis of the sequence and abundance or frequency of hypervariable sequences involved in affinity binding are of particular interest, particularly as they vary in response to disease progression or correlate with healthy, immune, and/or or disease phenotypes. Overly abundant proteins can also be subtracted from the sample using standard immunoaffinity methods. Depletion of abundant proteins can be useful for plasma samples where over 80% of the protein constituent is albumin and immunoglobulins. Several commercial products are available for depletion of plasma samples of overly abundant proteins, including depletion spin columns that remove top 2-20 plasma proteins (Pierce, Agilent), or PROTIA and PROT20 (Sigma-Aldrich).

In certain embodiments, a protein sample dynamic range can be modulated by fractionating the protein sample using standard fractionation methods, including electrophoresis and liquid chromatography (Zhou et al., 2012, Anal Chem 84(2): 720-734), or partitioning the fractions into compartments (e.g., droplets) loaded with limited capacity protein binding beads/resin (e.g. hydroxylated silica particles) (McCormick, 1989, Anal Biochem 181(1): 66-74) and eluting bound protein. Excess protein in each compartmentalized fraction is washed away. Examples of electrophoretic methods include capillary electrophoresis (CE), capillary isoelectric focusing (CIEF), capillary isotachophoresis (CITP), free flow electrophoresis, gel-eluted liquid fraction entrapment electrophoresis (GELFrEE). Examples of liquid chromatography protein separation methods include reverse phase (RP), ion exchange (IE), size exclusion (SE), hydrophilic interaction, etc. Examples of compartment partitions include emulsions, droplets, microwells, physically separated regions on a flat substrate, etc. Exemplary protein binding beads/resins include silica nanoparticles derivatized with phenol groups or hydroxyl groups (e.g., StrataClean Resin from Agilent Technologies, RapidClean from LabTech, etc.). By limiting the binding capacity of the beads/resin, highly-abundant proteins eluting in a given fraction will only be partially bound to the beads, and excess proteins removed.

In some embodiments, a partition barcode is used which comprises assignment of a unique barcode to a subsampling of macromolecules from a population of macromolecules within a sample. This partition barcode may be comprised of identical barcodes arising from the partitioning of macromolecules within compartments labeled with the same barcode (e.g. a barcoded bead population in which multiple beads share the same barcode). The use of physical compartments effectively subsamples the original sample to provide assignment of partition barcodes. For instance, a set of beads labeled with 10,000 different compartment barcodes is provided. Furthermore, suppose in a given assay, that a population of 1 million beads are used in the assay. On average, there are 100 beads per compartment barcode (Poisson distribution). Further suppose that the beads capture an aggregate of 10 million macromolecules. On average, there are 10 macromolecules per bead, with 100 compartments per compartment barcode, there are effectively 1000 macromolecules per partition barcode (comprised of 100 compartment barcodes for 100 distinct physical compartments).

In another embodiment, single molecule partitioning and partition barcoding of polypeptides is accomplished by labeling polypeptides (chemically or enzymatically) with an amplifiable DNA UMI tag (e.g., recording tag) at the N or C terminus, or both. DNA tags are attached to the body of the polypeptide (internal amino acids) via non-specific photo-labeling or specific chemical attachment to reactive amino acids such as lysines. Information from the recording tag attached to the terminus of the peptide is transferred to the DNA tags via an enzymatic emulsion PCR (Williams et al., Nat Methods, (2006) 3(7):545-550; Schutze et al., Anal Biochem. (2011) 410(1):155-157) or emulsion in vitro transcription/reverse transcription (IVT/RT) step. In the preferred embodiment, a nanoemulsion is employed such that, on average, there is fewer than a single polypeptide per emulsion droplet with size from 50 nm-1000 nm (Nishikawa et al., J Nucleic Acids. (2012) 2012: 923214; Gupta et al., Soft Matter. (2016) 12(11):2826-41; Sole et al., Langmuir (2006, 22(20):8326-8332). Additionally, all the components of PCR are included in the aqueous emulsion mix including primers, dNTPs, Mg2+, polymerase, and PCR buffer. If IVT/RT is used, then the recording tag is designed with a T7/SP6 RNA polymerase promoter sequence to generate transcripts that hybridize to the DNA tags attached to the body of the polypeptide (Ryckelynck et al., RNA. (2015) 21(3):458-469). A reverse transcriptase (RT) copies the information from the hybridized RNA molecule to the DNA tag. In this way, emulsion PCR or IVT/RT can be used to effectively transfer information from the terminus recording tag to multiple DNA tags attached to the body of the polypeptide.

In some embodiments, a sample of macromolecules (e.g., peptides, polypeptides, or proteins) can be processed into a physical area or volume e.g., into a compartment. Various processing and/or labeling steps may be performed on the sample prior to loading the sample on the apparatus described in Section I. In some embodiments, the compartment separates or isolates a subset of macromolecules from a sample of macromolecules. In some examples, the compartment may be an aqueous compartment (e.g., microfluidic droplet), a solid compartment (e.g., picotiter well or microtiter well on a plate, tube, vial, bead), or a separated region on a surface. In some cases, a compartment may comprise one or more beads to which macromolecules may be immobilized. In some embodiments, macromolecules in a compartment is labeled with a compartment tag including a barcode. For example, the macromolecules in one compartment can be labeled with the same barcode or macromolecules in multiple compartments can be labeled with the same barcode. See e.g., Valihrach et al., Int J Mol Sci. 2018 Mar. 11; 19 (3). pii: E807. Encapsulation of cellular contents via gelation in beads is a useful approach to single cell analysis (Tamminen et al., Front Microbiol (2015) 6: 195; Spencer et al., ISME J (2016) 10(2): 427-436). Barcoding single cell droplets enables all components from a single cell to be labeled with the same identifier (Klein et al., Cell (2015) 161(5): 1187-1201; Zilionis et al., Nat Protoc (2017) 12(1): 44-73; International Patent Publication No. WO 2016/130704). Compartment barcoding can be accomplished in a number of ways including direct incorporation of unique barcodes into each droplet by droplet joining (Bio-Rad Laboratories), by introduction of barcoded beads into droplets (10× Genomics), or by combinatorial barcoding of components of the droplet post encapsulation and gelation using and split-pool combinatorial barcoding as described by Gunderson et al. (International Patent Publication No. WO 2016/130704, incorporated by reference in its entirety). A similar combinatorial labeling scheme can also be applied to nuclei (Vitak et al., Nat Methods (2017) 14(3):302-308).

The above droplet barcoding approaches have been used for DNA analysis but not for protein analysis. Adapting the above droplet barcoding platforms to work with proteins requires several innovative steps. The first is that barcodes are primarily comprised of DNA sequences, and this DNA sequence information needs to be conferred to the protein analyte. In the case of a DNA analyte, it is relatively straightforward to transfer DNA information onto a DNA analyte. In contrast, transferring DNA information onto proteins is more challenging, particularly when the proteins are denatured and digested into peptides for downstream analysis. This requires that each peptide be labeled with a compartment barcode. The challenge is that once the cell is encapsulated into a droplet, it is difficult to denature the proteins, protease digest the resultant polypeptides, and simultaneously label the peptides with DNA barcodes. Encapsulation of cells in polymer forming droplets and their polymerization (gelation) into porous beads, which can be brought up into an aqueous buffer, provides a vehicle to perform multiple different reaction steps, unlike cells in droplets (Tamminen et al., Front Microbiol (2015) 6: 195; Spencer et al., ISME J (2016) 10(2): 427-436; International Patent Publication No. WO 2016/130704). Preferably, the encapsulated proteins are crosslinked to the gel matrix to prevent their subsequent diffusion from the gel beads. This gel bead format allows the entrapped proteins within the gel to be denatured chemically or enzymatically, labeled with DNA tags, protease digested, and subjected to a number of other interventions. In some embodiments, encapsulation and lysis of a single cell in a gel matrix can be performed.

In some embodiments, the macromolecules (e.g., polypeptides) are joined to a support before performing a polypeptide analysis assay. In some cases, it is desirable to use a support with a large carrying capacity to immobilize a large number of macromolecules. In some embodiments, it is preferred to immobilize the macromolecules from the sample using a three-dimensional support (e.g., a porous matrix or a bead). For example, the preparation of the macromolecules in the sample including joining the macromolecule to a support may be performed prior to loading the sample on the apparatus. In some examples, the preparation of the macromolecules in the sample including joining the macromolecule to a recording tag may be performed prior to or after loading the sample on the apparatus. In some particular cases, a prepared sample (e.g., peptide-DNA conjugates) can be loaded onto the apparatus for the assay. Once loaded, the DNA tags of the sample of peptide-DNA conjugates are further used to immobilize the sample peptides on to the support in the sample container. In some embodiments, a plurality of proteins is attached to a support prior to the polypeptide analysis assay. In some embodiments, sample preparation steps such as attaching a recording tag to the macromolecules of the sample can be performed using the apparatus or performed in an automated fashion.

A support can be any solid or porous support including, but not limited to, a bead, a microbead, an array, a glass surface, a silicon surface, a plastic surface, a filter, a membrane, a PTFE membrane, nylon, a microtiter well, an ELISA plate, a spinning interferometry disc, a nitrocellulose membrane, a nitrocellulose-based polymer surface, a nanoparticle, or a microsphere. Materials for a solid support include but are not limited to acrylamide, agarose, cellulose, dextran, nitrocellulose, glass, gold, quartz, polystyrene, polyethylene vinyl acetate, polypropylene, polyester, polymethacrylate, polyacrylate, polyethylene, polyethylene oxide, polysilicates, polycarbonates, poly vinyl alcohol (PVA), Teflon, fluorocarbons, nylon, silicon rubber, silica, polyanhydrides, polyglycolic acid, polyvinylchloride, polylactic acid, polyorthoesters, functionalized silane, polypropylfumerate, collagen, glycosaminoglycans, polyamino acids, or any combination thereof. In certain embodiments, a solid support is a bead, for example, a polystyrene bead, a polymer bead, a polyacrylate bead, an agarose bead, a cellulose bead, a dextran bead, an acrylamide bead, a solid core bead, a porous bead, a paramagnetic bead, a glass bead, a silica-based bead, or a controlled pore bead, or any combinations thereof. In some specific embodiments, the solid support is a porous agarose bead. In some specific embodiments, the solid support is not a two-dimensional support.

In some embodiments, the support may comprise any suitable solid material, including porous and non-porous materials, to which a macromolecule, e.g., a polypeptide, can be associated directly or indirectly, by any means known in the art, including covalent and non-covalent interactions, or any combination thereof. In some cases, a suitable solid support may be compatible with the sample containers described in Section I.B. A solid support may be two-dimensional (e.g., planar surface) or three-dimensional (e.g., gel matrix or bead). A solid support can be any support surface including, but not limited to, a bead, a microbead, an array, a glass surface, a silicon surface, a plastic surface, a filter, a membrane, a PTFE membrane, a PTFE membrane, a nitrocellulose membrane, a nitrocellulose-based polymer surface, nylon, a microtiter well, an ELISA plate, a spinning interferometry disc, a polymer matrix, a nanoparticle, or a microsphere. Materials for a solid support include but are not limited to acrylamide, agarose, cellulose, dextran, nitrocellulose, glass, gold, quartz, polystyrene, polyethylene vinyl acetate, polypropylene, polyester, polymethacrylate, polyacrylate, polyethylene, polyethylene oxide, polysilicates, polycarbonates, poly vinyl alcohol (PVA), Teflon, fluorocarbons, nylon, silicon rubber, polyanhydrides, polyglycolic acid, polyvinylchloride, polylactic acid, polyorthoesters, functionalized silane, polypropylfumerate, collagen, glycosaminoglycans, polyamino acids, dextran, or any combination thereof. Solid supports further include thin film, membrane, bottles, dishes, fibers, woven fibers, shaped polymers such as tubes, particles, beads, microspheres, microparticles, or any combination thereof. For example, when solid surface is a bead, the bead can include, but is not limited to, a ceramic bead, a polystyrene bead, a polymer bead, a polyacrylate bead, a methylstyrene bead, an agarose bead, a cellulose bead, a dextran bead, an acrylamide bead, a solid core bead, a porous bead, a paramagnetic bead, a glass bead, or a controlled pore bead, a silica-based bead, or any combinations thereof. A bead may be spherical or an irregularly shaped. A bead or support may be porous. A bead's size may range from nanometers, e.g., 100 nm, to millimeters, e.g., 1 mm. In certain embodiments, beads range in size from about 0.2 micron to about 200 microns, or from about 0.5 micron to about 5 micron. In some embodiments, beads can be about 1, 1.5, 2, 2.5, 2.8, 3, 3.5, 4, 4.5, 5, 5.5, 6, 6.5, 7, 7.5, 8, 8.5, 9, 9.5, 10, 10.5, 15, or 20 m in diameter. In certain embodiments, “a bead” solid support may refer to an individual bead or a plurality of beads. In some embodiments, the solid surface is a nanoparticle. In certain embodiments, the nanoparticles range in size from about 1 nm to about 500 nm in diameter, for example, between about 1 nm and about 20 nm, between about 1 nm and about 50 nm, between about 1 nm and about 100 nm, between about 10 nm and about 50 nm, between about 10 nm and about 100 nm, between about 10 nm and about 200 nm, between about 50 nm and about 100 nm, between about 50 nm and about 150, between about 50 nm and about 200 nm, between about 100 nm and about 200 nm, or between about 200 nm and about 500 nm in diameter. In some embodiments, the nanoparticles can be about 10 nm, about 50 nm, about 100 nm, about 150 nm, about 200 nm, about 300 nm, or about 500 nm in diameter. In some embodiments, the nanoparticles are less than about 200 nm in diameter.

Various reactions may be used to attach the polypeptides to a support (e.g., a solid or a porous support). The polypeptides may be attached directly or indirectly to the support. In some cases, the polypeptide is attached to the support via a nucleic acid. Exemplary reactions include the copper catalyzed reaction of an azide and alkyne to form a triazole (Huisgen 1, 3-dipolar cycloaddition), strain-promoted azide alkyne cycloaddition (SPAAC), reaction of a diene and dienophile (Diels-Alder), strain-promoted alkyne-nitrone cycloaddition, reaction of a strained alkene with an azide, tetrazine or tetrazole, alkene and azide [3+2]cycloaddition, alkene and tetrazine inverse electron demand Diels-Alder (IEDDA) reaction (e.g., m-tetrazine (mTet) or phenyl tetrazine (pTet) and trans-cyclooctene (TCO)); or pTet and an alkene), alkene and tetrazole photoreaction, Staudinger ligation of azides and phosphines, and various displacement reactions, such as displacement of a leaving group by nucleophilic attack on an electrophilic atom (Horisawa 2014, Knall, Hollauf et al. 2014). Exemplary displacement reactions include reaction of an amine with: an activated ester; an N-hydroxysuccinimide ester; an isocyanate; an isothioscyanate, an aldehyde, an epoxide, or the like. In some embodiments, iEDDA click chemistry is used for immobilizing polypeptides to a solid support since it is rapid and delivers high yields at low input concentrations. In another embodiment, m-tetrazine rather than tetrazine is used in an iEDDA click chemistry reaction, as m-tetrazine has improved bond stability. In another embodiment, phenyl tetrazine (pTet) is used in an iEDDA click chemistry reaction. In one case, a polypeptide is labeled with a bifunctional click chemistry reagent, such as alkyne-NHS ester (acetylene-PEG-NHS ester) reagent or alkyne-benzophenone to generate an alkyne-labeled polypeptide. In some embodiments, an alkyne can also be a strained alkyne, such as cyclooctynes including Dibenzocyclooctyl (DBCO), etc.

In certain embodiments where multiple proteins are immobilized on the same solid support, the proteins can be spaced appropriately to accommodate methods of analysis to be used to assess the proteins. For example, it may be advantageous to space the proteins that optimally to allow a nucleic acid-based method for assessing and sequencing the proteins to be performed. In some embodiments, the method for assessing and sequencing the proteins involve a binding agent which binds to the protein and the binding agent comprises a coding tag with information that is transferred to a nucleic acid attached to the proteins (e.g., recording tag). In some cases, information transfer from a coding tag of a binding agent bound to one protein may reach a neighboring protein.

In some embodiments, the surface of the solid support is passivated (blocked). A “passivated” surface refers to a surface that has been treated with outer layer of material. Methods of passivating surfaces include standard methods from the fluorescent single molecule analysis literature, including passivating surfaces with polymer like polyethylene glycol (PEG) (Pan et al., 2015, Phys. Biol. 12:045006), polysiloxane (e.g., Pluronic F-127), star polymers (e.g., star PEG) (Groll et al., 2010, Methods Enzymol. 472:1-18), hydrophobic dichlorodimethylsilane (DDS)+self-assembled Tween-20 (Hua et al., 2014, Nat. Methods 11:1233-1236), diamond-like carbon (DLC), DLC+PEG (Stavis et al., 2011, Proc. Natl. Acad. Sci. USA 108:983-988), and zwitterionic moiety (e.g., U.S. Patent Application Publication US 2006/0183863). In addition to covalent surface modifications, a number of passivating agents can be employed as well including surfactants like Tween-20, polysiloxane in solution (Pluronic series), poly vinyl alcohol (PVA), and proteins like BSA and casein. Alternatively, density of macromolecules (e.g., proteins, polypeptide, or peptides) can be titrated on the surface or within the volume of a solid substrate by spiking a competitor or “dummy” reactive molecule when immobilizing the proteins, polypeptides or peptides to the solid substrate.

To control protein spacing on the solid support, the density of functional coupling groups for attaching the protein (e.g., TCO or carboxyl groups (COOH)) may be titrated on the substrate surface. In some embodiments, multiple proteins are spaced apart on the surface or within the volume (e.g., porous supports) of a solid support such that adjacent proteins are spaced apart at a distance of about 50 nm to about 500 nm, or about 50 nm to about 400 nm, or about 50 nm to about 300 nm, or about 50 nm to about 200 nm, or about 50 nm to about 100 nm. In some embodiments, multiple a proteins are spaced apart on the surface of a solid support with an average distance of at least 50 nm, at least 60 nm, at least 70 nm, at least 80 nm, at least 90 nm, at least 100 nm, at least 150 nm, at least 200 nm, at least 250 nm, at least 300 nm, at least 350 nm, at least 400 nm, at least 450 nm, or at least 500 nm. In some embodiments, multiple a proteins are spaced apart on the surface of a solid support with an average distance of at least 50 nm. In some embodiments, proteins are spaced apart on the surface or within the volume of a solid support such that, empirically, the relative frequency of inter- to intra-molecular events (e.g. transfer of information) is <1:10; <1:100; <1:1,000; or <1:10,000.

In some embodiments, the plurality of proteins is coupled on the solid support spaced apart at an average distance between two adjacent proteins which ranges from about 50 to 100 nm, from about 50 to 250 nm, from about 50 to 500 nm, from about 50 to 750 nm, from about 50 to 1,000 nm, from about 50 to 1,500 nm, from about 50 to 2,000 nm, from about 100 to 250 nm, from about 100 to 500 nm, from about 200 to 500 nm, from about 300 to 500 nm, from about 100 to 1000 nm, from about 500 to 600 nm, from about 500 to 700 nm, from about 500 to 800 nm, from about 500 to 900 nm, from about 500 to 1,000 nm, from about 500 to 2,000 nm, from about 500 to 5,000 nm, from about 1,000 to 5,000 nm, or from about 3,000 to 5,000 nm.

In some embodiments, appropriate spacing of the polypeptides on the solid support is accomplished by titrating the ratio of available attachment molecules on the substrate surface. In some examples, the substrate surface (e.g., bead surface) is functionalized with a carboxyl group (COOH) which is treated with an activating agent (e.g., activating agent is EDC and Sulfo-NHS). In some examples, the substrate surface (e.g., bead surface) comprises NHS moieties. In some embodiments, a mixture of mPEGₙ-NH₂ and NH₂—PEGₙ-mTet is added to the activated beads (wherein n is any number, such as 1-100). The ratio between the mPEG₃-NH₂ (not available for coupling) and NH₂—PEG₂₄-mTet (available for coupling) is titrated to generate an appropriate density of functional moieties available to attach the polypeptides on the substrate surface. In certain embodiments, the mean spacing between coupling moieties (e.g., NH₂-PEG₄-mTet) on the solid surface is at least 50 nm, at least 100 nm, at least 250 nm, or at least 500 nm. In some specific embodiments, the ratio of NH₂-PEGₙ-mTet to mPEG₃-NH₂ is about or greater than 1:1000, about or greater than 1:10,000, about or greater than 1:100,000, or about or greater than 1:1,000,000. In some further embodiments, the recording tag attaches to the NH₂-PEGₙ-mTet. In some embodiments, the spacing of the polypeptides on the solid support is achieved by controlling the concentration and/or number of available COOH or other functional groups on the solid support.

B. Recording Tag

As described herein, the macromolecule (e.g., protein or polypeptide) may be labeled with a DNA recording tag. In some embodiments, the sample is provided with a plurality of recording tags. In some aspects, a plurality of macromolecules in the sample is provided with recording tags. The recording tags may be associated or attached, directly or indirectly to the macromolecules using any suitable means. In some embodiments, a macromolecule may be associated with one or more recording tags. In some aspects, the recording tag may be any suitable sequenceable moiety to which identifying information can be transferred (e.g., information from one or more coding tags).

In some embodiments, at least one recording tag is associated or co-localized directly or indirectly with the macromolecule (e.g., polypeptide). In a particular embodiment, a single recording tag is attached to a polypeptide, such as via the attachment to a N- or C-terminal amino acid. In another embodiment, multiple recording tags are attached to the polypeptide, such as to the lysine residues or peptide backbone. In some embodiments, a polypeptide labeled with multiple recording tags is fragmented or digested into smaller peptides, with each peptide labeled on average with one recording tag.

A recording tag may comprise DNA, RNA, or polynucleotide analogs including PNA, gPNA, GNA, HNA, BNA, XNA, TNA, or a combination thereof. A recording tag may be single stranded, or partially or completely double stranded. A recording tag may have a blunt end or overhanging end. In certain embodiments, all or a substantial amount of the macromolecules (e.g., at least 50%, 55%, 60%, 65%, 70%, 75%, 80%, 85%, 90%, 95%, 96%, 97%, 98%, 99%, or 100%) within a sample are labeled with a recording tag. In other embodiments, a subset of macromolecules within a sample are labeled with recording tags. In a particular embodiment, a subset of macromolecules from a sample undergo targeted (analyte specific) labeling with recording tags. For example, targeted recording tag labeling of proteins may be achieved using target protein-specific binding agents (e.g., antibodies, aptamers, etc.). In some embodiments, the recording tags are attached to the macromolecules prior to providing the sample on a solid support. In some embodiments, the recording tags are attached to the macromolecules after providing the sample on the solid support.

In some embodiments, the recording tag may comprise other nucleic acid components. In some embodiments, the recording tag may comprise a unique molecular identifier, a compartment tag, a partition barcode, sample barcode, a fraction barcode, a spacer sequence, a universal priming site, or any combination thereof. In some embodiments, the recording tag can further comprise other information including information from a macromolecule analysis assay, such as binder identifier (e.g., from a coding tag), cycle identifier (e.g., from a coding tag), etc. In some embodiments, the recording tag may comprise a blocking group, such as at the 3′-terminus of the recording tag. In some cases, the 3′-terminus of the recording tag is blocked to prevent extension of the recording tag by a polymerase.

In some embodiments, the recording tag can include a sample identifying barcode. A sample barcode is useful in the multiplexed analysis of a set of samples in a single reaction vessel or immobilized to a single solid substrate or collection of solid substrates (e.g., a planar slide, population of beads contained in a single tube or vessel, etc.). For example, macromolecules from many different samples can be labeled with recording tags with sample-specific barcodes, and then all the samples pooled together prior to immobilization to a solid support, cyclic binding of the binding agent, and recording tag analysis. Alternatively, the samples can be kept separate until after creation of a DNA-encoded library, and sample barcodes attached during PCR amplification of the DNA-encoded library, and then mixed together prior to sequencing. This approach could be useful when assaying analytes (e.g., proteins) of different abundance classes.

In certain embodiments, a recording tag comprises an optional, unique molecular identifier (UMI), which provides a unique identifier tag for each macromolecules (e.g., polypeptide) to which the UMI is associated with. A UMI can be about 3 to about 40 bases, about 3 to about 30 bases, about 3 to about 20 bases, or about 3 to about 10 bases, or about 3 to about 8 bases. In some embodiments, a UMI is about 3 bases, 4 bases, 5 bases, 6 bases, 7 bases, 8 bases, 9 bases, 10 bases, 11 bases, 12 bases, 13 bases, 14 bases, 15 bases, 16 bases, 17 bases, 18 bases, 19 bases, 20 bases, 25 bases, 30 bases, 35 bases, or 40 bases in length. A UMI can be used to de-convolute sequencing data from a plurality of extended recording tags to identify sequence reads from individual macromolecules. In some embodiments, within a library of macromolecules, each macromolecule is associated with a single recording tag, with each recording tag comprising a unique UMI. In other embodiments, multiple copies of a recording tag are associated with a single macromolecule, with each copy of the recording tag comprising the same UMI. In some embodiments, a UMI has a different base sequence than the spacer or encoder sequences within the binding agents' coding tags to facilitate distinguishing these components during sequence analysis. In some embodiments, the UMI may provide function as a location identifier and also provide information in the macromolecule analysis assay. For example, the UMI may be used to identify molecules that are identical by descent, and therefore originated from the same initial molecule. In some aspects, this information can be used to correct for variations in amplification, and to detect and correct sequencing errors.

In some embodiments, the recording tag comprises a spacer polymer. In certain embodiments, a recording tag comprises a spacer at its terminus, e.g., 3′ end. As used herein reference to a spacer sequence in the context of a recording tag includes a spacer sequence that is identical to the spacer sequence associated with its cognate binding agent, or a spacer sequence that is complementary to the spacer sequence associated with its cognate binding agent. The terminal, e.g., 3′, spacer on the recording tag permits transfer of identifying information of a cognate binding agent from its coding tag to the recording tag during the first binding cycle (e.g., via annealing of complementary spacer sequences for primer extension or sticky end ligation). In one embodiment, the spacer sequence is about 1-20 bases in length, about 2-12 bases in length, or 5-10 bases in length. The length of the spacer may depend on factors such as the temperature and reaction conditions of the primer extension reaction for transferring coding tag information to the recording tag.

In some embodiments, the recording tags associated with a library of polypeptides share a common spacer sequence. In other embodiments, the recording tags associated with a library of polypeptides have binding cycle specific spacer sequences that are complementary to the binding cycle specific spacer sequences of their cognate binding agents. In some aspects, the spacer sequence in the recording tag is designed to have minimal complementarity to other regions in the recording tag; likewise, the spacer sequence in the coding tag should have minimal complementarity to other regions in the coding tag. In some cases, the spacer sequence of the recording tags and coding tags should have minimal sequence complementarity to components such unique molecular identifiers, barcodes (e.g., compartment, partition, sample, spatial location), universal primer sequences, encoder sequences, cycle specific sequences, etc. present in the recording tags or coding tags.

In certain embodiments, a recording tag comprises a universal priming site, e.g., a forward or 5′ universal priming site. A universal priming site is a nucleic acid sequence that may be used for priming a library amplification reaction and/or for sequencing. A universal priming site may include, but is not limited to, a priming site for PCR amplification, flow cell adaptor sequences that anneal to complementary oligonucleotides on flow cell surfaces (e.g., Illumina next generation sequencing), a sequencing priming site, or a combination thereof. A universal priming site can be about 10 bases to about 60 bases. In some embodiments, a universal priming site comprises an Illumina P5 primer (5′-AATGATACGGCGACCACCGA-3′—SEQ ID NO:1) or an Illumina P7 primer (5′-CAAGCAGAAGACGGCATACGAGAT-3′ —SEQ ID NO:2).

In certain embodiments, a recording tag comprises a compartment tag. In some embodiments, the compartment tag is a component within a recording tag. In some embodiments, the recording tag can also include a barcode which represents a compartment tag in which a compartment, such as a droplet, microwell, physical region on a solid support, etc. is assigned a unique barcode. The association of a compartment with a specific barcode can be achieved in any number of ways such as by encapsulating a single barcoded bead in a compartment, e.g., by direct merging or adding a barcoded droplet to a compartment, by directly printing or injecting a barcode reagents to a compartment, etc. The barcode reagents within a compartment are used to add compartment-specific barcodes to the macromolecule or fragments thereof within the compartment. Applied to protein partitioning into compartments, the barcodes can be used to map analyzed peptides back to their originating protein molecules in the compartment. This can greatly facilitate protein identification. Compartment barcodes can also be used to identify protein complexes. In other embodiments, multiple compartments that represent a subset of a population of compartments may be assigned a unique barcode representing the subset. In some embodiments, the recording tag comprises fraction barcode which contains identifying information for the macromolecules within a fraction.

In some embodiments, the one or more tags or information of the one or more tags are transferred to the recording tag (e.g., via primer extension or ligation) to extend the recording tag. In some embodiments, one or more of the tags (e.g., compartment tag, a partition barcode, sample barcode, a fraction barcode, etc.) further comprise a functional moiety capable of reacting with an internal amino acid, the peptide backbone, or N-terminal amino acid on the plurality of protein complexes, proteins, or polypeptides. In some embodiments, the functional moiety is a click chemistry moiety, an aldehyde, an azide/alkyne, or a maleimide/thiol, or an epoxide/nucleophile, an inverse electron demand Diels-Alder (iEDDA) group, or a moiety for a Staudinger reaction. In some specific embodiments, a plurality of compartment tags is formed by printing, spotting, ink-jetting the compartment tags into the compartment, or a combination thereof. In some embodiments, the tag is attached to a polypeptide to link the tag to the macromolecule via a polypeptide-polypeptide linkage. In some embodiments, the tag-attached polypeptide comprises a protein ligase recognition sequence.

In certain embodiments, a peptide or polypeptide macromolecule can be immobilized to a solid support by an affinity capture reagent (and optionally covalently crosslinked), wherein the recording tag is associated with the affinity capture reagent directly, or alternatively, the macromolecule can be directly immobilized to the solid support with a recording tag. In one embodiment, the macromolecule is attached to a bait nucleic acid which hybridizes to a capture nucleic acid and is ligated to a capture nucleic acid which comprises a reactive coupling moiety for attaching to the solid support. In some examples, the bait or capture nucleic acid may serve as a recording tag to which information regarding the polypeptide can be transferred. In some embodiments, the macromolecule is attached to a bait nucleic acid to form a nucleic acid-macromolecule chimera. In some embodiments, the immobilization methods comprise bringing the nucleic acid-macromolecule chimera into proximity with a solid support by hybridizing the bait nucleic acid to a capture nucleic acid attached to the solid support, and covalently coupling the nucleic acid-macromolecule chimera to the solid support. In some cases, the nucleic acid-macromolecule chimera is coupled indirectly to the solid support, such as via a linker. In some embodiments, a plurality of the nucleic acid-macromolecule chimeras is coupled on the solid support and any adjacently coupled nucleic acid-macromolecule chimeras are spaced apart from each other at an average distance of about 50 nm or greater.

In some embodiments, the density or number of macromolecules provided with a recording tag is controlled or titrated. In some examples, the desired spacing, density, and/or amount of recording tags in the sample may be titrated by providing a diluted or controlled number of recording tags. In some examples, the desired spacing, density, and/or amount of recording tags may be achieved by spiking a competitor or “dummy” competitor molecule when providing, associating, and/or attaching the recording tags. In some cases, the “dummy” competitor molecule reacts in the same way as a recording tag being associated or attached to a macromolecule in the sample but the competitor molecule does not function as a recording tag. In some specific examples, if a desired density is 1 functional recording tag per 1,000 available sites for attachment in the sample, then spiking in 1 functional recording tag for every 1,000 “dummy” competitor molecules is used to achieve the desired spacing. In some examples, the ratio of functional recording tags is adjusted based on the reaction rate of the functional recording tags compared to the reaction rate of the competitor molecules.

In some examples, the labeling of the macromolecule with a recording tag is performed using standard amine coupling chemistries. For example, the e-amino group (e.g., of lysine residues) and the N-terminal amino group may be susceptible to labeling with amine-reactive coupling agents, depending on the pH of the reaction (Mendoza et al., Mass Spectrom Rev (2009) 28(5): 785-815). In a particular embodiment, the recording tag comprises a reactive moiety (e.g., for conjugation to a solid surface, a multifunctional linker, or a macromolecule), a linker, a universal priming sequence, a barcode (e.g., compartment tag, partition barcode, sample barcode, fraction barcode, or any combination thereof), an optional UMI, and a spacer (Sp) sequence for facilitating information transfer to/from a coding tag. In another embodiment, the protein can be first labeled with a universal DNA tag, and the barcode-Sp sequence (representing a sample, a compartment, a physical location on a slide, etc.) are attached to the protein later through and enzymatic or chemical coupling step. A universal DNA tag comprises a short sequence of nucleotides that are used to label a protein or polypeptide macromolecule and can be used as point of attachment for a barcode (e.g., compartment tag, recording tag, etc.). For example, a recording tag may comprise at its terminus a sequence complementary to the universal DNA tag. In certain embodiments, a universal DNA tag is a universal priming sequence. Upon hybridization of the universal DNA tags on the labeled protein to complementary sequence in recording tags (e.g., bound to beads), the annealed universal DNA tag may be extended via primer extension, transferring the recording tag information to the DNA tagged protein. In a particular embodiment, the protein is labeled with a universal DNA tag prior to proteinase digestion into peptides. The universal DNA tags on the labeled peptides from the digest can then be converted into an informative and effective recording tag.

The recording tags may comprise a reactive moiety for a cognate reactive moiety present on the target macromolecule, e.g., the target protein, (e.g., click chemistry labeling, photoaffinity labeling). For example, recording tags may comprise an azide moiety for interacting with alkyne-derivatized proteins, or recording tags may comprise a benzophenone for interacting with native proteins, etc. Upon binding of the target protein by the target protein specific binding agent, the recording tag and target protein are coupled via their corresponding reactive moieties. After the target protein is labeled with the recording tag, the target-protein specific binding agent may be removed by digestion of the DNA capture probe linked to the target-protein specific binding agent. For example, the DNA capture probe may be designed to contain uracil bases, which are then targeted for digestion with a uracil-specific excision reagent (e.g., USER™), and the target-protein specific binding agent may be dissociated from the target protein. In some embodiments, other types of linkages besides hybridization can be used to link the recording tag to a macromolecule. A suitable linker can be attached to various positions of the recording tag, such as the 3′ end, at an internal position, or within the linker attached to the 5′ end of the recording tag.

C. Cyclic Transfer of Coding Tag Information to Recording Tag

In some embodiments, the macromolecule analysis assay (e.g., polypeptide analysis assay) includes extending the recording tag associated with the macromolecule, e.g., the polypeptide, by transferring identifying information from one or more coding tags to the recording tag. In the methods described herein, upon binding of a binding agent to a macromolecule, e.g., a protein or peptide, identifying information of its linked coding tag is transferred to the recording tag (e.g., recording tag) associated with the polypeptide or peptide, thereby generating an extended recording tag. In some embodiments, the recording tag further comprises barcodes and/or other nucleic acid components. In particular embodiments, the identifying information from the coding tag of the binding agent is transferred to the recording tag or added to any existing barcodes (or other nucleic acid components) attached thereto. The transfer of the identifying information may be performed using extension or ligation. In some embodiments, a spacer is added to the end of the recording tag, and the spacer comprises a sequence that is capable of hybridizing with a sequence on the coding tag to facilitate the transfer of the identifying information from the coding tag. In some embodiments, the identifying information from the coding tag comprises information regarding the identity of the one or more amino acid(s) on the peptide or polypeptide bound by the binding agent.

In some embodiments, in a cyclic manner, the terminal amino acid (e.g., N-terminal amino acid) of each polypeptide or peptide is labeled (e.g., phenylthiocarbamoyl (PTC), modified-PTC, Cbz, dinitrophenyl (DNP) moiety, sulfonyl nitrophenyl (SNP), acetyl, guanidinyl, amino guanidinyl, heterocyclic methanimine). In some cases, the labeling of the terminal amino acid (e.g., N-terminal amino acid) can be performed before or after the binding of a binding agent to the peptide or polypeptide. The N-terminal amino acid (or labeled N-terminal amino acid, e.g., PTC-NTAA, Cbz-NTAA, DNP-NTAA, SNP-NTAA, acetyl-NTAA, guanidinylated-NTAA, amino guanidinyl-NTAA, heterocyclic methanimine-NTAA) of each immobilized polypeptide or peptide is bound by a cognate NTAA binding agent which is attached to a coding tag, and identifying information from the coding tag associated with the bound NTAA binding agent is transferred to the bait or capture nucleic acid associated with the immobilized polypeptide or peptide analyte, thereby generating an extended nucleic acid containing information from the coding tag.

In some embodiments, the bound binding agents are released from the polypeptide after identifying information from the coding tag of the binding agent is transferred to the recording tag. In some embodiments, the one or more binding agents are removed from the polypeptide after identifying information from the coding tag of the binding agent is transferred to the recording tag. In some aspects, after identifying information from the coding tag of the binding agent is transferred to the recording tag, a wash step is performed.

In some embodiments, the binding agents are associated with a coding tag and other optional nucleic acid components. The coding tag associated with the binding agent is or comprises a polynucleotide with any suitable length, e.g., a nucleic acid molecule of about 2 bases to about 100 bases, including any integer including 2 and 100 and in between, that comprises identifying information for its associated binding agent. A “coding tag” may also be made from a “sequenceable polymer” (see, e.g., Niu et al., 2013, Nat. Chem. 5:282-292; Roy et al., 2015, Nat. Commun. 6:7237; Lutz, 2015, Macromolecules 48:4759-4767; each of which are incorporated by reference in its entirety). A coding tag may comprise an encoder sequence or a sequence with identifying information, which is optionally flanked by one spacer on one side or optionally flanked by a spacer on each side. A coding tag may also be comprised of an optional UMI and/or an optional binding cycle-specific barcode. A coding tag may be single stranded or double stranded. A double stranded coding tag may comprise blunt ends, overhanging ends, or both. A coding tag may refer to the coding tag that is directly attached to a binding agent, to a complementary sequence hybridized to the coding tag directly attached to a binding agent (e.g., for double stranded coding tags), or to coding tag information present in an extended nucleic acid on the recording tag. In certain embodiments, a coding tag may further comprise a binding cycle specific spacer or barcode, a unique molecular identifier, a universal priming site, or any combination thereof.

Coding tag information associated with a specific binding agent may be transferred to a recording tag using a variety of methods. In any of the preceding embodiments, the transfer of identifying information (e.g., from a coding tag to a recording tag) can be accomplished by ligation (e.g., an enzymatic or chemical ligation, a splint ligation, a sticky end ligation, a single-strand (ss) ligation such as a ssDNA ligation, or any combination thereof), a polymerase-mediated reaction (e.g., primer extension of single-stranded nucleic acid or double-stranded nucleic acid), or any combination thereof.

In certain embodiments, information of a coding tag is transferred to a recording tag via primer extension (See e.g., Chan et al. (2015) Curr Opin Chem Biol 26: 55-61). A spacer sequence on the 3′-terminus of a recording tag or an extended recording tag anneals with complementary spacer sequence on the 3′ terminus of a coding tag and a polymerase (e.g., strand-displacing polymerase) extends the recording tag sequence, using the annealed coding tag as a template. In some embodiments, oligonucleotides complementary to coding tag encoder sequence and 5′ spacer can be pre-annealed to the coding tags to prevent hybridization of the coding tag to internal encoder and spacer sequences present in an extended recording tag. The 3′ terminal spacer, on the coding tag, remaining single stranded, preferably binds to the terminal 3′ spacer on the recording tag. In other embodiments, a nascent recording tag can be coated with a single stranded binding protein to prevent annealing of the coding tag to internal sites. Alternatively, the nascent recording tag can also be coated with RecA (or related homologues such as uvsX) to facilitate invasion of the 3′ terminus into a completely double stranded coding tag (Bell et al., 2012, Nature 491:274-278). This configuration prevents the double stranded coding tag from interacting with internal recording tag elements, yet is susceptible to strand invasion by the RecA coated 3′ tail of the extended recording tag (Bell et al., 2015, Elife 4: e08646). The presence of a single-stranded binding protein can facilitate the strand displacement reaction.

In some embodiments, a DNA polymerase that is used for primer extension possesses strand-displacement activity and has limited or is devoid of 3′-5 exonuclease activity. Several of many examples of such polymerases include Klenow exo- (Klenow fragment of DNA Pol 1), T4 DNA polymerase exo-, T7 DNA polymerase exo (Sequenase 2.0), Pfu exo-, Vent exo-, Deep Vent exo-, Bst DNA polymerase large fragment exo-, Bca Pol, 9° N Pol, and Phi29 Pol exo-. In a preferred embodiment, the DNA polymerase is active at room temperature and up to 45° C. In another embodiment, a “warm start” version of a thermophilic polymerase is employed such that the polymerase is activated and is used at about 40° C.-50° C. An exemplary warm start polymerase is Bst 2.0 Warm Start DNA Polymerase (New England Biolabs).

Additives useful in strand-displacement replication include any of a number of single-stranded DNA binding proteins (SSB proteins) of bacterial, viral, or eukaryotic origin, such as SSB protein of E. coli, phage T4 gene 32 product, phage T7 gene 2.5 protein, phage Pf3 SSB, replication protein A RPA32 and RPA14 subunits (Wold, Annu. Rev. Biochem. (1997) 66:61-92); other DNA binding proteins, such as adenovirus DNA-binding protein, herpes simplex protein ICP8, BMRF1 polymerase accessory subunit, herpes virus UL29 SSB-like protein; any of a number of replication complex proteins known to participate in DNA replication, such as phage T7 helicase/primase, phage T4 gene 41 helicase, E. coli Rep helicase, E. coli recBCD helicase, recA, E. coli and eukaryotic topoisomerases (Annu Rev Biochem. (2001) 70:369-413).

Mis-priming or self-priming events, such as when the terminal spacer sequence of the recoding tag primes extension self-extension may be minimized by inclusion of single stranded binding proteins (T4 gene 32, E. coli SSB, etc.), DMSO (1-10%), formamide (1-10%), BSA (10-100 ug/ml), TMACl (1-5 mM), ammonium sulfate (10-50 mM), betaine (1-3 M), glycerol (5-40%), or ethylene glycol (5-40%), in the primer extension reaction.

Most type A polymerases are devoid of 3′ exonuclease activity (endogenous or engineered removal), such as Klenow exo-, T7 DNA polymerase exo- (Sequenase 2.0), and Taq polymerase catalyzes non-templated addition of a nucleotide, preferably an adenosine base (to lesser degree a G base, dependent on sequence context) to the 3′ blunt end of a duplex amplification product. For Taq polymerase, a 3′ pyrimidine (C>T) minimizes non-templated adenosine addition, whereas a 3′ purine nucleotide (G>A) favours non-templated adenosine addition. In some embodiments, using Taq polymerase for primer extension, placement of a thymidine base in the coding tag between the spacer sequence distal from the binding agent and the adjacent barcode sequence (e.g., encoder sequence or cycle specific sequence) accommodates the sporadic inclusion of a non-templated adenosine nucleotide on the 3′ terminus of the spacer sequence of the recording tag. In this manner, the extended recording tag associated with the immobilized peptide (with or without a non-templated adenosine base) can anneal to the coding tag and undergo primer extension.

Alternatively, addition of non-templated base can be reduced by employing a mutant polymerase (mesophilic or thermophilic) in which non-templated terminal transferase activity has been greatly reduced by one or more point mutations, especially in the O-helix region (see U.S. Pat. No. 7,501,237) (Yang et al., Nucleic Acids Res. (2002) 30(19): 4314-4320). Pfu exo-, which is 3′ exonuclease deficient and has strand-displacing ability, also does not have non-templated terminal transferase activity.

In some embodiments, various conditions for one or more steps of the method may be modified by one skilled in the art as appropriate for automation, or for compatible use with an apparatus. For example, the temperature for contacting of the binding agents to the macromolecules or for hybridization of the spacer sequences on the recording tag and coding tag can be increased or decreased to modify specificity or stringency of the interactions. In some embodiments, to minimize non-specific interaction of the coding tag labeled binding agents in solution with the nucleic acids of immobilized proteins, competitor (also referred to as blocking) oligonucleotides complementary to nucleic acids containing spacer sequences (e.g., on the recording tag) can be added to binding reactions to minimize non-specific interactions. In some embodiments, the blocking oligonucleotides contain a sequence that is complementary to the coding tag or a portion thereof attached to the binding agent. In some embodiments, blocking oligonucleotides are relatively short. In some embodiments, the blocking oligonucleotide is directly or indirectly attached to the coding tag. In some examples, the coding tag comprises a hairpin nucleic acid, and the hairpin includes a sequence that is complementary to a spacer and/or barcode of the coding tag. Excess competitor oligonucleotides are washed from the binding reaction prior to primer extension, which effectively dissociates the annealed competitor oligonucleotides from the nucleic acids on the recording tag, especially when exposed to slightly elevated temperatures (e.g., 30-50° C.). In some embodiments, blocking oligonucleotides may comprise a terminator nucleotide at its 3′ end to prevent primer extension.

In certain embodiments, the annealing of the spacer sequence on the recording tag to the complementary spacer sequence on the coding tag is metastable under the primer extension reaction conditions (i.e., the annealing Tm is similar to the reaction temperature). This allows the spacer sequence of the coding tag to displace any blocking oligonucleotide annealed to the spacer sequence of the recording tag (or extensions thereof).

Self-priming/mis-priming events initiated by self-annealing of the terminal spacer sequence of the extended recording tag with internal regions of the extended recording tag may be minimized by including pseudo-complementary bases in the recording/extended recording tag (Lahoud et al., Nucleic Acids Res. (2008) 36:3409-3419), (Hoshika et al., Angew Chem Int Ed Engl (2010) 49(32): 5554-5557). Pseudo-complementary bases show significantly reduced hybridization affinities for the formation of duplexes with each other due to the presence of chemical modification. However, many pseudo-complementary modified bases can form strong base pairs with natural DNA or RNA sequences. In certain embodiments, the coding tag spacer sequence is comprised of multiple A and T bases, and commercially available pseudo-complementary bases 2-aminoadenine and 2-thiothymine are incorporated in the recording tag using phosphoramidite oligonucleotide synthesis. Additional pseudocomplementary bases can be incorporated into the extended recording tag during primer extension by adding pseudo-complementary nucleotides to the reaction (Gamper et al., Biochemistry. (2006) 45(22):6978-6986).

Coding tag information associated with a specific binding agent may be transferred to a nucleic acid on the recording tag associated with the immobilized polypeptide or peptide via ligation. Ligation may be a blunt end ligation or sticky end ligation. Ligation may be an enzymatic ligation reaction. Examples of ligases include, but are not limited to CV DNA ligase, T4 DNA ligase, T7 DNA ligase, T3 DNA ligase, Taq DNA ligase, E. coli DNA ligase, 9° N DNA ligase, Electroligase® (See e.g., U.S. Patent Publication No. US20140378315). Alternatively, a ligation may be a chemical ligation reaction. As illustrated in International Patent Publication No. WO 2017/192633, a spacer-less ligation is accomplished by using hybridization of a “recording helper” sequence with an arm on the coding tag. The annealed complement sequences are chemically ligated using standard chemical ligation or “click chemistry” (Gunderson et al., Genome Res (1998) 8(11): 1142-1153; Peng et al., European J Org Chem (2010) (22): 4194-4197; El-Sagheer et al., Proc Natl Acad Sci USA (2011) 108(28): 11338-11343; El-Sagheer et al., Org Biomol Chem (2011) 9(1): 232-235; Sharma et al., Anal Chem (2012) 84(14): 6104-6109; Roloff et al., Bioorg Med Chem (2013) 21(12): 3458-3464; Litovchick et al., Artif DNA PNA XNA (2014) 5(1): e27896; Roloff et al., Methods Mol Biol (2014) 1050:131-141).

In another embodiment, transfer of PNAs can be accomplished with chemical ligation using published techniques. The structure of PNA is such that it has a 5′ N-terminal amine group and an unreactive 3′ C-terminal amide. Chemical ligation of PNA requires that the termini be modified to be chemically active. This is typically done by derivatizing the 5′ N-terminus with a cysteinyl moiety and the 3′ C-terminus with a thioester moiety. Such modified PNAs easily couple using standard native chemical ligation conditions (Roloff et al., (2013) Bioorgan. Med. Chem. 21:3458-3464).

In some embodiments, coding tag information can be transferred using topoisomerase. Topoisomerase can be used be used to ligate a topo-charged 3′ phosphate on the recording tag (or extensions thereof or any nucleic acids attached) to the 5′ end of the coding tag, or complement thereof (Shuman et al., 1994, J. Biol. Chem. 269:32678-32684).

The extended recording tag can be any nucleic acid molecule or sequenceable polymer molecule (see, e.g., Niu et al., 2013, Nat. Chem. 5:282-292; Roy et al., 2015, Nat. Commun. 6:7237; Lutz, 2015, Macromolecules 48:4759-4767; each of which are incorporated by reference in its entirety) that comprises identifying information for a polypeptide to which it is associated. In some examples, the extended recording tag may comprise a unique molecular identifier, a compartment tag, a partition barcode, sample barcode, a fraction barcode, a spacer sequence, a universal priming site, or any combinations thereof. In certain embodiments, after a binding agent binds a polypeptide, information from a coding tag linked to a binding agent can be transferred to the nucleic acid associated with the polypeptide while the binding agent is bound to the polypeptide. In some examples, the final extended recording tag containing information from one or more binding agents is optionally flanked by universal priming sites to facilitate downstream amplification and/or DNA sequencing. The forward universal priming site (e.g., Illumina's P5-S1 sequence) can be part of the original design of the recording tag and the reverse universal priming site (e.g., Illumina's P7-S2′ sequence) can be added as a final step in the extension of the nucleic acid. In some embodiments, the addition of forward and reverse priming sites can be done independently of a binding agent.

An extended nucleic acid associated with the macromolecule, e.g., the peptide, with identifying information from the coding tag may comprise information from a binding agent's coding tag representing each binding cycle performed. However, in some cases, an extended nucleic acid may also experience a “missed” binding cycle, e.g., if a binding agent fails to bind to the polypeptide, because the coding tag was missing, damaged, or defective, because the primer extension reaction failed. Even if a binding event occurs, transfer of information from the coding tag may be incomplete or less than 100% accurate, e.g., because a coding tag was damaged or defective, because errors were introduced in the primer extension reaction). Thus, an extended nucleic acid may represent 100%, or up to 95%, 90%, 85%, 80%, 75%, 70%, 65%, 60%, 65%, 55%, 50%, 45%, 40%, 35%, 30%, or any subrange thereof, of binding events that have occurred on its associated polypeptide. Moreover, the coding tag information present in the extended nucleic acid may have at least 30%, 35%, 40%, 45%, 50%, 55%, 60%, 65%, 70%, 75%, 80%, 85%, 90%, 95%, or 100% identity the corresponding coding tags.

In certain embodiments, an extended recording tag associated with the immobilized polypeptide or peptide may comprise information from multiple coding tags representing multiple, successive binding events. In these embodiments, a single, concatenated extended recording tag associated with the immobilized peptide can be representative of a single polypeptide. As referred to herein, transfer of coding tag information to the recording tag associated with the immobilized peptide also includes transfer to an extended recording tag as would occur in methods involving multiple, successive binding events.

In certain embodiments, the binding event information is transferred from a coding tag to the recording tag associated with the immobilized polypeptide or peptide in a cyclic fashion. Cross-reactive binding events can be informatically filtered out after sequencing by requiring that at least two different coding tags, identifying two or more independent binding events, map to the same class of binding agents (cognate to a particular protein). The coding tag may contain an optional UMI sequence in addition to one or more spacer sequences. Universal priming sequences may also be included in extended nucleic acids on the recording tag associated with the immobilized peptide for amplification and NGS sequencing.

1. Binding Agents

In certain embodiments, the automated methods for the macromolecule, e.g., protein or polypeptide, analysis assay provided in the present disclosure comprise one or more binding cycles, where the polypeptides are contacted with a plurality of binding agents, and successive binding of binding agents transfers historical binding information in the form of a nucleic acid based coding tag to at least one nucleic acid (e.g., recording tag) associated with the polypeptides. In this way, a historical record containing information about multiple binding events is generated in a nucleic acid format.

The methods described herein use a binding agent capable of binding to the macromolecule, e.g., the polypeptide. A binding agent can be any molecule (e.g., peptide, polypeptide, protein, nucleic acid, carbohydrate, small molecule, and the like) capable of binding to a component or feature of a polypeptide. A binding agent can be a naturally occurring, synthetically produced, or recombinantly expressed molecule. In some embodiments, the scaffold used to engineer a binding agent can be from any species, e.g., human, non-human, transgenic. A binding agent may bind to a single monomer or subunit of a polypeptide (e.g., a single amino acid) or bind to multiple linked subunits of a polypeptide (e.g., dipeptide, tripeptide, or higher order peptide of a longer polypeptide molecule).

In certain embodiments, a binding agent may be designed to bind covalently. Covalent binding can be designed to be conditional or favored upon binding to the correct moiety. For example, an NTAA and its cognate NTAA-specific binding agent may each be modified with a reactive group such that once the NTAA-specific binding agent is bound to the cognate NTAA, a coupling reaction is carried out to create a covalent linkage between the two. Non-specific binding of the binding agent to other locations that lack the cognate reactive group would not result in covalent attachment. In some embodiments, the polypeptide comprises a ligand that is capable of forming a covalent bond to a binding agent. In some embodiments, the polypeptide comprises a functionalized NTAA which includes a ligand group that is capable of covalent binding to a binding agent. Covalent binding between a binding agent and its target may allow for more stringent washing to be used to remove binding agents that are non-specifically bound, thus increasing the specificity of the assay.

In certain embodiments, a binding agent may be a selective binding agent. As used herein, selective binding refers to the ability of the binding agent to preferentially bind to a specific ligand (e.g., amino acid or class of amino acids) relative to binding to a different ligand (e.g., amino acid or class of amino acids). Selectivity is commonly referred to as the equilibrium constant for the reaction of displacement of one ligand by another ligand in a complex with a binding agent. Typically, such selectivity is associated with the spatial geometry of the ligand and/or the manner and degree by which the ligand binds to a binding agent, such as by hydrogen bonding, hydrophobic binding, and Van der Waals forces (non-covalent interactions) or by reversible or non-reversible covalent attachment to the binding agent. It should also be understood that selectivity may be relative, and as opposed to absolute, and that different factors can affect the same, including ligand concentration. Thus, in one example, a binding agent selectively binds one of the twenty standard amino acids. In some examples, a binding agent binds to an N-terminal amino acid residue, a C-terminal amino acid residue, or an internal amino acid residue.

In some embodiments, the binding agent is partially specific or selective. In some aspects, the binding agent preferentially binds one or more amino acids. In some examples, a binding agent may bind to or is capable of binding to two or more of the twenty standard amino acids. For example, a binding agent may preferentially bind the amino acids A, C, and G over other amino acids. In some other examples, the binding agent may selectively or specifically bind more than one amino acid. In some aspects, the binding agent may also have a preference for one or more amino acids at the second, third, fourth, fifth, etc. positions from the terminal amino acid. In some cases, the binding agent preferentially binds to a specific terminal amino acid and a penultimate amino acid. For example, a binding agent may preferentially bind AA, AC, and AG or a binding agent may preferentially bind AA, CA, and GA. In some specific examples, binding agents with different specificities can share the same coding tag. In some embodiments, a binding agent may exhibit flexibility and variability in target binding preference in some or all of the positions of the targets. In some examples, a binding agent may have a preference for one or more specific target terminal amino acids and have a flexible preference for a target at the penultimate position. In some other examples, a binding agent may have a preference for one or more specific target amino acids in the penultimate amino acid position and have a flexible preference for a target at the terminal amino acid position. In some embodiments, a binding agent is selective for a target comprising a terminal amino acid and other components of a macromolecule. In some examples, a binding agent is selective for a target comprising a terminal amino acid and at least a portion of the peptide backbone. In some particular examples, a binding agent is selective for a target comprising a terminal amino acid and an amide peptide backbone. In some cases, the peptide backbone comprises a natural peptide backbone or a post-translational modification. In some embodiments, the binding agent exhibits allosteric binding.

In the practice of the methods disclosed herein, the ability of a binding agent to selectively bind to a feature or component of a macromolecule, e.g., a polypeptide, need only be sufficient to allow transfer of its coding tag information to the recording tag associated with the polypeptide. Thus, selectively need only be relative to the other binding agents to which the polypeptide is exposed. It should also be understood that selectivity of a binding agent need not be absolute to a specific amino acid, but could be selective to a class of amino acids, such as amino acids with polar or non-polar side chains, or with electrically (positively or negatively) charged side chains, or with aromatic side chains, or some specific class or size of side chains, and the like. In some embodiments, the ability of a binding agent to selectively bind a feature or component of a macromolecule is characterized by comparing binding abilities of binding agents. For example, the binding ability of a binding agent to the target can be compared to the binding ability of a binding agent which binds to a different target, for example, comparing a binding agent selective for a class of amino acids to a binding agent selective for a different class of amino acids. In some examples, a binding agent selective for non-polar side chains is compared to a binding agent selective for polar side chains. In some embodiments, a binding agent selective for a feature, component of a peptide, or one or more amino acid exhibits at least 1×, at least 2×, at least 5×, at least 10×, at least 50×, at least 100×, or at least 500× more binding compared to a binding agent selective for a different feature, component of a peptide, or one or more amino acid.

In a particular embodiment, the binding agent has a high affinity and high selectivity for the macromolecule, e.g., the polypeptide, of interest. In particular, a high binding affinity with a low off-rate may be efficacious for information transfer between the coding tag and recording tag. In certain embodiments, a binding agent has a Kd of about <500 nM, <200 nM, <100 nM, <50 nM, <10 nM, <5 nM, <1 nM, <0.5 nM, or <0.1 nM. In a particular embodiment, the binding agent is added to the polypeptide at a concentration >1×, >5×, >10×, >100×, or >1000× its Kd to drive binding to completion. For example, binding kinetics of an antibody to a single protein molecule is described in Chang et al., J Immunol Methods (2012) 378(1-2): 102-115.

In certain embodiments, a binding agent may bind to an NTAA, a CTAA, an intervening amino acid, dipeptide (sequence of two amino acids), tripeptide (sequence of three amino acids), or higher order peptide of a peptide molecule. In some embodiments, each binding agent in a library of binding agents selectively binds to a particular amino acid, for example one of the twenty standard naturally occurring amino acids. The standard, naturally-occurring amino acids include Alanine (A or Ala), Cysteine (C or Cys), Aspartic Acid (D or Asp), Glutamic Acid (E or Glu), Phenylalanine (F or Phe), Glycine (G or Gly), Histidine (H or His), Isoleucine (I or Ile), Lysine (K or Lys), Leucine (L or Leu), Methionine (M or Met), Asparagine (N or Asn), Proline (P or Pro), Glutamine (Q or Gln), Arginine (R or Arg), Serine (S or Ser), Threonine (T or Thr), Valine (V or Val), Tryptophan (W or Trp), and Tyrosine (Y or Tyr). In some embodiments, the binding agent binds to an unmodified or native (e.g., natural) amino acid. In some examples, the binding agent binds to an unmodified or native dipeptide (sequence of two amino acids), tripeptide (sequence of three amino acids), or higher order peptide of a peptide molecule. A binding agent may be engineered for high affinity for a native or unmodified NTAA, high specificity for a native or unmodified NTAA, or both. In some embodiments, binding agents can be developed through directed evolution of promising affinity scaffolds using phage display.

In certain embodiments, a binding agent may bind to a post-translational modification of an amino acid. In some embodiments, a peptide comprises one or more post-translational modifications, which may be the same of different. The NTAA, CTAA, an intervening amino acid, or a combination thereof of a peptide may be post-translationally modified. Post-translational modifications to amino acids include acylation, acetylation, alkylation (including methylation), biotinylation, butyrylation, carbamylation, carbonylation, deamidation, deiminiation, diphthamide formation, disulfide bridge formation, eliminylation, flavin attachment, formylation, gamma-carboxylation, glutamylation, glycylation, glycosylation, glypiation, heme C attachment, hydroxylation, hypusine formation, iodination, isoprenylation, lipidation, lipoylation, malonylation, methylation, myristolylation, oxidation, palmitoylation, pegylation, phosphopantetheinylation, phosphorylation, prenylation, propionylation, retinylidene Schiff base formation, S-glutathionylation, S-nitrosylation, S-sulfenylation, selenation, succinylation, sulfination, ubiquitination, and C-terminal amidation (see, also, Seo and Lee, 2004, J. Biochem. Mol. Biol. 37:35-44).

In certain embodiments, a lectin is used as a binding agent for detecting the glycosylation state of a protein, polypeptide, or peptide. Lectins are carbohydrate-binding proteins that can selectively recognize glycan epitopes of free carbohydrates or glycoproteins. A list of lectins recognizing various glycosylation states (e.g., core-fucose, sialic acids, N-acetyl-D-lactosamine, mannose, N-acetyl-glucosamine) include: A, AAA, AAL, ABA, ACA, ACG, ACL, AOL, ASA, BanLec, BC2L-A, BC2LCN, BPA, BPL, Calsepa, CGL2, CNL, Con, ConA, DBA, Discoidin, DSA, ECA, EEL, F17AG, Gal1, Gal1-S, Gal2, Gal3, Gal3C—S, Gal7-S, Gal9, GNA, GRFT, GS-I, GS-II, GSL-I, GSL-II, HHL, HIHA, HPA, I, II, Jacalin, LBA, LCA, LEA, LEL, Lentil, Lotus, LSL-N, LTL, MAA, MAH, MAL_I, Malectin, MOA, MPA, MPL, NPA, Orysata, PA-IIL, PA-IL, PALa, PHA-E, PHA-L, PHA-P, PHAE, PHAL, PNA, PPL, PSA, PSLla, PTL, PTL-I, PWM, RCA120, RS-Fuc, SAMB, SBA, SJA, SNA, SNA-I, SNA-II, SSA, STL, TJA-I, TJA-II, TxLCI, UDA, UEA-I, UEA-II, VFA, VVA, WFA, WGA (see, Zhang et al., 2016, MABS 8:524-535).

In some embodiments, a binding agent may bind to a native or unmodified or unlabeled terminal amino acid. Moreover, in some cases, these natural amino acid binders do not recognize N-terminal labels. Directed evolution of aaRS scaffolds can be used to generate higher affinity, higher specificity binding agents that recognized the N-terminal amino acids in the context of an N-terminal label. In another example, Havranak et al. (U.S. Patent Publication No. US 2014/0273004) describes engineering aminoacyl tRNA synthetases (aaRSs) as specific NTAA binders. The amino acid binding pocket of the aaRSs has an intrinsic ability to bind cognate amino acids, but generally exhibits poor binding affinity and specificity. Moreover, these natural amino acid binders do not recognize N-terminal labels. Directed evolution of aaRS scaffolds can be used to generate higher affinity, higher specificity binding agents that recognized the N-terminal amino acids in the context of an N-terminal label.

In certain embodiments, a binding agent may bind to a modified or labeled terminal amino acid (e.g., an NTAA that has been functionalized or modified). In some embodiments, a binding agent may bind to a chemically or enzymatically modified terminal amino acid. A modified or labeled NTAA can be one that is functionalized with phenylisothiocyanate, PITC, 1-fluoro-2,4-dinitrobenzene (Sanger's reagent, DNFB), benzyloxycarbonyl chloride or carbobenzoxy chloride (Cbz-Cl), N-(Benzyloxycarbonyloxy)succinimide (Cbz-OSu or Cbz-O—NHS), dansyl chloride (DNS-Cl, or 1-dimethylaminonaphthalene-5-sulfonyl chloride), 4-sulfonyl-2-nitrofluorobenzene (SNFB), N-Acetyl-Isatoic Anhydride, Isatoic Anhydride, 2-Pyridinecarboxaldehyde, 2-Formylphenylboronic acid, 2-Acetylphenylboronic acid, 1-Fluoro-2,4-dinitrobenzene, Succinic anhydride, 4-Chloro-7-nitrobenzofurazan, Pentafluorophenylisothiocyanate, 4-(Trifluoromethoxy)-phenylisothiocyanate, 4-(Trifluoromethyl)-phenylisothiocyanate, 3-(Carboxylic acid)-phenylisothiocyanate, 3-(Trifluoromethyl)-phenylisothiocyanate, 1-Naphthylisothiocyanate, N-nitroimidazole-1-carboximidamide, N,N,Ä≤-Bis(pivaloyl)-1H-pyrazole-1-carboxamidine, N,N,Ä≤-Bis(benzyloxycarbonyl)-1H-pyrazole-1-carboxamidine, an acetylating reagent, a guanidinylation reagent, a thioacylation reagent, a thioacetylation reagent, or a thiobenzylation reagent, or a diheterocyclic methanimine reagent. In some examples, the binding agent binds an amino acid labeled by contacting with a reagent or using a method as described in International Patent Publication No. WO 2019/089846. In some cases, the binding agent binds an amino acid labeled by an amine modifying reagent.

In some embodiments, the binding agent binds to a chemically modified N-terminal amino acid residue or a chemically modified C-terminal amino acid residue. To increase the affinity of a binding agent to small N-terminal amino acids (NTAAs) of peptides, the NTAA may be modified with an “immunogenic” hapten, such as dinitrophenol (DNP). This can be implemented in a cyclic sequencing approach using Sanger's reagent, dinitrofluorobenzene (DNFB), which attaches a DNP group to the amine group of the NTAA. Commercial anti-DNP antibodies have affinities in the low nM range (˜8 nM, LO-DNP-2) (Bilgicer et al., J Am Chem Soc (2009) 131(26): 9361-9367); as such it stands to reason that it should be possible to engineer high-affinity NTAA binding agents to a number of NTAAs modified with DNP (via DNFB) and simultaneously achieve good binding selectivity for a particular NTAA. In another example, an NTAA may be modified with sulfonyl nitrophenol (SNP) using 4-sulfonyl-2-nitrofluorobenzene (SNFB). Similar affinity enhancements may also be achieved with alternative NTAA modifiers, such as an acetyl group or an amidinyl (guanidinyl) group.

In certain embodiments, a binding agent can be an aptamer (e.g., peptide aptamer, DNA aptamer, or RNA aptamer), a peptoid, an antibody or a specific binding fragment thereof, an amino acid binding protein or enzyme, an antibody binding fragment, an antibody mimetic, a peptide, a peptidomimetic, a protein, or a polynucleotide (e.g., DNA, RNA, peptide nucleic acid (PNA), a gPNA, bridged nucleic acid (BNA), xeno nucleic acid (XNA), glycerol nucleic acid (GNA), or threose nucleic acid (TNA), or a variant thereof).

As used herein, the terms antibody and antibodies are used in a broad sense, to include not only intact antibody molecules, for example but not limited to immunoglobulin A, immunoglobulin G, immunoglobulin D, immunoglobulin E, and immunoglobulin M, but also any immunoreactive component(s) of an antibody molecule or portion thereof that immuno-specifically bind to at least one epitope. An antibody may be naturally occurring, synthetically produced, or recombinantly expressed. An antibody may be a fusion protein. An antibody may be an antibody mimetic. Examples of antibodies include but are not limited to, Fab fragments, Fab′ fragments, F(ab′)₂ fragments, single chain antibody fragments (scFv), miniantibodies, nanobodies, diabodies, crosslinked antibody fragments, Affibody™, nanobodies, single domain antibodies, DVD-Ig molecules, alphabodies, affimers, affitins, cyclotides, molecules, and the like. Immunoreactive products derived using antibody engineering or protein engineering techniques are also expressly within the meaning of the term antibodies. Detailed descriptions of antibody and/or protein engineering, including relevant protocols, can be found in, among other places, J. Maynard and G. Georgiou, 2000, Ann. Rev. Biomed. Eng. 2:339-76; Antibody Engineering, R. Kontermann and S. Dubel, eds., Springer Lab Manual, Springer Verlag (2001); U.S. Pat. No. 5,831,012; and S. Paul, Antibody Engineering Protocols, Humana Press (1995).

As with antibodies, nucleic acid and peptide aptamers that specifically recognize a macromolecule, e.g., a peptide or a polypeptide, can be produced using known methods. Aptamers bind target molecules in a highly specific, conformation-dependent manner, typically with very high affinity, although aptamers with lower binding affinity can be selected if desired. Aptamers have been shown to distinguish between targets based on very small structural differences such as the presence or absence of a methyl or hydroxyl group and certain aptamers can distinguish between D- and L-enantiomers. Aptamers have been obtained that bind small molecular targets, including drugs, metal ions, and organic dyes, peptides, biotin, and proteins, including but not limited to streptavidin, VEGF, and viral proteins. Aptamers have been shown to retain functional activity after biotinylation, fluorescein labeling, and when attached to glass surfaces and microspheres. (see, e.g., Jayasena, 1999, Clin Chem 45:1628-50; Kusser 2000, J. Biotechnol. 74: 27-39; Colas, 2000, Curr Opin Chem Biol 4:54-9). Aptamers which specifically bind arginine and AMP have been described as well (see, Patel and Suri, 2000, J. Biotech. 74:39-60). Oligonucleotide aptamers that bind to a specific amino acid have been disclosed in Gold et al. (1995, Ann. Rev. Biochem. 64:763-97). RNA aptamers that bind amino acids have also been described (Ames and Breaker, 2011, RNA Biol. 8; 82-89; Mannironi et al., 2000, RNA 6:520-27; Famulok, 1994, J. Am. Chem. Soc. 116:1698-1706).

A binding agent can be made by modifying naturally-occurring or synthetically-produced proteins by genetic engineering to introduce one or more mutations in the amino acid sequence to produce engineered proteins that bind to a specific component or feature of a polypeptide (e.g., NTAA, CTAA, or post-translationally modified amino acid or a peptide). For example, exopeptidases (e.g., aminopeptidases, carboxypeptidases), exoproteases, mutated exoproteases, mutated anticalins, mutated ClpSs, antibodies, or tRNA synthetases can be modified to create a binding agent that selectively binds to a particular NTAA. In another example, carboxypeptidases can be modified to create a binding agent that selectively binds to a particular CTAA. A binding agent can also be designed or modified, and utilized, to specifically bind a modified NTAA or modified CTAA, for example one that has a post-translational modification (e.g., phosphorylated NTAA or phosphorylated CTAA) or one that has been modified with a label (e.g., PTC, 1-fluoro-2,4-dinitrobenzene (using Sanger's reagent, DNFB), dansyl chloride (using DNS-Cl, or 1-dimethylaminonaphthalene-5-sulfonyl chloride), or using a thioacylation reagent, a thioacetylation reagent, an acetylation reagent, an amidination (guanidinylation) reagent, or a thiobenzylation reagent). Strategies for directed evolution of proteins are known in the art (e.g., Yuan et al., 2005, Microbiol. Mol. Biol. Rev. 69:373-392), and include phage display, ribosomal display, mRNA display, CIS display, CAD display, emulsions, cell surface display method, yeast surface display, bacterial surface display, etc.

In some embodiments, a binding agent that selectively binds to a labeled or functionalized NTAA can be utilized. For example, the NTAA may be reacted with phenylisothiocyanate (PITC) to form a phenylthiocarbamoyl-NTAA derivative. In this manner, the binding agent may be fashioned to selectively bind both the phenyl group of the phenylthiocarbamoyl moiety as well as the alpha-carbon R group of the NTAA. Use of PITC in this manner allows for subsequent elimination of the NTAA by Edman degradation as discussed below. In another embodiment, the NTAA may be reacted with Sanger's reagent (DNFB), to generate a DNP-labeled NTAA. Optionally, DNFB is used with an ionic liquid such as 1-ethyl-3-methylimidazolium bis[(trifluoromethyl)sulfonyl]imide ([emim][Tf2N]), in which DNFB is highly soluble. In this manner, the binding agent may be engineered to selectively bind the combination of the DNP and the R group on the NTAA. The addition of the DNP moiety provides a larger “handle” for the interaction of the binding agent with the NTAA, and should lead to a higher affinity interaction.

In yet another embodiment, a binding agent may be a modified aminopeptidase. In some embodiments, the binding agent may be a modified aminopeptidase that has been engineered to recognize the DNP-labeled NTAA providing cyclic control of aminopeptidase degradation of the peptide. Once the DNP-labeled NTAA is eliminated, another cycle of DNFB derivatization is performed in order to bind and eliminate the newly exposed NTAA. In preferred particular embodiment, the aminopeptidase is a monomeric metallo-protease, such an aminopeptidase activated by zinc (Calcagno et al., Appl Microbiol Biotechnol. (2016) 100(16):7091-7102). In another example, a binding agent may selectively bind to an NTAA that is modified with sulfonyl nitrophenol (SNP), e.g., by using 4-sulfonyl-2-nitrofluorobenzene (SNFB). Other reagents that may be used to functionalize the NTAA include trifluoroethyl isothiocyanate, allyl isothiocyanate, and dimethylaminoazobenzene isothiocyanate, or a reagent as described in International Patent Publication No. WO 2019/089846.

A binding agent may be engineered for high affinity for a modified NTAA, high specificity for a modified NTAA, or both. In some embodiments, binding agents can be developed through directed evolution of promising affinity scaffolds using phage display.

In another example, highly-selective engineered ClpSs have also been described in the literature. Emili et al. describe the directed evolution of an E. coli ClpS protein via phage display, resulting in four different variants with the ability to selectively bind NTAAs for aspartic acid, arginine, tryptophan, and leucine residues (U.S. Pat. No. 9,566,335, incorporated by reference in its entirety). In one embodiment, the binding moiety of the binding agent comprises a member of the evolutionarily conserved ClpS family of adaptor proteins involved in natural N-terminal protein recognition and binding or a variant thereof. (See e.g., Schuenemann et al., (2009) EMBO Reports 10(5); Roman-Hernandez et al., (2009) PNAS 106(22):8888-93; Guo et al., (2002) JBC 277(48): 46753-62; Wang et al., (2008) Molecular Cell 32: 406-414). In some embodiments, the amino acid residues corresponding to the ClpS hydrophobic binding pocket identified in Schuenemann et al. are modified in order to generate a binding moiety with the desired selectivity.

In one embodiment, the binding moiety comprises a member of the UBR box recognition sequence family, or a variant of the UBR box recognition sequence family. UBR recognition boxes are described in Tasaki et al., (2009), JBC 284(3): 1884-95. For example, the binding moiety may comprise UBR1, UBR2, or a mutant, variant, or homologue thereof.

In certain embodiments, the binding agent further comprises one or more detectable labels such as fluorescent labels, in addition to the binding moiety. In some embodiments, the binding agent does not comprise a polynucleotide such as a coding tag. Optionally, the binding agent comprises a synthetic or natural antibody. In some embodiments, the binding agent comprises an aptamer. In one embodiment, the binding agent comprises a polypeptide, such as a modified member of the ClpS family of adaptor proteins, such as a variant of an E. coli ClpS binding polypeptide, and a detectable label. In one embodiment, the detectable label is optically detectable. In some embodiments, the detectable label comprises a fluorescently moiety, a color-coded nanoparticle, a quantum dot or any combination thereof. In one embodiment the label comprises a polystyrene dye encompassing a core dye molecule such as a FluoSphere™, Nile Red, fluorescein, rhodamine, derivatized rhodamine dyes, such as TAMRA, phosphor, polymethadine dye, fluorescent phosphoramidite, TEXAS RED, green fluorescent protein, acridine, cyanine, cyanine 5 dye, cyanine 3 dye, 5-(2′-aminoethyl)-aminonaphthalene-1-sulfonic acid (EDANS), BODIPY, 120 ALEXA or a derivative or modification of any of the foregoing. In one embodiment, the detectable label is resistant to photobleaching while producing lots of signal (such as photons) at a unique and easily detectable wavelength, with high signal-to-noise ratio.

In a particular embodiment, anticalins are engineered for both high affinity and high specificity to labeled NTAAs (e.g. PTC, modified-PTC, Cbz, DNP, SNP, acetyl, guanidinyl, amino guanidinyl, heterocyclic methanimine, etc.). Certain varieties of anticalin scaffolds have suitable shape for binding single amino acids, by virtue of their beta barrel structure. An N-terminal amino acid (either with or without modification) can potentially fit and be recognized in this “beta barrel” bucket. High affinity anticalins with engineered novel binding activities have been described (reviewed by Skerra, 2008, FEBS J. 275: 2677-2683). For example, anticalins with high affinity binding (low nM) to fluorescein and digoxygenin have been engineered (Gebauer et al., 2012, Methods Enzymol 503: 157-188.). Engineering of alternative scaffolds for new binding functions has also been reviewed by Banta et al. (2013, Annu. Rev. Biomed. Eng. 15:93-113).

The functional affinity (avidity) of a given monovalent binding agent may be increased by at least an order of magnitude by using a bivalent or higher order multimer of the monovalent binding agent (Vauquelin et al., 2013, Br J Pharmacol 168(8): 1771-1785. 2013). Avidity refers to the accumulated strength of multiple, simultaneous, non-covalent binding interactions. An individual binding interaction may be easily dissociated. However, when multiple binding interactions are present at the same time, transient dissociation of a single binding interaction does not allow the binding protein to diffuse away and the binding interaction is likely to be restored. An alternative method for increasing avidity of a binding agent is to include complementary sequences in the coding tag attached to the binding agent and the recording tag associated with the polypeptide.

In some embodiments, the binding agent is derived from a biological, naturally occurring, non-naturally occurring, or synthetic source. In some examples, the binding agent is derived from de novo protein design (Huang et al., (2016) 537(7620):320-327). In some examples, the binding agent has a structure, sequence, and/or activity designed from first principles.

In some embodiments, a binding agent can be utilized that selectively binds a modified C-terminal amino acid (CTAA). Carboxypeptidases are proteases that cleave/eliminate terminal amino acids containing a free carboxyl group. A number of carboxypeptidases exhibit amino acid preferences, e.g., carboxypeptidase B preferentially cleaves at basic amino acids, such as arginine and lysine. A carboxypeptidase can be modified to create a binding agent that selectively binds to particular amino acid. In some embodiments, the carboxypeptidase may be engineered to selectively bind both the modification moiety as well as the alpha-carbon R group of the CTAA. Thus, engineered carboxypeptidases may specifically recognize 20 different CTAAs representing the standard amino acids in the context of a C-terminal label. Control of the stepwise degradation from the C-terminus of the peptide is achieved by using engineered carboxypeptidases that are only active (e.g., binding activity or catalytic activity) in the presence of the label. In one example, the CTAA may be modified by a para-Nitroanilide or 7-amino-4-methylcoumarinyl group.

Other potential scaffolds that can be engineered to generate binding agents for use in the methods described herein include: an anticalin, a lipocalin, an amino acid tRNA synthetase (aaRS), ClpS, an Affilin-, an Adnectin™, a T cell receptor, a zinc finger protein, a thioredoxin, GST A1-1, DARPin, an affimer, an affitin, an alphabody, an avimer, a monobody, an antibody, a single domain antibody, a nanobody, EETI-II, HPSTI, intrabody, PHD-finger, V(NAR) LDTI, evibody, Ig(NAR), knottin, maxibody, microbody, neocarzinostatin, pVIII, tendamistat, VLR, protein A scaffold, MTI-II, ecotin, GCN4, Im9, kunitz domain, PBP, trans-body, tetranectin, WW domain, CBM4-2, DX-88, GFP, iMab, Ldl receptor domain A, Min-23, PDZ-domain, avian pancreatic polypeptide, charybdotoxin/10Fn3, domain antibody (Dab), a2p8 ankyrin repeat, insect defensing A peptide, Designed AR protein, C-type lectin domain, staphylococcal nuclease, Src homology domain 3 (SH3), or Src homology domain 2 (SH2). See e.g., El-Gebali et al., (2019) Nucleic Acids Research 47:D427-D432 and Finn et al., (2013) Nucleic Acids Res. 42 (Database issue):D222-D230. In some embodiments, a binding agent is derived from an enzyme which binds one or more amino acids (e.g., an aminopeptidase). In certain embodiments, a binding agent can be derived from an anticalin or a Clp protease adaptor protein (ClpS).

A binding agent may preferably bind to a modified or labeled amino acid, by chemical or enzymatic means, (e.g., an amino acid that has been functionalized by a reagent (e.g., a compound)) over a non-modified or unlabeled amino acid. For example, a binding agent may preferably bind to an amino acid that has been functionalized with an acetyl moiety, Cbz moiety, guanyl moiety, dansyl moiety, PTC moiety, DNP moiety, SNP moiety, diheterocyclic methanimine moiety, etc., over an amino acid that does not possess said moiety. In some embodiments, a binding agent may preferably bind to an amino acid that has been functionalized or modified as described in International Patent Publication No. WO 2019/089846. In some cases, a binding agent may bind to a post-translationally modified amino acid. Thus, in certain embodiments, an extended nucleic acid associated with the comprises coding tag information relating to amino acid sequence and post-translational modifications of the polypeptide. In some embodiments, detection of internal post-translationally modified amino acids (e.g., phosphorylation, glycosylation, succinylation, ubiquitination, S-Nitrosylation, methylation, N-acetylation, lipidation, etc.) is be accomplished prior to detection and elimination of terminal amino acids (e.g., NTAA or CTAA). In one example, a peptide is contacted with binding agents for PTM modifications, and associated coding tag information are transferred to the recording tag associated with the immobilized peptide. Once the detection and transfer of coding tag information relating to amino acid modifications is complete, the PTM modifying groups can be removed before detection and transfer of coding tag information for the primary amino acid sequence using N-terminal or C-terminal degradation methods. Thus, resulting extended nucleic acids indicate the presence of post-translational modifications in a peptide sequence, though not the sequential order, along with primary amino acid sequence information.

In some embodiments, detection of internal post-translationally modified amino acids may occur concurrently with detection of primary amino acid sequence. In one example, an NTAA (or CTAA) is contacted with a binding agent specific for a post-translationally modified amino acid, either alone or as part of a library of binding agents (e.g., library composed of binding agents for the 20 standard amino acids and selected post-translational modified amino acids). Successive cycles of terminal amino acid elimination and contact with a binding agent (or library of binding agents) follow. Thus, resulting extended nucleic acids on the recording tag associated with the immobilized peptide indicate the presence and order of post-translational modifications in the context of a primary amino acid sequence.

In certain embodiments, a macromolecule, e.g., a polypeptide, is also contacted with a non-cognate binding agent. As used herein, a non-cognate binding agent is referring to a binding agent that is selective for a different polypeptide feature or component than the particular polypeptide being considered. For example, if the n NTAA is phenylalanine, and the peptide is contacted with three binding agents selective for phenylalanine, tyrosine, and asparagine, respectively, the binding agent selective for phenylalanine would be first binding agent capable of selectively binding to the n-NTAA (i.e., phenylalanine), while the other two binding agents would be non-cognate binding agents for that peptide (since they are selective for NTAAs other than phenylalanine). The tyrosine and asparagine binding agents may, however, be cognate binding agents for other peptides in the sample. If the n NTAA (phenylalanine) was then cleaved from the peptide, thereby converting the n-1 amino acid of the peptide to the n-1 NTAA (e.g., tyrosine), and the peptide was then contacted with the same three binding agents, the binding agent selective for tyrosine would be second binding agent capable of selectively binding to the n-1 NTAA (i.e., tyrosine), while the other two binding agents would be non-cognate binding agents (since they are selective for NTAAs other than tyrosine).

Thus, it should be understood that whether an agent is a binding agent or a non-cognate binding agent will depend on the nature of the particular polypeptide feature or component currently available for binding. Also, if multiple polypeptides are analyzed in a multiplexed reaction, a binding agent for one polypeptide may be a non-cognate binding agent for another, and vice versa. According, it should be understood that the following description concerning binding agents is applicable to any type of binding agent described herein (i.e., both cognate and non-cognate binding agents).

Any binding agent described comprises a coding tag containing identifying information regarding the binding agent. A coding tag is a nucleic acid molecule of about 3 bases to about 100 bases that provides unique identifying information for its associated binding agent. A coding tag may comprise about 3 to about 90 bases, about 3 to about 80 bases, about 3 to about 70 bases, about 3 to about 60 bases, about 3 bases to about 50 bases, about 3 bases to about 40 bases, about 3 bases to about 30 bases, about 3 bases to about 20 bases, about 3 bases to about 10 bases, or about 3 bases to about 8 bases. In some embodiments, a coding tag is about 3 bases, 4 bases, 5 bases, 6 bases, 7 bases, 8 bases, 9 bases, 10 bases, 11 bases, 12 bases, 13 bases, 14 bases, 15 bases, 16 bases, 17 bases, 18 bases, 19 bases, 20 bases, 25 bases, 30 bases, 35 bases, 40 bases, 55 bases, 60 bases, 65 bases, 70 bases, 75 bases, 80 bases, 85 bases, 90 bases, 95 bases, or 100 bases in length. A coding tag may be composed of DNA, RNA, polynucleotide analogs, or a combination thereof. Polynucleotide analogs include PNA, gPNA, BNA, GNA, TNA, LNA, morpholino polynucleotides, 2′-O-Methyl polynucleotides, alkyl ribosyl substituted polynucleotides, phosphorothioate polynucleotides, and 7-deaza purine analogs.

A coding tag comprises an encoder sequence that provides identifying information regarding the associated binding agent. An encoder sequence is about 3 bases to about 30 bases, about 3 bases to about 20 bases, about 3 bases to about 10 bases, or about 3 bases to about 8 bases. In some embodiments, an encoder sequence is about 3 bases, 4 bases, 5 bases, 6 bases, 7 bases, 8 bases, 9 bases, 10 bases, 11 bases, 12 bases, 13 bases, 14 bases, 15 bases, 20 bases, 25 bases, or 30 bases in length. The length of the encoder sequence determines the number of unique encoder sequences that can be generated. Shorter encoding sequences generate a smaller number of unique encoding sequences, which may be useful when using a small number of binding agents. In a specific embodiment, a set of >50 unique encoder sequences are used for a binding agent library.

In some embodiments, each unique binding agent within a library of binding agents has a unique encoder sequence. For example, 20 unique encoder sequences may be used for a library of 20 binding agents that bind to the 20 standard amino acids. Additional coding tag sequences may be used to identify modified amino acids (e.g., post-translationally modified amino acids). In another example, 30 unique encoder sequences may be used for a library of 30 binding agents that bind to the 20 standard amino acids and 10 post-translational modified amino acids (e.g., phosphorylated amino acids, acetylated amino acids, methylated amino acids). In other embodiments, two or more different binding agents may share the same encoder sequence. For example, two binding agents that each bind to a different standard amino acid may share the same encoder sequence.

In certain embodiments, a coding tag further comprises a spacer sequence at one end or both ends. A spacer sequence is about 1 base to about 20 bases, about 1 base to about 10 bases, about 5 bases to about 9 bases, or about 4 bases to about 8 bases. In some embodiments, a spacer is about 1 base, 2 bases, 3 bases, 4 bases, 5 bases, 6 bases, 7 bases, 8 bases, 9 bases, 10 bases, 11 bases, 12 bases, 13 bases, 14 bases, 15 bases or 20 bases in length. In some embodiments, a spacer within a coding tag is shorter than the encoder sequence, e.g., at least 1 base, 2, bases, 3 bases, 4 bases, 5 bases, 6, bases, 7 bases, 8 bases, 9 bases, 10 bases, 11 bases, 12 bases, 13 bases, 14 bases, 15 bases, 20 bases, or 25 bases shorter than the encoder sequence. In other embodiments, a spacer within a coding tag is the same length as the encoder sequence. In certain embodiments, the spacer is binding agent specific so that a spacer from a previous binding cycle only interacts with a spacer from the appropriate binding agent in a current binding cycle. An example would be pairs of cognate antibodies containing spacer sequences that only allow information transfer if both antibodies sequentially bind to the polypeptide. A spacer sequence may be used as the primer annealing site for a primer extension reaction, or a splint or sticky end in a ligation reaction. A 5′ spacer on a coding tag may optionally contain pseudo complementary bases to a 3′ spacer on the recording tag to increase T. (Lehoud et al., 2008, Nucleic Acids Res. 36:3409-3419). In other embodiments, the coding tags within a library of binding agents do not have a binding cycle specific spacer sequence.

In one example, two or more binding agents that each bind to different targets have associated coding tags share the same spacers. In some cases, coding tags associated with two or more binding agents share coding tags with the same sequence or a portion thereof.

In some embodiments, the coding tags within a collection of binding agents share a common spacer sequence used in an assay (e.g. the entire library of binding agents used in a multiple binding cycle method possess a common spacer in their coding tags). In another embodiment, the coding tags are comprised of a binding cycle tags, identifying a particular binding cycle. In other embodiments, the coding tags within a library of binding agents have a binding cycle specific spacer sequence. In some embodiments, a coding tag comprises one binding cycle specific spacer sequence. For example, a coding tag for binding agents used in the first binding cycle comprise a “cycle 1” specific spacer sequence, a coding tag for binding agents used in the second binding cycle comprise a “cycle 2” specific spacer sequence, and so on up to “n” binding cycles. In further embodiments, coding tags for binding agents used in the first binding cycle comprise a “cycle 1” specific spacer sequence and a “cycle 2” specific spacer sequence, coding tags for binding agents used in the second binding cycle comprise a “cycle 2” specific spacer sequence and a “cycle 3” specific spacer sequence, and so on up to “n” binding cycles. In some embodiments, a spacer sequence comprises a sufficient number of bases to anneal to a complementary spacer sequence in a recording tag or extended recording tag to initiate a primer extension reaction or sticky end ligation reaction.

In some embodiments, coding tags associated with binding agents used to bind in an alternating cycles comprises different binding cycle specific spacer sequences. For example, a coding tag for binding agents used in the first binding cycle comprise a “cycle 1” specific spacer sequence, a coding tag for binding agents used in the second binding cycle comprise a “cycle 2” specific spacer sequence, a coding tag for binding agents used in the third binding cycle also comprises the “cycle 1” specific spacer sequence, a coding tag for binding agents used in the fourth binding cycle comprises the “cycle 2” specific spacer sequence. In this manner, cycle specific spacers are not needed for every cycle.

A cycle specific spacer sequence can also be used to concatenate information of coding tags onto a single recording tag when a population of recording tags is associated with a polypeptide. The first binding cycle transfers information from the coding tag to a randomly-chosen recording tag, and subsequent binding cycles can prime only the extended recording tag using cycle dependent spacer sequences. More specifically, coding tags for binding agents used in the first binding cycle comprise a “cycle 1” specific spacer sequence and a “cycle 2” specific spacer sequence, coding tags for binding agents used in the second binding cycle comprise a “cycle 2” specific spacer sequence and a “cycle 3” specific spacer sequence, and so on up to “n” binding cycles. Coding tags of binding agents from the first binding cycle are capable of annealing to recording tags via complementary cycle 1 specific spacer sequences. Upon transfer of the coding tag information to the recording tag, the cycle 2 specific spacer sequence is positioned at the 3′ terminus of the extended recording tag at the end of binding cycle 1. Coding tags of binding agents from the second binding cycle are capable of annealing to the extended recording tags via complementary cycle 2 specific spacer sequences. Upon transfer of the coding tag information to the extended recording tag, the cycle 3 specific spacer sequence is positioned at the 3′ terminus of the extended recording tag at the end of binding cycle 2, and so on through “n” binding cycles. This embodiment provides that transfer of binding information in a particular binding cycle among multiple binding cycles will only occur on (extended) recording tags that have experienced the previous binding cycles. However, sometimes a binding agent may fail to bind to a cognate polypeptide. Oligonucleotides comprising binding cycle specific spacers after each binding cycle as a “chase” step can be used to keep the binding cycles synchronized even if the event of a binding cycle failure. For example, if a cognate binding agent fails to bind to a polypeptide during binding cycle 1, adding a chase step following binding cycle 1 using oligonucleotides comprising both a cycle 1 specific spacer, a cycle 2 specific spacer, and a “null” encoder sequence. The “null” encoder sequence can be the absence of an encoder sequence or, preferably, a specific barcode that positively identifies a “null” binding cycle. The “null” oligonucleotide is capable of annealing to the recording tag via the cycle 1 specific spacer, and the cycle 2 specific spacer is transferred to the recording tag. Thus, binding agents from binding cycle 2 are capable of annealing to the extended recording tag via the cycle 2 specific spacer despite the failed binding cycle 1 event. The “null” oligonucleotide marks binding cycle 1 as a failed binding event within the extended recording tag.

In one embodiment, binding cycle-specific encoder sequences are used in coding tags. Binding cycle-specific encoder sequences may be accomplished either via the use of completely unique analyte (e.g., NTAA)-binding cycle encoder barcodes or through a combinatoric use of an analyte (e.g., NTAA) encoder sequence joined to a cycle-specific barcode. The advantage of using a combinatoric approach is that fewer total barcodes need to be designed. For a set of 20 analyte binding agents used across 10 cycles, only 20 analyte encoder sequence barcodes and 10 binding cycle specific barcodes need to be designed. In contrast, if the binding cycle is embedded directly in the binding agent encoder sequence, then a total of 200 independent encoder barcodes may need to be designed. An advantage of embedding binding cycle information directly in the encoder sequence is that the total length of the coding tag can be minimized when employing error-correcting barcodes. The use of error-tolerant barcodes allows highly accurate barcode identification using sequencing platforms and approaches that are more error-prone, but have other advantages such as rapid speed of analysis, lower cost, and/or more portable instrumentation.

In some embodiments, a coding tag comprises a cleavable or nickable DNA strand within the second (3′) spacer sequence proximal to the binding agent. For example, the 3′ spacer may have one or more uracil bases that can be nicked by uracil-specific excision reagent (USER). USER generates a single nucleotide gap at the location of the uracil. In another example, the 3′ spacer may comprise a recognition sequence for a nicking endonuclease that hydrolyzes only one strand of a duplex. Preferably, the enzyme used for cleaving or nicking the 3′ spacer sequence acts only on one DNA strand (the 3′ spacer of the coding tag), such that the other strand within the duplex belonging to the (extended) recording tag is left intact. These embodiments is particularly useful in assays analyzing proteins in their native conformation, as it allows the non-denaturing removal of the binding agent from the (extended) recording tag after primer extension has occurred and leaves a single stranded DNA spacer sequence on the extended recording tag available for subsequent binding cycles.

The coding tags may also be designed to contain palindromic sequences. Inclusion of a palindromic sequence into a coding tag allows a nascent, growing, extended recording tag to fold upon itself as coding tag information is transferred. The extended recording tag is folded into a more compact structure, effectively decreasing undesired inter-molecular binding and primer extension events.

An extended recording tag can be built up from a series of binding events using coding tags comprising analyte-specific spacers and encoder sequences. In one embodiment, a first binding event employs a binding agent with a coding tag comprised of a generic 3′ spacer primer sequence and an analyte-specific spacer sequence at the 5′ terminus for use in the next binding cycle; subsequent binding cycles then use binding agents with encoded analyte-specific 3′ spacer sequences. This design results in amplifiable library elements being created only from a correct series of cognate binding events. Off-target and cross-reactive binding interactions will lead to a non-amplifiable extended recording tag. In one example, a pair of cognate binding agents to a particular polypeptide analyte is used in two binding cycles to identify the analyte. The first cognate binding agent contains a coding tag comprised of a generic spacer 3′ sequence for priming extension on the generic spacer sequence of the recording tag, and an encoded analyte-specific spacer at the 5′ end, which will be used in the next binding cycle. For matched cognate binding agent pairs, the 3′ analyte-specific spacer of the second binding agent is matched to the 5′ analyte-specific spacer of the first binding agent. In this way, only correct binding of the cognate pair of binding agents will result in an amplifiable extended recording tag. Cross-reactive binding agents will not be able to prime extension on the recording tag, and no amplifiable extended recording tag product generated. This approach greatly enhances the specificity of the methods disclosed herein. The same principle can be applied to triplet binding agent sets, in which 3 cycles of binding are employed. In a first binding cycle, a generic 3′ Sp sequence on the recording tag interacts with a generic spacer on a binding agent coding tag. Primer extension transfers coding tag information, including an analyte specific 5′ spacer, to the recording tag. Subsequent binding cycles employ analyte specific spacers on the binding agents' coding tags.

In certain embodiments, a coding tag may further comprise a unique molecular identifier for the binding agent to which the coding tag is linked.

A coding tag may include a terminator nucleotide incorporated at the 3′ end of the 3′ spacer sequence. After a binding agent binds to a polypeptide and their corresponding coding tag and recording tags anneal via complementary spacer sequences, it is possible for primer extension to transfer information from the coding tag to the recording tag, or to transfer information from the recording tag to the coding tag. Addition of a terminator nucleotide on the 3′ end of the coding tag prevents transfer of recording tag information to the coding tag. It is understood that for embodiments described herein involving generation of extended coding tags, it may be preferable to include a terminator nucleotide at the 3′ end of the recording tag to prevent transfer of coding tag information to the recording tag.

A coding tag may be a single stranded molecule, a double stranded molecule, or a partially double stranded. A coding tag may comprise blunt ends, overhanging ends, or one of each. In some embodiments, a coding tag is partially double stranded, which prevents annealing of the coding tag to internal encoder and spacer sequences in a growing extended recording tag. In some embodiments, the coding tag comprises a hairpin. In certain embodiments, the hairpin comprises mutually complementary nucleic acid regions are connected through a nucleic acid strand. In some embodiments, the nucleic acid hairpin can also further comprise 3′ and/or 5′ single-stranded region(s) extending from the double-stranded stem segment. In some examples, the hairpin comprises a single strand of nucleic acid.

In some embodiments, the coding tag sequence can be optimized for the particular sequencing analysis platform. In a particular embodiment, the sequencing platform is nanopore sequencing. In some embodiments, the sequencing platform has a per base error rate of >1%, >5%, >10%, >15%, >20%, >25%, or >30%. For example, if the extended nucleic acid is to be analyzed using a nanopore sequencing instrument, the barcode sequences (e.g., sequences comprising identifying information from the coding tag) can be designed to be optimally electrically distinguishable in transit through a nanopore.

In some embodiments, a coding tag may include a terminator nucleotide incorporated at the 3′ end of the 3′ spacer sequence. After a binding agent binds to a macromolecule and their corresponding coding tag and recording tags anneal via complementary spacer sequences, it is possible for primer extension to transfer information from the coding tag to the recording tag, or to transfer information from the recording tag to the coding tag. Addition of a terminator nucleotide on the 3′ end of the coding tag prevents transfer of recording tag information to the coding tag. It is understood that for embodiments described herein involving generation of extended coding tags, it may be preferable to include a terminator nucleotide at the 3′ end of the recording tag to prevent transfer of coding tag information to the recording tag.

A coding tag can be joined to a binding agent directly or indirectly, by any means known in the art, including covalent and non-covalent interactions. In some embodiments, a coding tag may be joined to binding agent enzymatically or chemically. In some embodiments, a coding tag may be joined to a binding agent via ligation. In other embodiments, a coding tag is joined to a binding agent via affinity binding pairs (e.g., biotin and streptavidin). In some cases, a coding tag may be joined to a binding agent to an unnatural amino acid, such as via a covalent interaction with an unnatural amino acid.

In some embodiments, a binding agent is joined to a coding tag via SpyCatcher-SpyTag interaction. The SpyTag peptide forms an irreversible covalent bond to the SpyCatcher protein via a spontaneous isopeptide linkage, thereby offering a genetically encoded way to create peptide interactions that resist force and harsh conditions (Zakeri et al., 2012, Proc. Natl. Acad. Sci. 109:E690-697; Li et al., 2014, J. Mol. Biol. 426:309-317). A binding agent may be expressed as a fusion protein comprising the SpyCatcher protein. In some embodiments, the SpyCatcher protein is appended on the N-terminus or C-terminus of the binding agent. The SpyTag peptide can be coupled to the coding tag using standard conjugation chemistries (Bioconjugate Techniques, G. T. Hermanson, Academic Press (2013)).

In some embodiments, an enzyme-based strategy is used to join the binding agent to a coding tag. For example, the binding agent may be joined to a coding tag using a formylglycine (FGly)-generating enzyme (FGE). In one example, a protein, e.g., SpyLigase, is used to join the binding agent to the coding tag (Fierer et al., Proc Natl Acad Sci USA. 2014; 111 (13): E1176-E1181).

In other embodiments, a binding agent is joined to a coding tag via SnoopTag-SnoopCatcher peptide-protein interaction. The SnoopTag peptide forms an isopeptide bond with the SnoopCatcher protein (Veggiani et al., Proc. Natl. Acad. Sci. USA, 2016, 113:1202-1207). A binding agent may be expressed as a fusion protein comprising the SnoopCatcher protein. In some embodiments, the SnoopCatcher protein is appended on the N-terminus or C-terminus of the binding agent. The SnoopTag peptide can be coupled to the coding tag using standard conjugation chemistries.

In yet other embodiments, a binding agent is joined to a coding tag via the HaloTag® protein fusion tag and its chemical ligand. HaloTag is a modified haloalkane dehalogenase designed to covalently bind to synthetic ligands (HaloTag ligands) (Los et al., 2008, ACS Chem. Biol. 3:373-382). The synthetic ligands comprise a chloroalkane linker attached to a variety of useful molecules. A covalent bond forms between the HaloTag and the chloroalkane linker that is highly specific, occurs rapidly under physiological conditions, and is essentially irreversible.

In some cases, a binding agent is joined to a coding tag by attaching (conjugating) using an enzyme, such as sortase-mediated labeling (See e.g., Antos et al., Curr Protoc Protein Sci. (2009) CHAPTER 15: Unit-15.3; International Patent Publication No. WO2013003555). The sortase enzyme catalyzes a transpeptidation reaction (See e.g., Falck et al, Antibodies (2018) 7(4):1-19). In some aspects, the binding agent is modified with or attached to one or more N-terminal or C-terminal glycine residues.

In some embodiments, a binding agent is joined to a coding tag using a cysteine bioconjugation method. In some embodiments, a binding agent is joined to a coding tag using 7r-clamp-mediated cysteine bioconjugation (See e.g., Zhang et al., Nat Chem. (2016) 8(2):120-128). In some cases, a binding agent is joined to a coding tag using 3-arylpropiolonitriles (APN)-mediated tagging (e.g. Koniev et al., Bioconjug Chem. 2014; 25(2):202-206).

In some embodiments, the binding agent is linked, directly or indirectly, to a multimerization domain. Thus, monomeric, dimeric, and higher order (e.g., 3, 4, 5, or more) multimeric polypeptides comprising one or more binding agents are provided herein. In some specific embodiments, the binding agent is dimeric. In some examples, two polypeptides of the invention can be covalently or non-covalently attached to each other to form a dimer.

In some embodiments, contacting of the first binding agent and second binding agent to the polypeptide, and optionally any further binding agents (e.g., third binding agent, fourth binding agent, fifth binding agent, and so on), are performed at the same time. For example, the first binding agent and second binding agent, and optionally any further order binding agents, can be pooled together, for example to form a library of binding agents. In another example, the first binding agent and second binding agent, and optionally any further order binding agents, rather than being pooled together, are added simultaneously to the polypeptide. In one embodiment, a library of binding agents comprises at least 20 binding agents that selectively bind to the 20 standard, naturally occurring amino acids. In some embodiments, a library of binding agents may comprise binding agents that selectively bind to the modified amino acids.

In other embodiments, the first binding agent and second binding agent, and optionally any further order binding agents, are each contacted with the polypeptide in separate binding cycles, added in sequential order. In certain embodiments, multiple binding agents are used at the same time in parallel. This parallel approach saves time and reduces non-specific binding by non-cognate binding agents to a site that is bound by a cognate binding agent (because the binding agents are in competition).

In certain embodiments, the concentration of the binding agents in a solution is controlled to reduce background and/or false positive results of the assay.

In some embodiments, the concentration of a binding agent can be at any suitable concentration, e.g., at about 0.0001 nM, about 0.001 nM, about 0.01 nM, about 0.1 nM, about 1 nM, about 2 nM, about 5 nM, about 10 nM, about 20 nM, about 50 nM, about 100 nM, about 200 nM, about 500 nM, or about 1,000 nM. In other embodiments, the concentration of a soluble conjugate used in the assay is between about 0.0001 nM and about 0.001 nM, between about 0.001 nM and about 0.01 nM, between about 0.01 nM and about 0.1 nM, between about 0.1 nM and about 1 nM, between about 1 nM and about 2 nM, between about 2 nM and about 5 nM, between about 5 nM and about 10 nM, between about 10 nM and about 20 nM, between about 20 nM and about 50 nM, between about 50 nM and about 100 nM, between about 100 nM and about 200 nM, between about 200 nM and about 500 nM, between about 500 nM and about 1000 nM, or more than about 1,000 nM.

In some embodiments, the ratio between the soluble binding agent molecules and the immobilized macromolecule, e.g., polypeptides, can be at any suitable range, e.g., at about 0.00001:1, about 0.0001:1, about 0.001:1, about 0.01:1, about 0.1:1, about 1:1, about 2:1, about 5:1, about 10:1, about 15:1, about 20:1, about 25:1, about 30:1, about 35:1, about 40:1, about 45:1, about 50:1, about 55:1, about 60:1, about 65:1, about 70:1, about 75:1, about 80:1, about 85:1, about 90:1, about 95:1, about 100:1, about 10₄:1, about 10₅:1, about 10₆:1, or higher, or any ratio in between the above listed ratios. Higher ratios between the soluble binding agent molecules and the immobilized polypeptide(s) and/or the nucleic acids can be used to drive the binding and/or the coding tag information transfer to completion. This may be particularly useful for detecting and/or analyzing low abundance polypeptides in a sample.

In some embodiments, the binding agent is compatible for use in temperatures used in the macromolecule analysis assay. The binding agent may exhibit characteristics desired such as stability, solubility, and compatibility with other components of the macromolecule analysis assay. In some examples, the binding agent is compatible with the surface which is joined (directly or indirectly) to the macromolecules (e.g., polypeptides). In some embodiments, the binding agents exhibit low non-specific binding to the surface.

2. Amino Acid Cleavage

In some embodiments, following the transfer of identifying information from a coding tag to a recording tag, at least one terminal amino acid is removed, cleaved, or eliminated from the peptide. In some embodiments, the at least one removed terminal amino acid comprises a modified amino acid. In some embodiments, the at least one removed terminal amino acid comprises an unmodified amino acid. In embodiments relating to methods of analyzing peptides or polypeptides using a degradation based approach, following contacting and binding of a first binding agent to an N-terminal amino acid (e.g., NTAA) of a peptide of n amino acids and transfer of the first binding agent's coding tag information to a nucleic acid associated with the peptide, thereby generating a first order extended nucleic acid (e.g., on the recording tag), the NTAA is eliminated or removed as described herein. Removal of the N-labeled NTAA by contacting with an enzyme and/or chemical reagent(s) converts the n-1 amino acid of the peptide to an N-terminal amino acid, which is referred to herein as an n-1 NTAA. A second binding agent is contacted with the peptide and binds to the n-1 NTAA, and the second binding agent's coding tag information is transferred to the first order extended nucleic acid thereby generating a second order extended nucleic acid (e.g., for generating a concatenated n^{th} order extended nucleic acid representing the peptide). Elimination of the n-1 labeled NTAA converts the n-2 amino acid of the peptide to an N-terminal amino acid, which is referred to herein as n-2 NTAA. Additional binding, transfer, labeling, and removal, can occur as described above up to n amino acids to generate an n-order extended nucleic acid or n separate extended nucleic acids, which collectively represent the peptide. As used herein, an n “order” when used in reference to a binding agent, coding tag, or extended nucleic acid, refers to the n binding cycle, wherein the binding agent and its associated coding tag is used or the n binding cycle where the extended nucleic acid is created (e.g. on recording tag). In some embodiments, steps including the NTAA in the described exemplary approach can be performed instead with a C terminal amino acid (CTAA).

In certain embodiments relating to analyzing peptides, following binding of a terminal amino acid (N-terminal or C-terminal) by a binding agent and transfer of coding tag information to a recording tag, the terminal amino acid is removed or cleaved from the peptide to expose a new terminal amino acid. In some embodiments, the terminal amino acid is an NTAA. In other embodiments, the terminal amino acid is a CTAA. Cleavage of a terminal amino acid can be accomplished by any number of known techniques, including chemical cleavage and enzymatic cleavage. In some embodiments, applying microwave energy to the sample (e.g., polypeptides) may accelerate the reaction for removing the terminal amino acid from the peptide. In some cases, applying microwave energy during one or more steps of the methods for macromolecule analysis may reduce overall cycle time of the assay.

In some embodiments, an engineered enzyme that catalyzes or reagent that promotes the removal of a labeled terminal amino acid is used. For example, the terminal amino acid is labeled with a PTC, a modified-PTC, a Cbz, a DNP, a SNP, an acetyl, a guanidinyl, amino guanidinyl, or a heterocyclic imine (e.g., heterocyclic methanimine). In some embodiments, the terminal amino acid is removed or eliminated using any of the methods as described in International Patent Publication No. WO 2019/089846.

Enzymatic cleavage of a terminal amino acid may be accomplished by an aminopeptidase or other peptidases (e.g., a carboxypeptidase, dipeptidyl peptidase, dipeptidyl aminopeptidase, or variant, mutant, or modified protein thereof). Aminopeptidases naturally occur as monomeric and multimeric enzymes, and may be metal or ATP-dependent. In some cases, natural aminopeptidases have very limited specificity, and generically cleave N-terminal amino acids in a processive manner, cleaving one amino acid off after another (Kishor et al., 2015, Anal. Biochem. 488:6-8). For the methods described here, aminopeptidases (e.g., metalloenzymatic aminopeptidase) may be engineered to possess specific binding or catalytic activity to the NTAA only when modified with an N-terminal label. For example, an aminopeptidase may be engineered such than it only cleaves an N-terminal amino acid if it is modified by a group such as PTC, modified-PTC, Cbz, DNP, SNP, acetyl, guanidinyl, amino guanidinyl, heterocyclic methanimine, etc. In this way, the aminopeptidase cleaves only a single amino acid at a time from the N-terminus, and allows control of the degradation cycle. In some embodiments, the modified aminopeptidase is non-selective as to amino acid residue identity while being selective for the N-terminal label. In other embodiments, the modified aminopeptidase is selective for both amino acid residue identity and the N-terminal label. Engineered aminopeptidase mutants that bind to and cleave individual or small groups of labelled (biotinylated) NTAAs have been described (see, International Patent Publication No. WO2010/065322). In some cases, residue specific aminopeptidases have been identified (Eriquez et al., J. Clin. Microbiol. 1980, 12:667-71; Wilce et al., 1998, Proc. Natl. Acad. Sci. USA 95:3472-3477; Liao et al., 2004, Prot. Sci. 13:1802-10). Control of the stepwise degradation of the N-terminus of the peptide may be achieved by using engineered aminopeptidases that are only active (e.g., binding activity or catalytic activity) in the presence of the label.

In certain embodiments, the aminopeptidase may be engineered to be non-specific, such that it does not selectively recognize one particular amino acid over another, but rather just recognizes the labeled N-terminus. In yet another embodiment, cyclic cleavage is attained by using an engineered acylpeptide hydrolase (APH) to cleave an acetylated NTAA. In yet another embodiment, amidination (guanidinylation) of the NTAA is employed to enable mild cleavage of the labeled NTAA using NaOH (Hamada, (2016) Bioorg Med Chem Lett 26(7): 1690-1695).

For embodiments relating to CTAA binding agents, methods of cleaving CTAA from peptides are also known in the art. For example, U.S. Pat. No. 6,046,053 discloses a method of reacting the peptide or protein with an alkyl acid anhydride to convert the carboxy-terminal into oxazolone, liberating the C-terminal amino acid by reaction with acid and alcohol or with ester. Enzymatic cleavage of a CTAA may also be accomplished by a carboxypeptidase. Several carboxypeptidases exhibit amino acid preferences, e.g., carboxypeptidase B preferentially cleaves at basic amino acids, such as arginine and lysine. As described above, carboxypeptidases may also be modified in the same fashion as aminopeptidases to engineer carboxypeptidases that specifically bind to CTAAs having a C-terminal label. In this way, the carboxypeptidase cleaves only a single amino acid at a time from the C-terminus, and allows control of the degradation cycle. In some embodiments, the modified carboxypeptidase is non-selective as to amino acid residue identity while being selective for the C-terminal label. In other embodiments, the modified carboxypeptidase is selective for both amino acid residue identity and the C-terminal label.

In some embodiments, the removed amino acid is a modified amino acid. For example, the reagent may comprise an enzymatic or chemical reagent to remove one or more terminal amino acid. For example, in some cases, the reagent for eliminating the functionalized NTAA is a carboxypeptidase, or aminopeptidase, or dipeptidyl peptidase, dipeptidyl aminopeptidase, or variant, mutant, or modified protein thereof; a hydrolase or variant, mutant, or modified protein thereof, mild Edman degradation; Edmanase enzyme; TFA, a base; or any combination thereof. In some cases, the removing reagent comprises trifluoroacetic acid or hydrochloric acid. In some examples, the removing reagent comprises acylpeptide hydrolase (APH). In some embodiments, the removing reagent includes a carboxypeptidase or an aminopeptidase or a variant, mutant, or modified protein thereof, a hydrolase or a variant, mutant, or modified protein thereof, a mild Edman degradation reagent; an Edmanase enzyme; anhydrous TFA, a base; or any combination thereof. In some embodiments, the mild Edman degradation uses a dichloro or monochloro acid; the mild Edman degradation uses TFA, TCA, or DCA; or the mild Edman degradation uses triethylamine, triethanolamine, or triethylammonium acetate (Et₃NHOAc).

The chemical reagent used for removing one or more amino acids may be compatible with the materials used in the assay, for example, with the nucleic acid recording tags. In some cases, the chemical reagent or treatment used is mild and the conditions are stable for the nucleic acid recording tags over one or more cycles of treatment.

In some cases, the reagent for removing the amino acid comprises a base. In some embodiments, the base is a hydroxide, an alkylated amine, a cyclic amine, a carbonate buffer, trisodium phosphate buffer, or a metal salt. In some examples, the hydroxide is sodium hydroxide; the alkylated amine is selected from methylamine, ethylamine, propylamine, dimethylamine, diethylamine, dipropylamine, trimethylamine, triethylamine, tripropylamine, cyclohexylamine, benzylamine, aniline, diphenylamine, N,N-diisopropylethylamine (DIPEA), and lithium diisopropylamide (LDA); the cyclic amine is selected from pyridine, pyrimidine, imidazole, pyrrole, indole, piperidine, pyrrolidine, 1,8-diazabicyclo[5.4.0]undec-7-ene (DBU), and 1,5-diazabicyclo[4.3.0]non-5-ene (DBN); the carbonate buffer comprises sodium carbonate, potassium carbonate, calcium carbonate, sodium bicarbonate, potassium bicarbonate, or calcium bicarbonate; the metal salt comprises silver; or the metal salt is AgClO₄.

In some embodiments, the method further includes contacting the polypeptide with a peptide coupling reagent. In some embodiments, the peptide coupling reagent is a carbodiimide compound. In some examples, the carbodiimide compound is diisopropylcarbodiimide (DIC) or 1-ethyl-3-(3-dimethylaminopropyl)carbodiimide (EDC).

### III. Processing and Analysis

The apparatus described in Section I and automated methods in Section II can be used to perform one or more steps of a macromolecules analysis assay that generates an extended recoding tag. In some embodiments, the extended recording tag generated comprises identifying information from one or more coding tags. In some embodiments, the extended recording tag(s) (or a portion thereof) are amplified and/or copied prior to determining at least a portion of the sequence of the extended recording tag(s). In some embodiments, the extended recording tag(s) (or a portion thereof) are released from the macromolecule (e.g., polypeptide) prior to analysis of the extended recording tag(s). In some embodiments, the method includes collecting extended recording tags. In some embodiments, the amplification, release, processing, and/or collection of extended recording tags may be performed in an automated manner, (e.g., by using the described apparatus). In some cases, the sample is treating with a cleaving reagent prior to collection. For example, the extended recording tags or a portion thereof, are cleaved from the macromolecule prior to collection. In some embodiments, the analysis of the extended recording tag is performed after the steps performed using the apparatus of Section I or the methods in Section II. In some cases, the analysis is not performed using the apparatus described in Section I. For example, the sample or a portion thereof containing the extended recording tags is removed from the apparatus prior to analysis steps.

The length of the final extended nucleic acids (e.g., on the extended recording tag) generated by the methods described herein is dependent upon multiple factors, including the length of the coding tag (e.g., encoder sequence and spacer) and the length of any other of the nucleic acids (e.g., on the recording tag, optionally including any unique molecular identifier, spacer, universal priming site, barcode(s), or combinations thereof), the number of transfer cycles performed, and whether coding tags from each binding cycle are transferred to the same extended nucleic acid or to multiple extended nucleic acids.

In some embodiments, a recording tag comprises from 5′ to 3′ direction: a universal forward (or 5′) priming sequence, a UMI, and a spacer sequence. In some embodiments, a recording tag comprises from 5′ to 3′ direction: a universal forward (or 5′) priming sequence, an optional UMI, a barcode (e.g., sample barcode, partition barcode, compartment barcode, spatial barcode, or any combination thereof), and a spacer sequence. In some other embodiments, a recording tag comprises from 5′ to 3′ direction: a universal forward (or 5′) priming sequence, a barcode (e.g., sample barcode, partition barcode, compartment barcode, spatial barcode, or any combination thereof), an optional UMI, and a spacer sequence.

After the transfer of the final tag information to the extended recording tag from a coding tag, the tag can be capped (e.g., end-capping as described in Example I) by addition of a universal reverse priming site via ligation, primer extension or other methods known in the art. In some embodiments, the universal forward priming site in the nucleic acid (e.g., on the recording tag) is compatible with the universal reverse priming site that is appended to the final extended nucleic acid. In some embodiments, a universal reverse priming site is an Illumina P7 primer (5′-CAAGCAGAAGACGGCATACGAGAT-3′—SEQ ID NO:2) or an Illumina P5 primer (5′-AATGATACGGCGACCACCGA-3′—SEQ ID NO:1). The sense or antisense P7 may be appended, depending on strand sense of the nucleic acid to which the identifying information from the coding tag is transferred to. In some embodiments, the capping sequence or sequences can be included with a coding tag(s). For example, the capping step can be performed as part of a final encoding step. An extended nucleic acid library can be cleaved or amplified directly from the solid support (e.g., beads) and used in traditional next generation sequencing assays and protocols. In some embodiments, the capping reaction is performed as a last step on the apparatus in an automated manner prior to releasing or collecting the extended recording tags.

In some embodiments, a primer extension reaction is performed on a library of single stranded extended nucleic acids (e.g., extended on the recording tag) to copy complementary strands thereof. The primer extension may be performed prior to or after the sample is removed from the sample container on the apparatus. In some embodiments, the peptide sequencing assay (e.g., ProteoCode assay), comprises several chemical and enzymatic steps in a cyclical progression. In some cases, one advantage of a single molecule assay is the robustness to reduce or minimize inefficiencies in the various cyclical chemical/enzymatic steps. In some embodiments, the use of cycle-specific barcodes present in the coding tag sequence may be advantageous.

Extended nucleic acids (e.g., extended recording tags) can be processed and analyzed using a variety of nucleic acid sequencing methods. In some embodiments, extended recording tags containing the information from one or more coding tags and any other nucleic acid components are processed and analyzed. In some embodiments, the collection of extended recording can be concatenated. In some embodiments, the extended recording tag can be amplified prior to determining the sequence. The processing of the extended recording tags may be performed prior to or after the sample is removed from the sample container.

A library of nucleic acids (e.g., extended nucleic acids) may be amplified in a variety of ways. A library of nucleic acids (e.g., recording tags comprising information from one or more probe tags) undergo exponential amplification, e.g., via PCR or emulsion PCR. Emulsion PCR is known to produce more uniform amplification (Hori, Fukano et al., Biochem Biophys Res Commun (2007) 352(2): 323-328). Alternatively, a library of nucleic acids (e.g., extended nucleic acids) may undergo linear amplification, e.g., via in vitro transcription of template DNA using T7 RNA polymerase. The library of nucleic acids (e.g., extended nucleic acids) can be amplified using primers compatible with the universal forward priming site and universal reverse priming site contained therein. A library of nucleic acids (e.g., the recording tag) can also be amplified using tailed primers to add sequence to either the 5′-end, 3′-end or both ends of the extended nucleic acids. Sequences that can be added to the termini of the extended nucleic acids include library specific index sequences to allow multiplexing of multiple libraries in a single sequencing run, adaptor sequences, read primer sequences, or any other sequences for making the library of extended nucleic acids compatible for a sequencing platform. An example of a library amplification in preparation for next generation sequencing is as follows: a 20 μl PCR reaction volume is set up using an extended nucleic acid library eluted from ˜1 mg of beads (˜10 ng), 200 μM dNTP, 1 μM of each forward and reverse amplification primers, 0.5 μl (1 U) of Phusion Hot Start enzyme (New England Biolabs) and subjected to the following cycling conditions: 98° C. for 30 sec followed by 20 cycles of 98° C. for 10 sec, 60° C. for 30 sec, 72° C. for 30 sec, followed by 72° C. for 7 min, then hold at 4° C.

In certain embodiments, either before, during or following amplification, the library of nucleic acids (e.g., extended nucleic acids) can undergo target enrichment. In some embodiments, target enrichment can be used to selectively capture or amplify extended nucleic acids representing macromolecules (e.g., polypeptides) of interest from a library of extended nucleic acids before sequencing. In some aspects, target enrichment for protein sequencing is challenging because of the high cost and difficulty in producing highly-specific binding agents for target proteins. In some cases, antibodies are notoriously non-specific and difficult to scale production across thousands of proteins. In some embodiments, the methods of the present disclosure circumvent this problem by converting the protein code into a nucleic acid code which can then make use of a wide range of targeted DNA enrichment strategies available for DNA libraries. In some cases, peptides of interest can be enriched in a sample by enriching their corresponding extended nucleic acids. Methods of targeted enrichment are known in the art, and include hybrid capture assays, PCR-based assays such as TruSeq custom Amplicon (Illumina), padlock probes (also referred to as molecular inversion probes), and the like (see, Mamanova et al., (2010) Nature Methods 7: 111-118; Bodi et al., J. Biomol. Tech. (2013) 24:73-86; Ballester et al., (2016) Expert Review of Molecular Diagnostics 357-372; Mertes et al., (2011) Brief Funct. Genomics 10:374-386; Nilsson et al., (1994) Science 265:2085-8; each of which are incorporated herein by reference in their entirety).

In one embodiment, a library of nucleic acids (e.g., extended nucleic acids) is enriched via a hybrid capture-based assay. In a hybrid-capture based assay, the library of extended nucleic acids is hybridized to target-specific oligonucleotides that are labeled with an affinity tag (e.g., biotin). Extended nucleic acids hybridized to the target-specific oligonucleotides are “pulled down” via their affinity tags using an affinity ligand (e.g., streptavidin coated beads), and background (non-specific) extended nucleic acids are washed away. The enriched extended nucleic acids (e.g., extended nucleic acids) are then obtained for positive enrichment (e.g., eluted from the beads). In some embodiments, oligonucleotides complementary to the corresponding extended nucleic acid library representations of peptides of interest can be used in a hybrid capture assay. In some embodiments, sequential rounds or enrichment can also be carried out, with the same or different bait sets.

To enrich the entire length of a polypeptide in a library of extended nucleic acids representing fragments thereof (e.g., peptides), “tiled” bait oligonucleotides can be designed across the entire nucleic acid representation of the protein.

In another embodiment, primer extension and ligation-based mediated amplification enrichment (AmpliSeq, PCR, TruSeq TSCA, etc.) can be used to select and module fraction enriched of library elements representing a subset of polypeptides. Competing oligonucleotides can also be employed to tune the degree of primer extension, ligation, or amplification. In the simplest implementation, this can be accomplished by having a mix of target specific primers comprising a universal primer tail and competing primers lacking a 5′ universal primer tail. After an initial primer extension, only primers with the 5′ universal primer sequence can be amplified. The ratio of primer with and without the universal primer sequence controls the fraction of target amplified. In other embodiments, the inclusion of hybridizing but non-extending primers can be used to modulate the fraction of library elements undergoing primer extension, ligation, or amplification.

Targeted enrichment methods can also be used in a negative selection mode to selectively remove extended nucleic acids from a library before sequencing. Examples of undesirable extended nucleic acids that can be removed are those representing over abundant polypeptide species, e.g., for proteins, albumin, immunoglobulins, etc.

A competitor oligonucleotide bait, hybridizing to the target but lacking a biotin moiety, can also be used in the hybrid capture step to modulate the fraction of any particular locus enriched. The competitor oligonucleotide bait competes for hybridization to the target with the standard biotinylated bait effectively modulating the fraction of target pulled down during enrichment. The ten orders dynamic range of protein expression can be compressed by several orders using this competitive suppression approach, especially for the overly abundant species such as albumin. Thus, the fraction of library elements captured for a given locus relative to standard hybrid capture can be modulated from 100% down to 0% enrichment.

Additionally, library normalization techniques can be used to remove overly abundant species from the extended nucleic acid library. This approach works best for defined length libraries originating from peptides generated by site-specific protease digestion such as trypsin, LysC, GluC, etc. In one example, normalization can be accomplished by denaturing a double-stranded library and allowing the library elements to re-anneal. The abundant library elements re-anneal more quickly than less abundant elements due to the second-order rate constant of bimolecular hybridization kinetics (Bochman, Paeschke et al. 2012). The ssDNA library elements can be separated from the abundant dsDNA library elements using methods known in the art, such as chromatography on hydroxyapatite columns (VanderNoot, et al., 2012, Biotechniques 53:373-380) or treatment of the library with a duplex-specific nuclease (DSN) from Kamchatka crab (Shagin et al., (2002) Genome Res. 12:1935-42) which destroys the dsDNA library elements.

Any combination of fractionation, enrichment, and subtraction methods, of the polypeptides before attachment to the solid support and/or of the resulting extended nucleic acid library can economize sequencing reads and improve measurement of low abundance species. In some embodiments, a library of nucleic acids (e.g., extended nucleic acids) is concatenated by ligation or end-complementary PCR to create a long DNA molecule comprising multiple different extended recorder tags (Du et al., (2003) BioTechniques 35:66-72; Muecke et al., (2008) Structure 16:837-841; U.S. Pat. No. 5,834,252, each of which is incorporated by reference in its entirety). This embodiment is preferable for nanopore sequencing in which long strands of DNA are analyzed by the nanopore sequencing device.

In some embodiments, the recording tag or extended recording tag comprising information from one or more coding tags is analyzed and/or sequenced. In some cases, analysis and/or sequencing of the recording tags or extended recording tags is performed using a separate instrument. In some cases, analysis and/or sequencing is performed after the removal of the sample or a portion thereof containing the of the recording tags or extended recording tags from the apparatus. In some embodiments, direct single molecule analysis is performed on the nucleic acids (e.g., extended nucleic acids) (see, e.g., Harris et al., (2008) Science 320:106-109). The nucleic acids (e.g., extended nucleic acids) can be analyzed directly on the solid support, such as a flow cell or beads that are compatible for loading onto a flow cell surface (optionally microcell patterned), wherein the flow cell or beads can integrate with a single molecule sequencer or a single molecule decoding instrument. For single molecule decoding, hybridization of several rounds of pooled fluorescently-labeled of decoding oligonucleotides (Gunderson et al., (2004) Genome Res. 14:970-7) can be used to ascertain both the identity and order of the coding tags within the extended nucleic acids (e.g., on the recording tag). In some embodiments, the binding agents may be labeled with cycle-specific coding tags as described above (see also, Gunderson et al., (2004) Genome Res. 14:970-7).

In some examples, the labels can be read out using traditional arrays or sequence-based methods. The methods described herein can be used in conjunction with a variety of sequencing techniques. In some embodiments, the process to determine the nucleotide sequence of a target nucleic acid can be an automated process. Examples of sequencing methods include, but are not limited to, chain termination sequencing (Sanger sequencing); next generation sequencing methods, such as sequencing by synthesis, sequencing by ligation, sequencing by hybridization, polony sequencing, ion semiconductor sequencing, and pyrosequencing; and third generation sequencing methods, such as single molecule real time sequencing, nanopore-based sequencing, duplex interrupted sequencing, and direct imaging of DNA using advanced microscopy. In some embodiments, suitable sequencing methods for use in the invention include, but are not limited to, sequencing by hybridization, sequencing by synthesis technology (e.g., HiSeq™ and Solexa™, Illumina), SMRT™ (Single Molecule Real Time) technology (Pacific Biosciences), true single molecule sequencing (e.g., HeliScope™, Helicos Biosciences), massively parallel next generation sequencing (e.g., SOLiD™, Applied Biosciences; Solexa and HiSeq™, Illumina), massively parallel semiconductor sequencing (e.g., Ion Torrent), pyrosequencing technology (e.g., GS FLX and GS Junior Systems, Roche/454), nanopore sequence (e.g., Oxford Nanopore Technologies).

Examples of next generation sequencing methods include sequencing by synthesis, sequencing by ligation, sequencing by hybridization, polony sequencing, ion semiconductor sequencing, and pyrosequencing. By attaching primers to a solid substrate and a complementary sequence to a nucleic acid molecule, a nucleic acid molecule can be hybridized to the solid substrate via the primer and then multiple copies can be generated in a discrete area on the solid substrate by using polymerase to amplify (these groupings are sometimes referred to as polymerase colonies or polonies). Consequently, during the sequencing process, a nucleotide at a particular position can be sequenced multiple times (e.g., hundreds or thousands of times)—this depth of coverage is referred to as “deep sequencing.” Examples of high throughput nucleic acid sequencing technology include platforms provided by Illumina, BGI, Qiagen, Thermo-Fisher, and Roche, including formats such as parallel bead arrays, sequencing by synthesis, sequencing by ligation, capillary electrophoresis, electronic microchips, “biochips,” microarrays, parallel microchips, and single-molecule arrays, as reviewed by Service (Science (2006) 311:1544-1546).

Some embodiments of the sequencing methods described herein include sequencing by synthesis (SBS) technologies, for example, pyrosequencing techniques. Pyrosequencing detects the release of inorganic pyrophosphate (PPi) as particular nucleotides are incorporated into the nascent strand (Ronaghi et al, Analytical Biochemistry 242(1): 84-9 (1996); Ronaghi, M. Genome Res. 11(1):3-11 (2001); Ronaghi et al, Science 281(5375):363 (1998); U.S. Pat. Nos. 6,210,891; 6,258,568 and 6,274,320, each of which is incorporated by reference in its entirety).

In another exemplary type of SBS, cycle sequencing is accomplished by stepwise addition of reversible terminator nucleotides containing, for example, a cleavable or photobleachable dye label as described, for example, in U.S. Pat. Nos. 7,427,67, 7,414,1163 and 7,057,026, each of which is incorporated by reference in its entirety. This approach, which is being commercialized by Illumina Inc., is also described in International Patent Application Publication Nos. WO 91/06678 and WO 07/123744, each of which is incorporated by reference in its entirety. The availability of fluorescently-labeled terminators, in which both the termination can be reversed and the fluorescent label cleaved, facilitates efficient cyclic reversible termination (CRT) sequencing. Polymerases can also be co-engineered to efficiently incorporate and extend from these modified nucleotides.

Additional exemplary SBS systems and methods which can be utilized with the methods and compositions described herein are described in U.S. Patent Application Publication No. 2007/0166705, U.S. Patent Application Publication No. 2006/0188901, U.S. Pat. No. 7,057,026, U.S. Patent Application Publication No. 2006/0240439, U.S. Patent Application Publication No. 2006/0281109, International Patent Publication No. WO 05/065814, U.S. Patent Application Publication No. 2005/0100900, International Patent Publication No. WO 06/064199 and International Patent Publication No. WO 07/010251, each of which is incorporated by reference in its entirety.

Some embodiments of the sequencing technology described herein can utilize sequencing by ligation techniques. Such techniques utilize DNA ligase to incorporate nucleotides and identify the incorporation of such nucleotides. Exemplary SBS systems and methods which can be utilized with the compositions and methods described herein are described in U.S. Pat. Nos. 6,969,488, 6,172,218, and 6,306,597, each of which is incorporated by reference in its entirety.

The sequencing methods described herein can be advantageously carried out in multiplex formats such that multiple different target nucleic acids are manipulated simultaneously. In particular embodiments, different target nucleic acids can be treated in a common reaction vessel or on a surface of a particular substrate. This allows convenient delivery of sequencing reagents, removal of unreacted reagents and detection of incorporation events in a multiplex manner. In embodiments using surface-bound target nucleic acids, the target nucleic acids can be in an array format. In an array format, the target nucleic acids can be typically coupled to a surface in a spatially distinguishable manner. For example, the target nucleic acids can be bound by direct covalent attachment, attachment to a bead or other particle or associated with a polymerase or other molecule that is attached to the surface. The array can include a single copy of a target nucleic acid at each site (also referred to as a feature) or multiple copies having the same sequence can be present at each site or feature. Multiple copies can be produced by amplification methods such as, bridge amplification or emulsion PCR as described in further detail herein.

In some embodiments, the analysis of the sequence information of any of the labels (e.g., in the extended recording tag), or any portion thereof (e.g., a universal primer, a spacer, a UMI, a barcode), can be done using a single-molecule sequencing method, such as a nanopore based sequencing technology. In one aspect, the single-molecule sequencing method is a direct single-molecule sequencing method. See International Patent Application Publication No WO 2017/125565 for certain aspects of exemplary nanopore based sequencing, the content of which is incorporated by reference in its entirety. Nanopore sequencing of DNA and RNA may be achieved by strand sequencing and/or exosequencing of DNA and RNA. Strand sequencing comprises methods whereby nucleotide bases of a sample polynucleotide strand are determined directly as the nucleotides of the polynucleotide template are threaded through a nanopore. Alternatively, strand sequencing of the polynucleotide strand determines the sequence of the template indirectly by determining nucleotides that are incorporated into a growing strand that is complementary to that of the sample template strand.

In some embodiments, DNA, e.g., single stranded DNA, may be sequenced by detecting tags of tagged nucleotides that are released from the nucleotide base as the nucleotide is incorporated by a polymerase into a strand complementary to that of a template associated with the polymerase in an enzyme-polymer complex. The single molecule nanopore-based sequencing by synthesis (Nano-SBS) technique that uses tagged nucleotides is described, for example, in International Patent Application Publication No WO2014/074727, which is incorporated by reference in its entirety. Accordingly, in some embodiments, the enzyme-polynucleotide complex that may be attached to the inserted nanopore may be a DNA polymerase-DNA complex. In some embodiments, the DNA polymerase-DNA complex may be attached to a wild-type or variant monomeric nanopore. In some embodiments, the DNA polymerase-DNA complex may be attached to a wild-type, variant, or modified variant homo-oligomeric nanopore. In some embodiments, the DNA polymerase-DNA complex may be attached to a wild-type, a variant, or a modified variant hetero-oligomeric nanopore. In some embodiments, the DNA polymerase-DNA complex may be attached to a wild-type, variant, or modified variant aHL nanopore. In other embodiments, the DNA polymerase-DNA complex may be attached to a wild-type OmpG nanopore or variants thereof.

In other embodiments, the enzyme-polynucleotide complex may be an RNA polymerase-RNA complex. The RNA polymerase-RNA complex may be attached to a wild-type or variant oligomeric or monomeric nanopore. In some embodiments, the RNA polymerase-RNA complex is attached to a wild-type or variant OmpG nanopore. In other embodiments, the RNA polymerase-RNA complex is attached to a wild-type or variant aHL nanopore. In yet other embodiments, the enzyme-polynucleotide complex may be a reverse transcriptase-RNA complex. The reverse transcriptase-RNA complex may be attached to a wild-type or variant oligomeric or monomeric nanopore. In some embodiments, the reverse transcriptase-RNA complex is attached to a wild-type or variant OmpG nanopore. In other embodiments, the reverse transcriptase-RNA complex is attached to a wild-type or variant aHL nanopore. In some embodiments, individual nucleic acids may be sequenced by the identification of nucleoside 5′-monophosphates as they are released by processive exonucleases (Astier et al., 2006, J Am Chem Soc 128:1705-1710). Accordingly, in some embodiments, the enzyme-polynucleotide complex that may be attached to the inserted nanopore may be an exonuclease-polynucleotide complex. In some embodiments, the exonuclease-polynucleotide complex may be attached to a wild-type or variant monomeric nanopore. In some embodiments, the exonuclease-polynucleotide complex may be attached to a wild-type or variant homo-oligomeric nanopore. In some embodiments, the exonuclease-polynucleotide complex may be attached to a wild-type or variant hetero-oligomeric nanopore. In some embodiments, the exonuclease-polynucleotide complex may be attached to a wild-type aHL nanopore or variants thereof. In other embodiments, the exonuclease-polynucleotide complex may be attached to a wild-type OmpG nanopore or variants thereof.

In some embodiments, a non-nucleic acid polymer may also be move through a nanopore and be sequenced. For example, proteins and polypeptides can move through nanopores, and sequencing of a protein or a polynucleotide using a nanopore can be performed by controlling the unfolding and translocation of the protein through the nanopore. The controlled unfolding and subsequent translocation can be achieved by the action of an unfoldase enzyme coupled to the protein to be sequenced (see e.g., Nivala et al., 2013, Nature Biotechnol 31:247-250). In some embodiments, the enzyme-polymer complex that is attached to the nanopore in the membrane may be an enzyme-polypeptide complex, e.g., an unfoldase-protein complex. In some embodiments, the unfoldase-protein complex may be attached to a wild-type or variant monomeric nanopore. In some embodiments, the unfoldase-protein complex may be attached to a wild-type or variant homo-oligomeirc nanopore. In some embodiments, the unfoldase-protein complex may be attached to a wild-type or variant hetero-oligomeric nanopore. In some embodiments, the unfoldase-protein complex may be attached to a wild-type aHL nanopore or variants thereof. In other embodiments, the unfoldase-protein complex may be attached to a wild-type OmpG nanopore or variants thereof.

In some embodiments, other non-nucleic acid polymers may also be sequenced, for example, by moving through a nanopore. For example, WO 1996013606 A1 describes exo-sequencing of saccharide material, such as a polysaccharide including heparan sulphate (HS) and heparin, and U.S. Pat. No. 8,846,363 B2 discloses enzymes (such as a sulfatase from Flavobacterium heparinum) that can be applied (e.g., in tandem) toward the exo-sequencing of a polysaccharide, such as heparin-derived oligosaccharides. Both patent documents are incorporated herein by reference in their entireties for all purposes.

In some embodiments, the information from analysis (e.g., sequencing) of at least a portion of the extended recording tag can be used to associate the sequences determined to corresponding a polypeptide and align to the proteome. In some cases, following sequencing of the nucleic acid libraries (e.g., of extended nucleic acids), the resulting sequences can be collapsed by their UMIs and then associated to their corresponding polypeptides and aligned to the totality of the proteome. In some cases, resulting sequences can also be collapsed by their compartment tags and associated to their corresponding compartmental proteome, which in a particular embodiment contains only a single or a very limited number of protein molecules. In some embodiments, both protein identification and quantification can be derived from this digital peptide information.

The methods disclosed herein can be used for preparing and treating macromolecules for analysis, including detection, quantitation and/or sequencing, of a plurality of macromolecules simultaneously (multiplexing). Multiplexing as used herein refers to analysis of a plurality of macromolecules (e.g. polypeptides) in the same assay. The plurality of macromolecules can be derived from the same sample or different samples. The plurality of macromolecules can be derived from the same subject or different subjects. The plurality of macromolecules that are analyzed can be different macromolecules, or the same macromolecule derived from different samples. A plurality of macromolecules includes 2 or more macromolecules, 5 or more macromolecules, 10 or more macromolecules, 50 or more macromolecules, 100 or more macromolecules, 500 or more macromolecules, 1000 or more macromolecules, 5,000 or more macromolecules, 10,000 or more macromolecules, 50,000 or more macromolecules, 100,000 or more macromolecules, 500,000 or more macromolecules, or 1,000,000 or more macromolecules.

### IV. Exemplary Uses and Applications

Provided herein are exemplary methods for treating and preparing macromolecules for an analysis assay. In some embodiments, one or more steps of the provided methods may be performed in an automated manner and are useful for preforming high-throughput sample processing. In some embodiments, the apparatus and/or automated methods are configured to integrate an aqueous-phase biochemical reaction and an organic chemical reaction into a cyclic process, e.g., a cyclic process for converting a polypeptide or a peptide sequence into a DNA library for NGS analysis. The apparatus and methods described herein can generate an output sample (e.g., an output sample comprising a DNA library or an encoded library) that is compatible for analysis with a DNA sequencer, e.g., a general purpose DNA sequencer (NGS). The use of the apparatus for the treatment and preparation of the macromolecules described herein enables downstream analysis of single molecules, e.g., sequence of individual peptides, polypeptides, or proteins.

In some embodiments, the use of the apparatus provided herein allows greater temperature control. In some aspects, the integrated system provides an enclosed environment for performing the steps of the macromolecule analysis assay. The integrated system may provide certain advantages. For example, the temperature control can be more precise, temperature changes can be more accurate and efficient, and temperature can be more uniformly controlled (e.g. between samples).

In some embodiments, the apparatus and/or automated methods used for treating and preparing macromolecules for an analysis assay may be operated without real-time control or without precise real-time control. For example, using the apparatus and/or automated methods, different processes can be performed in a single operation without user intervention throughout the process, as compared to a manual method for performing the macromolecule analysis assay. In some embodiments, automation may be achieved by using a control program run by a control unit of the apparatus to carry out desired reactions in sequence. For example, the control program delivers and removes reagents to and from the sample container in a cyclic manner. In some cases, the program sets the temperature of the reactions/incubations of the sample with various reagents for a predetermined or desired amount of time. Various loops of the program in whole or in part can be carried out, for the repeated steps of the methods. The use of the control program and apparatus allows the sample to be prepared and treated with minimal input and physical action required from the user. For example, a user may be able to load the apparatus with the appropriate reagents and samples and allow the rest of the processes to be carried out automatically.

In some embodiments, performing the step of a) providing a non-planar sample container comprising a sample comprising a macromolecule, e.g., a polypeptide, and an associated recording tag joined to a solid support to the apparatus is automated and/or controlled by the control unit. In some embodiments, performing the step of b) providing a reagent to separate reagent reservoirs of said apparatus is automated and/or controlled by the control unit, e.g., providing a binding agent, reagents for transferring information, optionally providing reagents for removing a terminal amino acid of a polypeptide, reagents for a capping reaction, and/or a reagent for modifying a terminal amino acid of a polypeptide. In some embodiments, performing the step of c) delivering the binding agent from the reagent reservoir to the sample container, wherein the binding agent comprises a coding tag with identifying information regarding the binding agent is automated and/or controlled by the control unit. In some embodiments, performing the step of d) delivering the reagents for transferring information from the reagent reservoir to the sample container to transfer information from the coding tag of the binding agent to the recording tag to generate an extended recording tag is automated and/or controlled by the control unit. In some embodiments, performing the step of e) delivering the reagents for removing a terminal amino acid of a polypeptide from the reagent reservoir to the sample container to remove the terminal amino acid is automated and/or controlled by the control unit. In some embodiments, performing the step of f) delivering the reagents for a capping reaction from the reagent reservoir to the sample container is automated and/or controlled by the control unit. In some embodiments, delivering the reagent for modifying a terminal amino acid of a polypeptide to the sample container is automated and/or controlled by the control unit. In some cases, at least one of the steps c)-f) is conducted with one or more controlled flow rates. In some embodiments, two or more of the steps c)-f) are controlled by the control unit. In some examples, two, three, four, five or all of the steps a)-f) are automated. In some embodiments, any of steps c) to f) comprises incubating the sample with the provided reagent. In some examples, any of steps c) to f) comprises incubating the sample with the provided reagent and adjusting the temperature of the sample container during the incubation.

In some embodiments, the use of the apparatus and automated methods provided herein allows advantages for delivery of reagents and wash buffers. In some cases, it may be desirable to perform more stringent washing (e.g. to remove binding agents or other reagents), thus increasing the specificity of the assay. In some cases, the use of the apparatus and automated methods provided herein allows more reproducible sample treatment, finer control of volume delivery, and control of flow rates. The control program also allows the ability to program more complex washes or reagent delivery to the sample container. For example, various flow rates may be applied in sequence as controlled by the control program.

In some embodiments, compared to a manual method for treating and preparing the sample for a macromolecule analysis assay, a greater number of samples can be processed in parallel using the provided apparatus and methods. In some embodiments, the provided apparatus and methods enable high-throughput sample processing with greater control, reproducibility, and robustness. In some aspects, the macromolecule analysis assay is less restrictive when performed in an automated manner. For example, processes may be extended or repeated if the time required is not a limiting factor for the assay. In some cases, sample to sample variation can be also decreased when the assay is performed in an automated manner or using the provided apparatus. In some cases, the user may also barcode samples and combine the samples to achieve even greater throughput.

### V. Exemplary Embodiments

Among the provided embodiments are:

1. An apparatus for automated treatment of a sample containing an immobilized macromolecule, which apparatus comprises:
one or more non-planar sample container(s) with a volume equal to or less than about 20 mL, wherein at least one of said sample container(s) is subjected to temperature control and configured for allowing fluid flow-through, or a holder or space configured for holding said sample container(s);
a plurality of reagent reservoirs for containing a respective reagent, wherein at least one of said reagent reservoirs is subjected to temperature control, or a holder or space configured for holding said reagent reservoir(s);
a plurality of valves connected in a supply line having an upstream end and a downstream end, wherein at least one or each of said valves is positionable to provide alternate flow paths therethrough; and
a control unit to control delivery of said one or more reagent(s) to said sample container(s),
wherein:
delivery of said one or more reagent is individually addressable,
said supply line connects said reagent reservoirs to said sample container(s) and said reagent reservoirs are fluidically connected to said sample container(s), and
at least temperature control of said sample container(s), temperature control of said reagent reservoir(s), positioning of said valve(s) and/or delivery of said one or more reagent(s) to said sample container(s) is automated and controlled by said control unit.

2. The apparatus of embodiment 1, wherein at least one of the sample container(s) and/or at least one of the reagent reservoirs is subjected to active heating and/or active cooling.

3. The apparatus of embodiment 1 or 2, wherein the temperature of the sample container(s) subjected to temperature control and the temperature of the reagent reservoir(s) subjected to temperature control are individually controlled by the control unit.

4. The apparatus of embodiment 3, wherein the sample container(s) subjected to temperature control and the reagent reservoir(s) subjected to temperature control are housed in separate thermal blocks.

5. The apparatus of any one of embodiments 1-4, which further comprises a means for moving the one or more reagent, e.g., the one or more reagent liquid.

6. The apparatus of embodiment 5, wherein the means for moving one or more reagent or reagent liquid comprises a single pump.

7. The apparatus of embodiment 5, wherein the means for moving one or more reagent or reagent liquid comprises a plurality of pumps.

8. The apparatus of embodiment 6 or 7, wherein the pump(s) is integrated into the apparatus.

9. The apparatus of any one of embodiments 1-8, which further comprises a waste outlet and/or a waste container.

10. The apparatus of embodiment 9, wherein the apparatus comprises more than one waste container.

11. The apparatus of any one of embodiments 1-10, wherein the apparatus is configured to hold one or more of:
a reagent reservoir with a volume ranging from about 5 μL to about 50 μL;
a reagent reservoir with a volume ranging from about 50 μL to about 200 μL;
a reagent reservoir with a volume ranging from about 200 μL to about 1 mL;
a reagent reservoir with a volume ranging from about 1 mL to about 50 mL;
a reagent reservoir with a volume ranging from about 50 mL to about 500 mL;
a reagent reservoir with a volume ranging from about 500 mL to about 1 L; and/or
a reagent reservoir with a volume ranging from about 1 L to about 100 L.

12. The apparatus of any one of embodiments 1-11, wherein the apparatus is configured to hold at least 5 reagent reservoirs.

13. The apparatus of any one of embodiments 1-11, wherein the apparatus is configured to hold at least 10 reagent reservoirs.

14. The apparatus of any one of embodiments 1-11, wherein the apparatus is configured to hold at least 20 reagent reservoirs.

15. The apparatus of any one of embodiments 1-14, wherein the volume of at least one of the sample container(s) is equal to or less than about 10 mL

16. The apparatus of any one of embodiments 1-15, wherein the apparatus is configured to hold a single sample container, or to hold two or more sample containers.

17. The apparatus of any one of embodiments 1-16, wherein the sample container(s) has an inlet for the delivery of reagents and an outlet for evacuation of reagents.

18. The apparatus of embodiment 17, wherein the outlet of the sample container(s) is configured for draining liquid from the sample container(s) to a waste container.

19. The apparatus of any one of embodiments 10-18, wherein the waste container is fluidically connected to one or more sample containers, directly or indirectly.

20. The apparatus of any one of embodiments 1-19, wherein at least one of the sample container(s) comprises a porous means or a porous membrane to allow a liquid to pass through and evacuate the sample container and/or to maintain a sample, e.g., a sample liquid, in the sample container.

21. The apparatus of any one of embodiments 1-20, wherein at least one of the sample container(s) comprises a filter means or a filter positioned and configured to minimize or block escape of a sample, e.g., a sample liquid, from the sample container.

22. The apparatus of embodiment 20 or 21, wherein the porous means or filter means comprises a frit.

23. The apparatus of embodiment 22, wherein the frit has a pore size from about 1 μm to about 500 μm.

24. The apparatus of embodiment 22, wherein the frit has a pore size of about less than 50 μm.

25. The apparatus of any one of embodiments 21-24, wherein the filter means or filter comprises or is made of polytetrafluoroethylene (PTFE) or polyethylene (PE).

26. The apparatus of any one of embodiments 1-25, wherein at least one of the sample container(s) is open to atmospheric pressure.

27. The apparatus of any one of embodiments 1-26, wherein the supply line connecting the reagent reservoirs to the sample container(s) is a common line.

28. The apparatus of any one of embodiments 1-27, wherein at least one of the sample container(s) is configured to be loaded with a starting sample, e.g., a starting sample liquid.

29. The apparatus of any one of embodiments 1-28, wherein two or more of the valves are integrated in a manifold.

30. The apparatus of any one of embodiments 1-29, which further comprises a means for accelerating a reaction in at least one of the sample container(s).

31. The apparatus of embodiment 30, wherein the means for accelerating the reaction is configured to apply microwave energy to accelerate the reaction in at least one of the sample container(s).

32. The apparatus of any one of embodiments 1-31, which further comprises a processor means and a control program, said processor means being configured to operate the control program to control temperature of the sample container(s), temperature of the reagent reservoir(s), positioning of the valve(s), delivery of the one or more reagent(s) to the sample container(s), and/or evacuation of the content of the sample container(s).

33. The apparatus of any one of embodiments 1-32, which further comprises a display and an input means by a user.

34. The apparatus of any one of embodiments 1-33, which further comprises a means for monitoring the apparatus.

35. The apparatus of embodiment 34, wherein the monitoring means is configured to monitor temperature, pressure, flow, air bubble, position of one or more of the valves, refractive index, and/or conductance.

36. The apparatus of any one of embodiments 32-35, which is configured to provide feedback of the monitoring to the control program.

37. The apparatus of any one of embodiments 1-36, which further comprises an illumination means.

38. The apparatus of any one of embodiments 1-37, which further comprises a means or a sensor for detecting a detectable signal, e.g., a fluorescent signal.

39. The apparatus of any one of embodiments 1-38, which further comprises a detector for detecting a machine-readable signal, e.g., a barcode reader.

40. The apparatus of any one of embodiments 1-39, which further comprises a means for collecting the sample or a portion thereof.

41. The apparatus of embodiment 40, wherein the means for collecting the sample or a portion thereof comprises a collection container connected, directly or indirectly, to at least one of the sample container(s).

42. The apparatus of any one of embodiments 1-41, which comprises a single sample container that is subjected to temperature control and configured for allowing fluid flow-through, or a holder or space configured for holding the single sample container.

43. The apparatus of any one of embodiments 1-41, which comprises multiple sample containers, wherein at least one of the sample containers is subjected to temperature control and configured for allowing fluid flow-through, or a holder or space configured for holding the sample containers.

44. The apparatus of any one of embodiments 1-41, which comprises multiple sample containers that are subjected to temperature control and configured for allowing fluid flow-through, or a holder or space configured for holding the multiple sample containers.

45. The apparatus of any one of embodiments 1-44, wherein a single reagent reservoir is subjected to temperature control.

46. The apparatus of any one of embodiments 1-44, wherein multiple reagent reservoirs are subjected to temperature control.

47. The apparatus of any one of embodiments 1-46, wherein a single valve is positionable to provide alternate flow paths therethrough.

48. The apparatus of any one of embodiments 1-46, wherein multiple valves are positionable to provide alternate flow paths therethrough.

49. The apparatus of any one of embodiments 1-48, wherein the control unit controls delivery of a single reagent to a single sample container.

50. The apparatus of any one of embodiments 1-48, wherein the control unit controls delivery of a single reagent to multiple sample containers.

51. The apparatus of any one of embodiments 1-48, wherein the control unit controls delivery of multiple reagents to multiple sample containers.

52. The apparatus of any one of embodiments 1-51, wherein delivery of a single reagent is individually addressable.

53. The apparatus of any one of embodiments 1-51, wherein delivery of multiple reagents is individually addressable.

54. The apparatus of any one of embodiments 1-53, wherein one supply line connects a single reagent reservoir to a single sample container.

55. The apparatus of any one of embodiments 1-53, wherein one supply line connects a single reagent reservoir to multiple sample containers.

56. The apparatus of any one of embodiments 1-53, wherein one supply line connects multiple reagent reservoirs to a single sample container.

57. The apparatus of any one of embodiments 1-53, wherein one supply line connects multiple reagent reservoirs to multiple sample containers.

58. The apparatus of any one of embodiments 1-57, wherein at least two or three of temperature control of the sample container(s), temperature control of the reagent reservoir(s), positioning of the valve(s) and/or delivery of the one or more reagent(s) to the sample container(s) are automated and controlled by the control unit.

59. The apparatus of any one of embodiments 1-57, wherein temperature control of the sample container(s), temperature control of the reagent reservoir(s), positioning of the valve(s) and delivery of the one or more reagent(s) to the sample container(s) are automated and controlled by the control unit.

60. The apparatus of any one of embodiments 1-59, which comprises at least one reagent reservoir comprising a binding agent, or a holder or space configured for holding the reagent reservoir.

61. The apparatus of any one of embodiments 1-60, which comprises at least one reagent reservoir comprising reagents for transferring information, or a holder or space configured for holding the reagent reservoir.

62. The apparatus of any one of embodiments 1-61, which comprises at least one reagent reservoir comprising reagents for removing a terminal amino acid of a polypeptide, or a holder or space configured for holding the reagent reservoir.

63. The apparatus of any one of embodiments 1-62, which comprises at least one reagent reservoir comprising reagents for a capping reaction, or a holder or space configured for holding the reagent reservoir.

64. The apparatus of any one of embodiments 1-63, which comprises at least two reagent reservoirs, the reagent reservoirs comprising different types of reagents, and each of the reagent reservoirs comprising a reagent selected from the group consisting of a binding agent, reagents for transferring information, reagents for removing a terminal amino acid of a polypeptide and reagents for a capping reaction, or holders or spaces configured for holding the reagent reservoirs.

65. The apparatus of any one of embodiments 1-63, which comprises at least three reagent reservoirs, the reagent reservoirs comprising different types of reagents, and each of the reagent reservoirs comprising a reagent selected from the group consisting of a binding agent, reagents for transferring information, reagents for removing a terminal amino acid of a polypeptide and reagents for a capping reaction, or holders or spaces configured for holding the reagent reservoirs.

66. The apparatus of any one of embodiments 1-63, which comprises at least one reagent reservoir comprising a binding agent, at least one reagent reservoir comprising reagents for transferring information, at least one reagent reservoir comprising reagents for removing a terminal amino acid of a polypeptide, and at least one reservoir comprising reagents for a capping reaction, or holders or spaces configured for holding the reagent reservoirs.

67. The apparatus of any one of embodiments 60-66, wherein at least one of the reagent reservoirs comprising a binding agent, reagents for transferring information, reagents for removing a terminal amino acid of a polypeptide, and reagents for a capping reaction, or a holder or space configured for holding the reagent reservoir, is subjected to temperature control.

68. The apparatus of any one of embodiments 60-66, wherein at least two or three of the reagent reservoirs comprising a binding agent, reagents for transferring information, reagents for removing a terminal amino acid of a polypeptide, and reagents for a capping reaction, or holders or spaces configured for holding the reagent reservoirs, are subjected to temperature control.

69. The apparatus of any one of embodiments 60-66, wherein the reagent reservoir comprising a binding agent, the reagent reservoir comprising reagents for transferring information, the reservoir comprising reagents for removing a terminal amino acid of a polypeptide, and the reservoir comprising reagents for a capping reaction, or holders or spaces configured for holding the reagent reservoirs, are subjected to temperature control.

70. The apparatus of any one of embodiments 1-69, wherein at least one of the reagent reservoirs comprises a wash buffer.

71. The apparatus of embodiment 70, which comprises a single reagent reservoir that comprises a wash buffer.

72. The apparatus of embodiment 70, which comprises multiple reagent reservoirs that comprise different wash buffers, e.g., three or more different wash buffers.

73. The apparatus of any one of embodiments 70-72, wherein the reagent reservoir comprising the wash buffer is configured to hold a volume of about 50 mL or more.

74. The apparatus of any one of embodiments 1-73, wherein the sample container(s) is loaded with a sample containing a macromolecule, e.g., a polypeptide.

75. The apparatus of embodiment 74, wherein the macromolecule is a protein.

76. The apparatus of embodiment 74, wherein the macromolecule is a peptide.

77. The apparatus of embodiment 74, wherein the sample comprises a plurality of polypeptides, e.g., multiple proteins or peptides.

78. The apparatus of embodiment 76, wherein the peptide is obtained by fragmenting a protein, e.g., a protein from a biological sample.

79. The apparatus of any one of embodiments 74-78, wherein the macromolecule is associated with or joined to a recording tag.

80. The apparatus of embodiment 79, wherein the recording tag is a DNA molecule, an RNA molecule, a PNA molecule, a BNA molecule, an XNA molecule, an LNA molecule, a γPNA molecule, or a combination thereof.

81. The apparatus of embodiment 79 or 80, wherein the recording tag comprises a universal priming sequence.

82. The apparatus of any one of embodiments 79-81, wherein the macromolecule, the associated or joined recording tag, or both, are covalently joined to a solid support.

83. The apparatus of embodiment 82, wherein the solid support is a three-dimensional support (e.g., a porous matrix or a bead).

84. The apparatus of embodiment 82, wherein the solid support is a polystyrene bead, a polyacrylate bead, a polymer bead, an agarose bead, a cellulose bead, a dextran bead, an acrylamide bead, a solid core bead, a porous bead, a paramagnetic bead, a glass bead, a controlled pore bead, a silica-based bead, or a combination thereof.

85. The apparatus of any one of embodiments 60-84, wherein the binding agent is a polypeptide or protein.

86. The apparatus of embodiment 85, wherein the binding agent is a modified aminopeptidase, a modified amino acyl tRNA synthetase, a modified anticalin, or an antibody or a binding fragment thereof.

87. The apparatus of any one of embodiments 60-86, wherein the binding agent is configured to bind a target comprising a single amino acid residue, a dipeptide, a tripeptide or a post-translational modification of a polypeptide.

88. The apparatus of embodiment 87, wherein the binding agent is configured to bind a target comprising an N-terminal amino acid residue, a C-terminal amino acid residue, or an internal amino acid residue of a polypeptide.

89. The apparatus of embodiment 88, wherein the binding agent is configured to bind a target comprising a modified N-terminal amino acid residue, a modified C-terminal amino acid residue, or a modified internal amino acid residue of a polypeptide.

90. The apparatus of embodiment 87, wherein the binding agent is configured to bind a target comprising an N-terminal peptide, a C-terminal peptide, or an internal peptide of a polypeptide.

91. The apparatus of any one of embodiments 60-90, wherein the binding agent comprises a coding tag with identifying information regarding the binding agent.

92. The apparatus of embodiments 91, wherein the coding tag is DNA molecule, an RNA molecule, a BNA molecule, an XNA molecule, a LNA molecule, a PNA molecule, a γPNA molecule, or a combination thereof.

93. The apparatus of embodiment 91 or embodiment 92, wherein the coding tag comprises an encoder sequence.

94. The apparatus of any one of embodiments 91-93, wherein the coding tag further comprises a spacer, a binding cycle specific sequence, a unique molecular identifier, a universal priming site, or a combination thereof.

95. The apparatus of any one of embodiments 91-94, wherein the binding agent and the coding tag are joined by a linker.

96. The apparatus of any one of embodiments 79-95, which further comprises a reagent for amplifying the recording tag.

97. The apparatus of any one of embodiments 61-96, wherein the reagents for transferring information comprises an enzyme.

98. The apparatus of embodiment 97, wherein the reagent for transferring information is for performing a primer extension or ligation reaction.

99. The apparatus of embodiment 97 or 98, wherein the reagents for transferring information is subject to temperature control.

100. The apparatus of any one of embodiments 63-99, wherein the reagents for the capping reaction comprises a capping nucleic acid.

101. The apparatus of embodiment 100, wherein the capping nucleic acid comprises a universal priming sequence.

102. The apparatus of embodiment 100 or 101, wherein the reagents for the capping reaction comprises an enzyme.

103. The apparatus of 102, wherein the capping reagent is for performing an extension or ligation reaction.

104. The apparatus of any one of embodiments 100-103, wherein the reagents for the capping reaction is subject to temperature control.

105. The apparatus of any one of embodiments 62-104, wherein the reagents for removing a terminal amino acid of a polypeptide comprises a chemical or enzymatic reagent.

106. The apparatus of any one of embodiments 1-105, which further comprises:
a) a reagent for modifying a terminal amino acid of a polypeptide; or
b) a reagent reservoir comprising a reagent for modifying a terminal amino acid of a polypeptide.

107. The apparatus of embodiment 106, wherein the reagent for modifying a terminal amino acid of a polypeptide comprises a chemical agent or an enzymatic agent.

108. The apparatus of any one of embodiments 1-107, wherein at least one of the valves has a dead volume from about 0.5 μL to about 5 μL, e.g., from about 1 μL to about 2 μL.

109. The apparatus of any one of embodiments 1-108, wherein the control unit is configured to be operated using a cross-platform language, e.g., python.

110. The apparatus of any one of embodiments 1-109, which is configured to be operated without real-time control or without precise real-time control.

111. The apparatus of any one of embodiments 1-110, wherein at least one of the reagent reservoirs with a smaller volume is located closer to the sample container(s) than a reagent reservoir with a larger volume.

112. The apparatus of embodiment 111, wherein at least one of the reagent reservoirs comprising a binding agent, reagents for transferring information, reagents for removing a terminal amino acid of a polypeptide and/or reagents for a capping reaction is located closer to the sample container(s) than a reagent reservoir comprising a wash buffer.

113. The apparatus of any one of embodiments 1-112, which is configured to integrate an aqueous-phase biochemical reaction and an organic chemical reaction into a cyclic process, e.g., a cyclic process for converting a peptide sequence into a DNA library for NGS analysis.

114. The apparatus of any one of embodiments 1-113, which is configured to generate an output sample, e.g., an output sample comprising a DNA library or an encoded library, that is configured to be analyzed by a DNA sequencer, e.g., a general purpose DNA sequencer (NGS).

115. The apparatus of any one of embodiments 1-114, which is configured to perform high-throughput sample processing.

116. The apparatus of any one of embodiments 1-115, which is configured to perform polypeptide-agnostic or protein-agnostic analysis.

117. A method for automated treatment of a sample, which method is conducted using an apparatus of any one of embodiments 1-116, and which method comprises:
a) providing a non-planar sample container comprising a sample comprising a macromolecule, e.g., a polypeptide, and an associated recording tag joined to a solid support to said apparatus;
b) providing a binding agent and reagents for transferring information to separate reagent reservoirs of said apparatus, wherein at least one of said reagent reservoirs comprises a binding agent and at least one of said reagent reservoirs comprises reagents for transferring information;
c) delivering the binding agent from the reagent reservoir to the sample container, wherein the binding agent comprises a coding tag with identifying information regarding the binding agent; and
d) delivering the reagents for transferring information from the reagent reservoir to the sample container to transfer information from the coding tag of the binding agent to the recording tag to generate an extended recording tag.

118. The method of embodiment 117, which further comprises repeating steps c) and d) two or more times.

119. The method of embodiment 117 or embodiment 118, wherein the sample container(s) is provided with a sample with a volume equal to or less than about 20 mL.

120. The method of embodiment 117 or embodiment 118, wherein the sample container(s) is provided with a sample with a volume equal to or less than about 10 mL.

121. The method of any one of embodiments 117-120, wherein the recording tag is a DNA molecule, an RNA molecule, a PNA molecule, a BNA molecule, an XNA molecule, an LNA molecule, a γPNA molecule, or a combination thereof.

122. The method of embodiment 121, wherein the recording tag comprises a universal priming sequence.

123. The method of any one of embodiments 117-122, wherein the macromolecule, the associated or joined recording tag, or both, are covalently joined to a solid support.

124. The method of embodiment 123, wherein the solid support is a three-dimensional support (e.g., a porous matrix or a bead).

125. The method of embodiment 124, wherein the solid support is a polystyrene bead, a polyacrylate bead, a polymer bead, an agarose bead, a cellulose bead, a dextran bead, an acrylamide bead, a solid core bead, a porous bead, a paramagnetic bead, a glass bead, a controlled pore bead, a silica-based bead, or a combination thereof.

126. The method of any one of embodiments 117-125, wherein transferring the information of the coding tag to the recording tag is mediated by a DNA ligase.

127. The method of any one of embodiments 117-126, wherein transferring the information of the coding tag to the recording tag is mediated by a DNA polymerase.

128. The method of any one of embodiments 117-126, wherein transferring the information of the coding tag to the recording tag is mediated by a chemical ligation.

129. The method of any one of embodiments 117-128, further comprising providing reagents for removing a terminal amino acid of a polypeptide to a separate reagent reservoir of said apparatus in step a) and step:
e) delivering the reagents for removing a terminal amino acid of a polypeptide from the reagent reservoir to the sample container to remove the terminal amino acid.

130. The method of embodiment 129, wherein step e) is performed after step a) and step b).

131. The method of embodiment 129 or embodiment 130, which further comprises repeating steps c) to e) two or more times.

132. The method of any one of embodiments 117-131, further comprising providing reagents for a capping reaction to a separate reagent reservoir of said apparatus in step a) and step:
f) delivering the reagents for a capping reaction from the reagent reservoir to the sample container.

133. The method of embodiment 132, wherein step f) is performed after steps a) to e).

134. The method of embodiment 132 or embodiment 133, wherein the reagents for a capping reaction comprises a universal priming sequence and reagents for an extension or ligation reaction.

135. The method of any one of embodiments 117-134, further comprising providing a reagent for modifying a terminal amino acid of a polypeptide to the reagent reservoir of said apparatus in step a) and delivering the reagent for modifying a terminal amino acid of a polypeptide to the sample container.

136. The method of embodiment 135, wherein the reagent for modifying a terminal amino acid of a polypeptide comprises a chemical agent or an enzymatic agent.

137. The method of embodiment 135 or embodiment 136, wherein the reagent for modifying a terminal amino acid of a polypeptide is delivered to the sample container before step c), before step d), before step e), and/or before step f).

138. The method of any one of embodiments 117-137, which further comprises releasing and collecting the sample from the sample container or a portion thereof.

139. The method of any one of embodiments 117-138, which further comprises amplifying the extended recording tag.

140. The method of any one of embodiments 117-139, wherein performing any of steps c)-f) comprises adjusting the temperature of the sample container.

141. The method of any one of embodiments 129-140, wherein performing step e) comprises adjusting the temperature of the sample container to a temperature between about 25° C. to about 60° C.

142. The method of any one of embodiments 117-141, which further comprises delivering a wash buffer from the reagent reservoir to the sample container.

143. The method of embodiment 142, wherein the wash buffer is delivered before step c), before step d), before step e), and/or before step f).

144. The method of embodiment 142 or embodiment 143, which comprises delivering a single wash buffer from the reagent reservoir to the sample container.

145. The method of embodiment 142 or embodiment 143, which comprises delivering multiple wash buffers, e.g., from 2 to 10 wash buffers from the reagent reservoirs to the sample container.

146. The method of any one of embodiments 117-145, wherein at least one of the steps c)-f) is conducted with one or more controlled flow rates.

147. The method of any one of embodiments 117-146, wherein at least one of the steps c)-f) is controlled by the control unit.

148. The method of embodiment 147, wherein two, three or all of the steps c)-f) are controlled by the control unit.

149. The method of any one of embodiments 117-148, wherein at least one of the steps a)-f) is automated.

150. The method of embodiment 149, wherein two, three, four, five or all of the steps a)-f) are automated.

151. The method of any one of embodiments 117-150, further comprising collecting the sample or a portion thereof in a collection container connected, directly or indirectly, to at least one of the sample container(s).

152. The method of embodiment 151, wherein the sample is treated with a cleaving reagent prior to collecting the sample or a portion thereof in the collection container.

153. The method of embodiment 151 or embodiment 152, wherein the collecting is automated and/or controlled by the control unit.

154. The method of any one of embodiments 117-153, wherein the control unit is operated using a cross-platform language, e.g., python.

155. The method of any one of embodiments 117-154, which is operated without real-time control or without precise real-time control.

156. The method of any one of embodiments 117-155, which integrates an aqueous-phase biochemical reaction and an organic chemical reaction into a cyclic process, e.g., a cyclic process for converting a peptide sequence into a DNA library for NGS analysis.

157. The method of any one of embodiments 117-156, which generates an output sample, e.g., an output sample comprising a DNA library or an encoded library, that is analyzed by a DNA sequencer, e.g., a general purpose DNA sequencer (NGS).

158. The method of any one of embodiments 117-157, which is conducted to perform high-throughput sample processing.

159. The method of any one of embodiments 117-158, which is conducted to perform polypeptide-agnostic or protein-agnostic analysis.

### VI. EXAMPLES

The following examples are offered to illustrate but not to limit the methods, compositions, and uses provided herein.

### Example 1: Integrated ProteoCode Assay on Exemplary Automated Apparatus

This experiment describes treatment of polypeptides performed using an exemplary instrument for a ProteoCode assay that includes multicycle encoding. The experiment included the following steps: binding/encoding→chemistry→binding/encoding→chemistry→binding/encoding→end-capping. Programmed automated processes for binding, encoding, cleaving using chemistry treatment and performing the endcap reaction were carried out by a control unit connected to the instrument, with the binding/encoding and cleaving processes repeated using a controlled loop. Among other features described in the processes below, the instrument used for this experiment has two 7-way rotary valves and a microvalve with four ports. The instrument used can be loaded with up to 14 reagents and 2 sample cartridges that are subjected to active heating and cooling.

Sample Loading and Pre-Washing

Two cartridges were inserted into a temperature controlled thermal-block on the instrument. To each cartridge, 100 μL of peptides labelled with a DNA recording tag immobilized on a substrate was added. Each sample loaded into the cartridge contained 50,000 beads, and peptides labelled with a DNA recording tag were loaded on porous beads at a controlled density of one activated functional moiety for attaching the peptide-recording tag chimera per 100,000 passivated (blocked) molecules (1:100K). Each cartridge contained a PTFE frit (5.1 mm diameter, 3 mm thickness, and 3 μm pore size) such that the sample containing polypeptides immobilized on beads is retained in the cartridge and liquids, wash solutions, and reagents delivered to the cartridge can be removed by positive pressure applied to the cartridge. A pump and valve(s) integrated on the instrument were used to control dispensing and flow of the reagents on the system and delivery of reagents to the sample in the cartridges. Flow-through removed from the cartridges were dispensed into a waste container.

Exemplary peptides tested in the assay included peptides with an N-terminal amino FS (FS-peptide, FSGVAMPGAEDDVVGSGSK set forth in SEQ ID NO: 3); peptides with an N-terminal amino AFS (AFS-peptide, AFSGVAMPGAEDDVVGSGSK set forth in SEQ ID NO: 4), and peptides with an N-terminal amino AEFS (AEFS-peptide, AEFSGVAMPGAEDDVVGSGSK set forth in SEQ ID NO: 5). Prior to initiating the first binding and encoding process, the beads were pre-washed in the cartridge with 200 μL of PBF10 (10% formamide, 4 mM sodium phosphate, 500 mM sodium chloride, and 0.1% Tween 20), followed by 4 washes of 200 μL of PBST (4 mM sodium phosphate, 155 mM sodium chloride (NaCl), and 0.1% Tween 20) to remove non-specifically bound peptides and DNA not immobilized on beads.

One Cycle of Binding and Encoding

Each cycle of binding/encoding is performed as follows using the instrument and exemplary programmed automated binding and encoding processes. The thermal-block was set to 25° C. (+/−1° C.). Once the set temperature is reached, 200 μL of an exemplary binding agent that binds phenylalanine when it is the N-terminal amino acid residue (F-binder) were delivered to the beads in the cartridge and incubated for 30 minutes. The binding agents were conjugated with a coding tag oligo containing information regarding the binding agent. After the binding agent bound its corresponding target, an N-terminal F amino acid, the 3′-spacer′ region of the coding tag hybridized to the 3′-spacer of the recording tag oligo linked with the peptide. After 30 minutes of incubation, the beads were washed 4 times with 200 μL of Binder Wash Buffer (BWH, 4 mM sodium phosphate, 500 mM sodium chloride, 0.1% Tween 20) and 1 time with 200 μL of Custom Encoding Buffer (CB, 50 mM Tris-HCl pH 7.5, 2 mM MgSO4, 50 mM NaCl, 1 mM DTT, 0.1% Tween 20, 100 μg/mL BSA). For transfer of information from the coding tag to the recording tag, a total of 400 μL (2×200 μL) of Encoding Master Mix (EMM, 50 mM Tris-HCl pH 7.5, 2 mM MgSO4, 50 mM NaCl, 1 mM DTT, 0.1% Tween 20, 100 μg/mL BSA, 0.125 mM dNTPs, 0.125 U/μL Klenow fragment (3′—>5′ exo-) (MCLAB, USA)) was delivered to the beads and incubated for 5 minutes at 25° C. If the binding agent bound its target, the recording tag associated with the polypeptide was elongated by copying the coding tag by extension and information was transferred from the coding tag associated with the F binding agent to the recording tag linked to the peptide (thereby forming an extended recording tag). After the 5 minute incubation, the beads were washed 5 times with 200 μL of PBF10, 5 times with 200 μL of 0.1 M sodium hydroxide with 0.1% Tween 20, 5 times with 200 μL of PBF10 and 5 times with 200 μL of PBST.

One Cycle of Chemistry Treatment

Each cycle of chemistry is performed as follows using the instrument and exemplary automated programmed process. Following one cycle of binding and encoding as described above, the thermal-block was set to 40° C. (+/−1° C.). While the thermal-block is being ramped-up to the set temperature, the beads were pre-washed with 4×200 μL of a reagent for functionalization of the N-terminal amino acid. Once the thermal-block has reached 40° C., 200 μL of reagent for functionalization were delivered to the beads and incubated for 30 minutes to functionalize the N-terminal amino acid (NTAA) on the beads. The beads were washed multiple times then pre-washed with 4×200 μL of reagent for eliminating or cleaving the NTAA and incubated with the same reagent to remove the functionalized NTAA. The temperature was set to 30° C. (+/−10° C.) and the beads were washed 5 times with 1 mL of PBST after the 60 minute incubation.

As a control, samples that were not treated with the reagent for cleaving the NTAA was treated with a PBST solution in place of the reagent for functionalization and reagent for eliminating the NTAA.

End-Capping

The following describes the end-capping process performed on the instrument using an exemplary automated programmed process for end-capping. Once the final round of encoding (third encoding cycle) was completed, 200 μL of an End-Capping solution (CAP, 400 nM capping oligo, 50 mM Tris-HCl pH 7.5, 2 mM MgSO4, 50 mM NaCl, 1 mM DTT, 0.1% Tween 20, 100 μg/mL BSA, 0.125 mM dNTPs, 0.125 U/μL Klenow exo-) were delivered to the beads. The capping oligo provided in this step contained a universal priming sequence which is added to the recording tag using an extension reaction to generate a final product for NGS readout. The beads were incubated in the end-capping solution for 10 minutes at 25° C. and washed 5 times with 200 μL of PBF10, 5 times with 200 μL of 0.1 M sodium hydroxide with 0.1% Tween 20, 5 times with 200 μL of PBF10 and 5 times with 200 μL of PBST.

Following the end-capping reaction, the cartridges were removed from the instrument and each sample (e.g., polypeptides immobilized on beads with the extended recording tags) was removed from the cartridge.

Sample Processing and Analysis

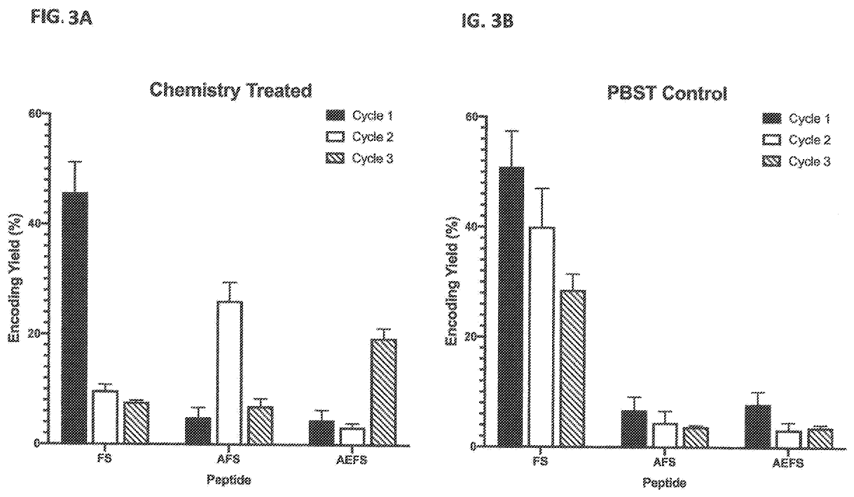

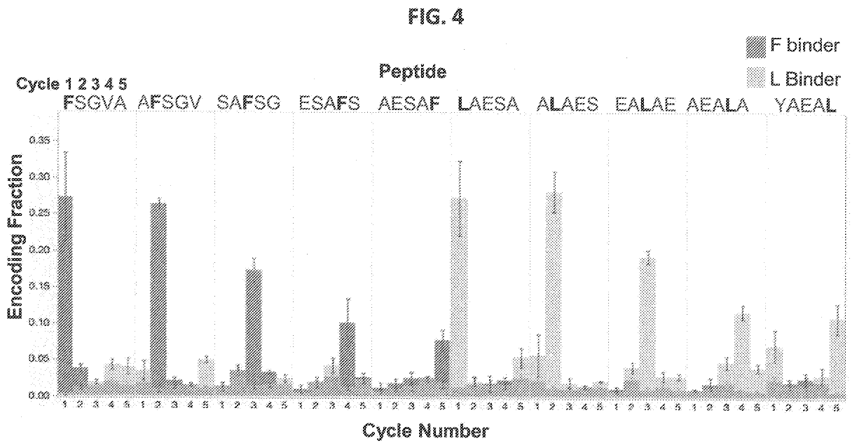

The extended recording tag of the assay was subjected to PCR amplification and analyzed by next-generation sequencing (NGS). The NGS results indicate that the chemistry treated sample (FIG. 3A) showed cycle-specific encoding of the F-peptide at cycle 1 (solid bar), cycle 2 (empty bar), and cycle 3 (lined bar). In the chemistry treated sample shown in FIG. 3A, the F-binder detected the N-terminal phenylalanine (F) in the FS-peptide in the 1st cycle, the N-terminal phenylalanine (F) in the AFS-peptide in the 2nd cycle once the original N-terminal alanine (A) was removed by the chemistry treatment, and the N-terminal F amino acid in the AEFS-peptide in the 3rd cycle once the alanine (A) and glutamic acid (E) amino acid was removed individually by each of the two rounds of chemistry treatment. In contrast, the control samples that were not exposed to chemistry treatment for functionalizing or removing the NTAA (FIG. 3B) showed no significant encoding on either the 2nd or 3rd position F amino acid of the tested peptides. In summary, the treatment of the polypeptides using the exemplary instrument resulted in successful treatment and processing of polypeptides and formation of extended recording tags containing polypeptide information that can be used to assess the amino acid sequence of the treated polypeptides.

### Example 2: Five Cycle ProteoCode Assay Using PMI Chemistry and aa Pool of F and L Binders on an Automated Apparatus

This example demonstrates a ProteoCode assay conducted on an Automated Apparatus including modification (e.g., functionalization) and elimination of the N-terminal amino acid (NTAA) of peptides treated with diheterocyclic methanimine (PMI) (See e.g., PCT/US2020/029969). Binding of a binding agent to the modified NTAA and encoding by transferring information from a coding tag associated with the binding agent to a recording tag associated with the peptide, thereby generating an extended recording tag, was also performed as shown in FIG. 4. Binding and encoding was performed using a pool of binding agents (phenylalanine (F) and leucine (L) binders) that recognize the modified NTAA (“mod”).

Five cycles of ProteoCode chemistry were performed on ProteoCode beads immobilized with 18 different peptides (SEQ ID NOs: 6-23). Beads were sampled after each cycle and resultant encoded libraries analyzed with NGS sequencing. In FIG. 4, summary NGS encoding data are shown for each of the 10 relevant F and L peptides for each cycle (only the first 5 residues shown). Plot of summary cycle-dependent encoding efficiency with mod-F-binder and mod-L binder detection. The F and L peptide sets are comprised of peptides with “laddered” F and L residues in positions 1-5. As each successive residue is removed in subsequent Edman-Lite cycles, a new NTAA is exposed. For example, a peptide with an F at the 5th position is decoded on the fifth cycle by F-binder encoding.

Peptides labelled with a DNA recording tag were immobilized on a substrate (peptide sequences as set forth in SEQ ID NOs: 6-23). Up to four cycles of elimination followed by binding and encoding were performed. For example, the peptides were treated with an exemplary diheterocyclic methanimine as the reagent for functionalization of the NTAA. For functionalization treatment, the assay beads were incubated with 150 μL of 15 mM of di-(4-trifluoromethyl-pyrazo-1-yl)methanimine, 200 mM MOPS, pH7.6, 50% DMA at 40° C. for 30 minutes. The beads were washed 3× with 200 μL of PBST. Following functionalization, the assay beads were subjected to treatment with 150 μL of 7% hydrazine hydrochloride in PBS, pH 7.0 at 40° C. for 30 min. After 3× PBST washes, the elimination treatment was performed by incubating the assay beads with 150 μL of 1 M ammonium phosphate, pH 6.0 at 95° C. for 30 min. The beads were then washed 3× with 200 μL of PBST. The first cycle of binding F and L-binder to the functionalized NTAA (4-trifluoromethylpyrazol-1-yl carboamidinyl)-peptide) and encoding was performed before any hydrazine treatment and elimination treatment (FIG. 4). A set of 18 different peptides labelled with a DNA recording tag were immobilized on a substrate (peptide sequences as set forth in SEQ ID NOs: 6-23). Up to five cycles of ProteoCode assay were performed comprised of functionalization, binding and encoding, and elimination. F and L-binder binding/encoding for subsequent cycles as indicated was performed after functionalization after either zero, one, two, three, or four cycles of elimination.

For example, the peptides were treated with an exemplary diheterocyclic methanimine as the reagent for functionalization of the NTAA. For functionalization treatment, the assay beads were incubated with 150 μL of 15 mM of di-(4-trifluoromethyl-pyrazo-1-yl)methanimine, 200 mM MOPS, pH7.6, 50% DMA at 40° C. for 30 minutes. The beads were washed 3× with 200 μL of PBST. Following functionalization, the assay beads were subjected to treatment with 150 μL of 7% hydrazine hydrochloride in PBS, pH 7.0 at 40° C. for 30 min. After 3× PBST washes, the elimination treatment was performed by incubating the assay beads with 150 μL of 1 M ammonium phosphate, pH 6.0 at 95° C. for 30 min. The beads were then washed 3× with 200 μL of PBST. The first cycle of binding F and L-binder to the functionalized NTAA (4-trifluoromethylpyrazol-1-yl carboamidinyl)-peptide) and encoding was performed before any hydrazine treatment and elimination treatment (FIG. 4).

The extended recording tag of the assay was subjected to PCR amplification and analyzed by next-generation sequencing (NGS). FIG. 4 shows chemistry cycle-dependent encoding efficiency with the mod-F-binder and mod-L binder detection for peptides with the 5 residues of the N-terminal end indicated. Data on ten F and L containing peptides, in which either the F or L residue is stepped through the first 5 positions of the peptide, is shown. As each successive residue was eliminated, an N-terminal modified F or L residue was exposed on one of the peptides on the bead and detected by the corresponding mod-F or mod-L binder with concomitant DNA encoding. As shown, functionalization and binding of the modified NTAA was observed as indicated by elevated encoding levels. It was also observed that elimination was achieved as each binder detected the corresponding modified residue in the appropriate cycle after elimination of other residues that exposed the F or L residue. In summary, an increase in F-binder and L-binder encoding after functionalization (NTF) was observed and elimination (NTE) was detected, demonstrating the use of the exemplary diheterocyclic methanimine in the encoding assay for elimination of the NTAA and as a modification recognized by the shown exemplary binding agents.

TABLE 1
Assay Peptides

| SEQ | |
|---|---|
| ID NO | Sequence |
| 6 | YAEALAESAFSGVARGDVRGGK(N3) |
| 7 | AEALAESAFSGVARGDVRGGK(N3) |
| 8 | EALAESAFSGVARGDVRGGK(N3) |
| 9 | ALAESAFSGVARGDVRGGK(N3) |
| 10 | LAESAFSGVARGDVRGGK(N3) |
| 11 | AESAFSGVARGDVRGGK(N3) |
| 12 | ESAFSGVARGDVRGGK(N3) |
| 13 | SAFSGVARGDVRGGK(N3) |
| 14 | AFSGVARGDVRGGK(N3) |
| 15 | FSGVARGDVRGGK(N3) |
| 16 | SGVARGDVRGGK(N3) |
| 17 | LAGELAGELAGEIRGDVRGGK(N3) |
| 18 | ELAGELAGELAGEIRGDVRGGK(N3) |
| 19 | GELAGELAGELAGEIRGDVRGGK(N3) |
| 20 | AGELAGELAGELAGEIRGDVRGGK(N3) |
| 21 | FAFAGVAMPRGAEDVRGGK(N3) |
| 22 | FLAEIRGDVRGGK(N3) |
| 23 | dimethyl-AESAESASRFSGVAMPGAEDDVVGSGSK(N3) |

The present disclosure is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure. These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

SEQUENCE TABLE

| SEQ ID | | |
|---|---|---|
| NO | Sequence (5′-3′) | Description |
| 1 | AATGATACGGCGACCACCGA | P5 primer |
| 2 | CAAGCAGAAGACGGCATACGAGAT | P7 primer |
| 3 | FSGVAMPGAEDDVVGSGSK | FS-peptide |
| 4 | AFSGVAMPGAEDDVVGSGSK | AFS-peptide |
| 5 | AEFSGVAMPGAEDDVVGSGSK | AEFS-peptide |

1. An apparatus for automated treatment of a sample comprising an immobilized macromolecule analyte, which apparatus comprises:
one or more non-planar sample container(s) with a volume equal to or less than about 20 mL, wherein the one or more non-planar sample container(s) is/are characterized by having a ratio between a height and a largest dimension from about 1:1 to about 100:1: and at least one of said sample container(s) is subjected to temperature control and configured for allowing fluid flow-through, or a holder or space configured for holding said sample container(s);
a plurality of reagent reservoirs for containing a respective reagent, wherein at least one of said reagent reservoirs is subjected to temperature control, and contains an enzyme as a reagent;
a plurality of valves connected in a supply line having an upstream end and a downstream end, wherein at least one or each of said valves is positionable to provide alternate flow paths therethrough; and
a control unit to control delivery of said one or more reagent(s) to said sample container(s),
wherein:
said apparatus is configured to hold at least 5 reagent reservoirs:
delivery of said one or more reagent is individually addressable,
said supply line connects said reagent reservoirs to said sample container(s) and said reagent reservoirs are fluidically connected to said sample container(s), and
at least temperature control of said sample container(s), temperature control of said reagent reservoir(s), positioning of said valve(s) and/or delivery of said one or more reagent(s) to said sample container(s) is automated and controlled by said control unit.

2. The apparatus of claim 1, wherein at least one of the sample container(s) is subjected to active heating and active cooling; and at least one of the reagent reservoirs is subjected to active heating and/or active cooling.

3. The apparatus of claim 2, wherein the temperature of the sample container(s) subjected to temperature control and the temperature of the reagent reservoir(s) subjected to temperature control are individually controlled by the control unit, and the sample container(s) subjected to temperature control and the reagent reservoir(s) subjected to temperature control are housed in separate thermal blocks.

4. The apparatus of claim 1, which further comprises at least one pump for delivering the one or more reagents to the sample container(s).

5-7. (canceled)

8. The apparatus of claim 1, wherein the apparatus is configured to hold at least 10 reagent reservoirs.

9. The apparatus of claim 1, wherein the apparatus is configured to hold at least 20 reagent reservoirs.

10. (canceled)

11. The apparatus of claim 1, wherein the apparatus is configured to hold two or more sample containers, each is subjected to temperature control and configured for allowing fluid flow-through.

12. (canceled)

13. The apparatus of claim 1, wherein at least one of the sample container(s) comprises:
a porous means, a porous membrane or a frit to allow a liquid to pass through and evacuate the sample container, while maintaining the immobilized macromolecule in the sample container.

14. (canceled)

15. The apparatus of claim 1, which further comprises a means for accelerating a reaction in at least one of the sample container(s), wherein the means for accelerating the reaction is configured to apply microwave energy to accelerate the reaction in the at least one of the sample container(s).

16. (canceled)

17. The apparatus of claim 1, which further comprises a means for monitoring the apparatus, wherein the monitoring means is configured to monitor temperature, pressure, flow, air bubble formation, position of one or more of the valves, refractive index and conductance.

18. The apparatus of claim 1, which further comprises a sensor for detecting a fluorescent signal.

19-23. (canceled)

24. The apparatus of claim 1, which comprises at least one reagent reservoir comprising a binding agent, at least one reagent reservoir comprising a reagent for transferring information, at least one reagent reservoir comprising a reagent for removing a terminal amino acid of a polypeptide, and at least one reservoir comprising a reagent for a capping reaction.

25. The apparatus of claim 24, wherein at least two of the reagent reservoirs comprising a binding agent, reagents for transferring information, reagents for removing a terminal amino acid of a polypeptide, and reagents for a capping reaction are subjected to temperature control.

26. The apparatus of claim 1, which is for treating a plurality of polypeptides, wherein the sample container(s) is loaded with a sample comprising the plurality of polypeptides, and each polypeptide of the plurality of polypeptides is associated with a nucleic acid recording tag.

27. The apparatus of claim 26, wherein each polypeptide of the plurality of polypeptides is covalently joined to a solid support.

28. The apparatus of claim 26, wherein the apparatus comprises at least one reagent reservoir comprising a binding agent; the binding agent comprises a protein or an aptamer; and the binding agent is configured to bind a target comprising a single terminal amino acid residue, a dipeptide, a tripeptide or a post-translational amino acid modification of a polypeptide from the plurality of polypeptides.

29. The apparatus of claim 28, wherein the binding agent further comprises a coding tag with identifying information regarding the binding agent, wherein the coding tag is DNA molecule, an RNA molecule, a BNA molecule, an XNA molecule, a LNA molecule, a PNA molecule, a γPNA molecule, or a combination thereof.

30. The apparatus of claim 1, wherein at least one of the reagent reservoirs with a smaller volume is located closer to the sample container(s) than a reagent reservoir with a larger volume.

31. The apparatus of claim 1, which is configured to generate an output sample comprising a nucleic acid encoded library with information that represents a binding history of the macromolecule analyte, wherein the nucleic acid encoded library is compatible for analysis with a DNA sequencer.

32. The apparatus of claim 31, which is configured to perform high-throughput sample processing.

33. A method for automated treatment of a sample, which method is conducted using an apparatus of claim 1, and which method comprises:
a) providing to said apparatus a sample in a non-planar sample container, wherein the sample comprises a macromolecule analyte and an associated nucleic acid recording tag joined to a solid support;
b) providing a binding agent and a reagent for transferring information to separate reagent reservoirs of said apparatus, wherein at least one of said reagent reservoirs comprises the binding agent and at least one of said reagent reservoirs comprises the reagent for transferring information;
c) delivering the binding agent from the reagent reservoir to the sample container, wherein the binding agent comprises a coding tag with identifying information regarding the binding agent;
d) delivering the reagent for transferring information from the reagent reservoir to the sample container to transfer information from the coding tag of the binding agent to the recording tag to generate an extended recording tag; and
e) generating an output sample comprising a nucleic acid encoded library with information that represents a binding history of the macromolecule analyte, wherein the encoded library is compatible for analysis with a DNA sequencer.

34-159. (canceled)
